(19) United States
(12) Patent Application Publication
(10) Pub. No.: US 2025/0195474 A1
(43) Pub. Date: Jun. 19, 2025

(54) Piezo Inhibition for Wound Healing

(71) Applicant: The Board of Trustees of the Leland Stanford Junior University, Stanford, CA (US)

(72) Inventors: Michelle F. Griffin, Stanford, CA (US); Michael T. Longaker, Stanford, CA (US); Nicholas J. Guardino, Stanford, CA (US); Derrick C. Wan, Stanford, CA (US); Heather E. Talbott, Stanford, CA (US); Michael Januszyk, Stanford, CA (US)

(21) Appl. No.: 18/856,249

(22) PCT Filed: Apr. 18, 2023
(86) PCT No.: PCT/US2023/018997

§ 371 Date: Oct. 11, 2024

Related U.S. Application Data
(60) Provisional application No. 63/335,843, filed on Apr. 28, 2022.
(60) Provisional application No. 63/443,790, filed on Feb. 7, 2023.

(51) Int. Cl. A61K 31/409; A61K 9/00; A61K 45/06; A61P 17/02

(52) U.S. Cl. CPC A61K 31/409; A61K 9/0019; A61K 45/06; A61P 17/02

(57) ABSTRACT

Methods of promoting healing of a wound in a dermal location of a subject are provided. Aspects of the methods may include administering an effective amount of a Piezo inhibitor composition to the wound to promote healing of the wound, e.g., by reducing transition of adipocytes to fibroblasts in the wound. Also provided are methods of preventing or reversing scarring during healing of a wound in a subject. Aspects of the methods may include forming a wound in a dermal location of a subject and administering an effective amount of a Piezo inhibitor composition to the wound to promote regenerative healing or regenerative remodeling of the wound. Also provided are methods of ameliorating, e.g., reducing or inhibiting, organ fibrosis, e.g., liver fibrosis, heart fibrosis, inflammatory bowel fibrosis, muscle fibrosis, kidney fibrosis, etc., in a subject by administering to the subject an effective amount of a Piezo inhibitor composition. Also provided are kits including an amount of a Piezo inhibitor composition.

### CROSS-REFERENCE TO RELATED APPLICATIONS

Pursuant to 35 U.S.C. § 119 (e), this application claims priority to the filing dates of U.S. Provisional Application Ser. No. 63/335,843 filed on Apr. 28, 2022 and U.S. Provisional Application Ser. No. 63/443,790 filed on Feb. 7, 2023, the disclosures of which applications are herein incorporated by reference.

### ACKNOWLEDGEMENT OF GOVERNMENT RIGHTS

This invention was made with Government support under contract GM136659 awarded by the Advanced Research Projects Agency. The Government has certain rights in the invention.

### INTRODUCTION

The skin is the largest organ in the body consisting of several layers and plays an important role in biologic homeostasis. The skin has multiple functions, including thermal regulation, metabolic function (vitamin D metabolism), and immune functions. Mammalian skin includes two main layers, the epidermis and the dermis. The epidermis is outermost layer of skin and serves as a protective barrier to the environment. The dermis is the layer of skin beneath the epidermis and serves a location for the appendages of skin including, e.g., hair follicles, sweat glands, sebaceous glands, apocrine glands, lymphatic vessels and blood vessels. The dermis provides strength and elasticity to the skin through an extracellular matrix or connective tissue made of structural proteins (collagen and elastin), specialized proteins (fibrillin, fibronectin, and laminin), and proteoglycans. The epidermis and dermis are separated by the basement membrane, a thin, fibrous extracellular matrix.

Wound healing or tissue healing is a biological process that involves the replacement of damaged or destroyed tissue with living tissue. When the skin barrier is broken, a regulated sequence of biochemical events is activated to repair the damage. The process is regulated by numerous biological components including, e.g., growth factors, cytokines, and chemokines, and employs several components including, e.g., soluble mediators, blood cells, extracellular matrix components, and parenchymal cells. Wound healing generally proceeds through several stages. The process is divided into several phases including hemostasis, inflammation, proliferation, and remodeling. The end point of wound healing may include the formation of a scar. Skin wounds invariably heal by developing fibrotic scar tissue, which can result in disfigurement, growth restriction, and permanent functional loss. Various types of scars may form after skin tissue repair including, e.g., a “normal” fine line and abnormal scars including widespread scars, atrophic scars, scar contractures, hypertrophic scars, and keloid scars.

### SUMMARY

No current therapeutic strategies exist for successfully preventing or reversing the fibrotic process that leads to scarring. Attempts at reducing scarring often entail ablation of cell populations known to be fibrogenic, but this approach could impair or delay wound repair by nonspecifically eliminating cells that are needed for proper healing. Skin regeneration—as defined by recovery of three features of normal skin: 1) secondary elements (e.g., dermal appendages), 2) ECM structure, and 3) mechanical strength—has not been achieved.

Methods of promoting regenerative healing of a wound in a dermal location of a subject are provided. Aspects of the methods may include administering an effective amount of a Piezo inhibitor composition to the wound to promote regenerative healing of the wound, e.g., by reducing transition of adipocytes to fibroblasts in the wound. Also provided are methods of both preventing new scarring during healing of a wound in a subject, and reversing/resolving existing scarring of a healed wound in a subject. Aspects of the methods may include forming a wound in a dermal location of a subject and administering an effective amount of a Piezo inhibitor composition to the wound (to promote regenerative healing of the wound) or to a healed wound (i.e., scar; to promote regenerative remodeling of the wound and resolution/reversal of the scar). Also provided are methods of ameliorating, e.g., reducing or inhibiting, organ fibrosis, e.g., liver fibrosis, heart fibrosis, inflammatory bowel fibrosis and muscle fibrosis in a subject by administering to the subject an effective amount of a Piezo inhibitor composition. Also provided are kits including an amount of a Piezo inhibitor composition.

Also provided are method of ameliorating, e.g., reducing or inhibiting, organ fibrosis, e.g., liver fibrosis, heart fibrosis, inflammatory bowel fibrosis and muscle fibrosis.

### BRIEF DESCRIPTION OF THE FIGURES

FIG. 1: Adipocytes transition to fibroblasts in wounds and contribute to scarring. (A) Schematic of Adipoq^{Cre-ERT}; R26^{mTmG} wounding experiments for adipocyte lineage tracing. (B) Fluorescent histology of unwounded skin (UW) or wounds at indicated postoperative day (POD). White dotted regions highlight GFP⁺ cells within wounds. (C) Quantification of GFP⁺ wound cells as percentage of wound area. (D) Quantification of GFP⁺ fibroblasts as percentage of all fibroblasts on fluorescence-activated cell sorting (FACS; see FIG. 14F for FACS strategy). (E) Fluorescent histology of POD 14 wounds with immunofluorescent (IF) staining for indicated fibroblast subtype markers (yellow fluorescent signal). (F) Quantification of percentage of GFP⁺ wound cells colocalizing with given fibroblast subtype marker by area. (G) Schematic of Adipoq^{Cre-ERT}; R26^{mTmG}; R26^{Awai} wounding experiments for adipocyte ablation. (H) Hematoxylin and eosin (H&E) staining (top) and dermal thickness measurements (bottom) of skin (UW) and POD 14 wounds treated with PBS (control) or DT (to ablate adipocytes). Yellow dotted lines show dermal thickness in representative sections. (I) Picrosirius red histology (top) and uniform manifold approximation and projection (UMAP) mapping of quantified ECM ultrastructure parameters (bottom; each dot represents one histology image) for PBS- and DT-treated skin and wounds. (B, E) DAPI (4′,6-diamidino-2-phenylindole), nuclear counterstain (blue fluorescent signal); mTmG, Tomato⁺ cells (red fluorescent signal; background color of non-Cre-expressing cells in mTmG construct); Adipoq, GFP⁺ cells (green fluorescent signal; Adipoq^{Cre} lineage-positive cells). (C, D, F, H) Data shown as mean±standard deviation (S.D.). *P≤0.05, **P≤0.01, ****P≤0.0001. Scale bars, 500 μm (B), 25 μm (E), 150 μm (H), 20 μm (I).

FIG. 2: Clonal and single-cell transcriptomic analysis reveal differentiation dynamics of adipocyte-derived fibroblasts. (A) Schematic of Adipoq^{Cre-ERT}; R26^{VT2/GK3} (Rainbow) mouse wounding experiments for clonal analysis of adipocyte-derived cells. (B) Fluorescent histology with Col 1 IF staining (white signal) of Rainbow skin and postoperative day 14 (POD 14) wounds. eGFP, enhanced green fluorescent protein (green signal; Cre⁻/non-adipocyte lineage-derived cells in Rainbow construct); mOrange/mCerulean/mCherry, membrane-bound orange/cerulean/cherry fluorescent proteins (orange, blue, and red signals, respectively; collectively represent Cre⁺/adipocyte lineage-derived cells in Rainbow construct). Scale bar, 25 μm. (C) Quantification of wound area percent coverage (top) and percentage of Col1⁺ fibroblast wound area (bottom) by Rainbow clone color in unwounded skin versus POD 14 wounds. (D) Schematic of Rainbow construct for analysis of adipocytes and their derivatives (left) and experimental approach for FACS-isolating and separately sequencing adipocyte-derived (mOrange⁺, mCerulean⁺, and mCherry⁺) and non-adipocyte-derived (eGFP⁺) fibroblasts (right). (E) Top, UMAP of Rainbow fibroblast scRNA-seq data, color coded by Seurat cluster (top left; overlaid Roman numerals I-V indicate Seurat cluster identity) or Rainbow clone color (top right). Bottom, relative representation of adipocyte-derived (mOrange, mCerulean, or mCherry⁺; collectively, white) versus non-adipocyte-derived (eGFP⁺; green) cells in each Seurat cluster. (F) Heatmap showing top differentially expressed genes for each Seurat fibroblast cluster. (G) Violin plots showing expression of genes characteristic to fibroblasts (top) or adipocytes (bottom) in fibroblasts of each Rainbow clone color. (H) Fibroblast scRNA-seq UMAP colored by expression level for known mechanical signaling genes. (I) Top GO pathway analysis results for genes characteristic to cells in Seurat cluster III (fibroblast cluster containing almost exclusively adipocyte-derived cells). (J) CytoTRACE analysis showing predicted order (differentiation potential scores; left) and genes with strongest positive and negative correlation with these scores (right). (K) Pseudotime analysis of Rainbow fibroblast scRNA-seq data; plots colored by relative pseudotime value (left) and Seurat cluster identity (right). Red point within cluster III (left plot) indicates root for pseudotime calculations. (L) Expression of mechanical signaling genes across pseudotime. Overlaid lines represent regression fit to gene expression of cells over pseudotime; colors indicate Seurat cluster identity of each cell. (M) Schematic depicting proposed identities of adipocyte-derived (red, orange, or blue) and non-adipocyte-derived (green) wound fibroblasts based on scRNA-seq analysis. We propose that Seurat cluster III represents a “mechanically naïve” population of ADFs that expresses lower levels of mechanical signaling genes and is transcriptomically more similar to adipocytes, whereas the other Seurat clusters (I, II, IV, and V) represent more typical “mechanically sensitive” wound fibroblasts, comprising both ADFs and non-adipocyte-derived wound fibroblasts, which highly express mechanical activation and fibrosis genes. (E, H, J, K) Dotted circled regions indicate Seurat cluster III. (C) Data shown as mean±standard deviation (S.D.).

FIG. 3: Adipocytes transition to fibroblasts in response to mechanical stimuli in vitro and in vivo. (A) Top, schematic of mouse adipocyte culture and mechanomodulation experiments. Bottom left, immunofluorescence (IF) staining of mouse adipocytes cultured with or without mechanical stretch, in the presence of indicated mechanosignaling inhibitors (Piezo1 inhibitor [P1i], Piezo2 inhibitor [P2i], YAP inhibitor [YAPi], and FAK inhibitor [FAKi]) or no inhibitor (no treatment, NT), with IF staining for adiponectin (green-fluorescent signal) and Col1 (red signal). Bottom right, quantification of adiponectin and Col 1 expression from IF staining. (B) Top left, schematic of mouse hypertrophic scarring (HTS) model experiments with incisional wounding and mechanical distraction (stretching). Right panels: gross photographs (top row), H&E histology (second row; black dotted regions show wounded/stretched region of skin), fluorescent histology (third through fifth rows), and IF quantification (bottom row) of experimental conditions in mouse HTS model. Fluorescent histology shows Tomato background fluorescence (mTmG, red signal) and GFP from Adipoq lineage-derived cells (Adipoq, green signal) with IF staining for Col 1 (third row), Piezo1 (fourth row), or Piezo2 (fifth row; all IF staining, white signal). Bottom, quantification of ADFs (left panel) and Col 1, Piezo1, and Piezo2 expression (right three panels; *P≤0.05 vs. all other conditions) from IF staining. (C) Top left, whole mount histology of human adipose tissue with IF staining for Piezo1 (red signal), Piezo2 (green), and adiponectin (white). Top right, schematic of human adipocyte culture and mechanomodulation experiments. Bottom, as in (A) bottom, but with human adipocytes. (D) Top, schematic of human adipocyte culture to evaluate cell calcium content. Bottom left, IF staining of human adipocytes cultured in indicated conditions showing calcium staining (green signal). Bottom right, quantification of calcium staining. (E) Schematic (top), IF staining (as in (A) and (C); bottom left), and IF quantification (bottom right) of human adipocyte culture with Yoda1 (Piezo1 agonist). (F) As in (E), but with Piezo1 (P1 shRNA), Piezo2 (P2 shRNA), or scrambled shRNA (Scrambled).

(A-F) Data shown as mean±S.D. (B, D) *P≤0.05, **P≤0.01, ****P≤0.0001. Scale bars, 100 μm (A), 150 μm (B, second row), 200 μm (B, third through fifth rows), 100 μm (C-F).

FIG. 4: Adipocyte-targeted mechanosignaling blockade in wounds via small molecule inhibition and genetic knockout of Piezo1 (P1i) and Piezo2 (P2i) reduces scarring and fibrosis. (A) Schematic of Adipoq^{Cre-ERT}; R26^{VT2/GK3} wounding with small molecule P1i or P2i treatment. (B) Gross photos (top row; paired photos of top [left image] and underside [right image] of wounds) and H&E histology (middle row) of wounds treated with P1i, P2i, or control (PBS). Blue outlined regions and red arrows, fat accumulation; yellow arrows, regenerating secondary elements (e.g., hair follicles [HFs]). Bottom row, quantification of dermal thickness (left), number of HFs in wounded area (middle), or adipose coverage as percent of wound area (right) for each treatment condition. (C) Picrosirius red histology (top) and UMAP of quantified extracellular matrix (ECM) ultrastructure parameters (bottom; each dot represents one histology image) for indicated conditions. (D) Quantification of percentage of fibroblasts (top) or lipofibroblasts (bottom) that are Adipoq lineage-derived (eGFP and mOrange⁺, mCerulean⁺, or mCherry⁺) by fluorescence-activated cell sorting (FACS). (E) Left, fluorescent histology of wounds showing Rainbow clone colors (top; eGFP, green signal; mOrange, orange signal; mCerulean, blue signal; mCherry, red signal) or Col 1 immunofluorescence (IF) staining (bottom, red signal). Right, quantified percent wound area coverage by adipocyte-derived clones (top) and Col 1 expression by IF (bottom). (F) Left, Oil Red O staining (red) for lipid/oil glands (left); right, quantification of red staining density. (G) Young's modulus, calculated from tensile strength testing, by wound condition. (H) Schematic depicting mouse wounding for transgenic, adipocyte-targeted KO of mechanosignaling genes. (I-N) As in (B-G), but with genetic P1 or P2 Knockout out (KO) versus control (P1/P2 wildtype).

(B, D-G, I, K-N) Data shown as mean±S.D. *P≤0.05, **P≤0.01, ***P≤0.001, ****P≤0.0001. Scale bars, 250 μm (B, I), 20 μm (C, J), 25 μm (E, F, L, M).

FIG. 5: Adipocyte mechanosignaling (Piezo) blockade influences differentiation dynamics of “mechanically naïve” versus “mechanically activated” adipocyte-derived fibroblasts. (A) Schematic of wounding experiments with Piezo1 inhibitor (P1i), Piezo2 inhibitor (P2i), or Piezo1 knockout (P1 KO) and scRNA-seq analysis. (B) UMAP of scRNA-seq data from all wound cells, colored by cell type. Black dotted region indicates cells transcriptomically classified as fibroblasts (i.e., in silico selection) that were used for downstream analysis. (C) UMAP plots of fibroblasts colored by either Seurat cluster (0-5; left) or experimental condition (right). (D) Heatmap showing top differentially expressed genes for each Seurat fibroblast cluster. (E) Top, schematic depicting approach for projecting the six clusters derived from the current dataset (left) on the original five scRNA-seq clusters from FIG. 2 (right). Center, label transfer projection of FIG. 5C fibroblast subpopulations onto FIG. 2 embedding. Colored circles highlight the spatial locations of FIG. 5C subpopulations. Bottom, summary of correlations between fibroblast clusters from FIG. 2 to FIG. 5C; arrows indicate Seurat cluster from original dataset that most closely corresponds to each cluster in new dataset (heavy pink arrow indicates correlation between FIG. 2 cluster III and FIG. 5 cluster 5). (F) Gene Ontology (GO) pathway analysis for indicated Seurat clusters in new (all wound conditions) scRNA-seq dataset. (G) Relative representation of fibroblasts belonging to clusters 0-4 versus cluster 5 from each experimental condition. (H) Quantification of percentage of all sequenced wound cells from each condition that were considered fibroblasts (top) or adipocytes (bottom) based on transcriptomic profiles. (I) scVelo heatmap highlighting genes with high correlation with velocity pseudotime, indexed by Seurat cluster. (J) Left, pseudotime analysis of fibroblasts from all wound conditions, with plots colored by relative pseudotime value (top) and Seurat cluster identity (bottom). Right, expression of mechanical signaling genes across pseudotime. (K) Schematic depicting proposed identities of “mechanically naïve” (cluster 5) versus typical “mechanically sensitive” (clusters 0-4) wound fibroblasts based on scRNA-seq analysis.

FIG. 6: Piezo1 inhibition during wound remodeling reduces fibrosis in existing mouse scars. (A) Schematic, treatment of existing scars with Piezo1 inhibitor (P1i) during early remodeling phase (POD 30) with harvest at POD 60. (B) Gross photographs of wounds at POD 0, 30, 60. (C) Schematic, treatment of existing scars with Piezo1 inhibitor (P1i) during mid remodeling phase at POD 75 with harvest at POD 105 and late remodeling at POD 120 with harvest at POD 150. (D) Gross photographs of wounds at POD 0, 75 and 105 (left) and POD 0, 120, and 150 (right) following PBS (top) or P1i treatment (bottom). (E) H&E (top) and trichrome (bottom) staining of POD 60 wounds treated with P1i (left) or control (untreated; right) at POD 30. (F) Top, picrosirius red staining of unwounded (UW) skin and POD 60 wounds treated at POD 30 with P1i or PBS (control). Bottom, UMAP of quantified extracellular matrix (ECM) ultrastructure parameters based on picrosirius red histology (each dot represents one histologic image). Data shown as mean±S.D. *P≤0.05. Scale bars, 3 mm (A), 150 μm (E), 20 μm (F).

FIG. 7: Visium analysis reveals that piezo inhibition during wound healing alters the spatial transcriptional. (A) Schematic for generating spatial transcriptomic data from splinted excisional wounds using the 10× Genomic protocol. (B) [I-IV] Delineation of scar layers based on Seurat Clusters (top left) with UMAP plot (top right) and underlying tissue histology (bottom left) with UMAP plot (bottom right) showing that the three scar layers can easily be distinguished by their transcriptional programs. Arrows the same tissue sample just colored by either Seurat Clusters or histological layer. (C) Schematic showing the anchor-based integration of scRNA-seq populations (defined in FIG. 5) with Visium gene expression to project partial membership within each spot across all groups. (D) Spatial plots showing spatial expression of fibroblast cluster 0 (mechanically sensitive) and cluster 5 (mechanically naïve) in PBS (top) and P1i-treated wounds (bottom). (E) Differential interaction maps in P1i vs. PBS-treated wounds (left) and PBS-treated wounds vs. P2i treated wounds (right) at POD 14. (F) CytoTRACE analysis of PBS (top) and P1i-treated wounds (bottom).

FIG. 8: CODEX analysis reveals that Piezo inhibition during wound healing alters the spatial cross-talk between adipocytes and fibroblasts. (A) Schematic of the CODEX experiment strategy. (B) UMAP plot of CODEX data for all sequenced cells. (C) Representative images of identified UMAP CODEX clusters showing their spatial distribution in unwounded skin and PBS- and P1i/P2i-treated wounds. (D) Representative images of six selected CODEX markers in unwounded skin and PBS- and P1i/P2i-treated wounds. (E) Differential interaction maps in unwounded skin vs. PBS-treated wounds (top left), PBS-treated wounds vs. P1i treated wounds (top right), and PBS-treated vs P2i treated wounds (bottom). (F) Histograms of mechanosensitive protein module expression (PIEZO1, PIEZO2, YAP1, and PTK2 in adipocytes (left) and fibroblasts (right). (G) Heatmap of adipocyte-fibroblast interactions in all wounds. (H) Bar graph quantifying adipocyte cluster 1-fibroblast cluster 2 interactions in unwounded, PBS, and P1i/P2i treated wounds. (I) Bar graph quantifying adipocyte cluster 2-fibroblast cluster 2 interactions in unwounded, PBS, and P1i/P2i treated wounds. Scale bars, 50 μm (D).

FIG. 9: Visium analysis reveals that piezo inhibition during scar rescue alters the spatial transcriptional landscape. (A) Schematic for generating spatial transcriptomic data during scar rescue using the 10× Genomic protocol. (B) [I-VI] Top: Spatial plots (left) of scars colored by Seurat colors with UMAP (right). Middle: Spatial plots (left) of scars colored by tissue histology (left) with UMAP (right) showing that the three scar layers can easily be distinguished by their transcriptional programs. Bottom: Spatial plots (left) of scars colored by predicted cell types following anchor transfer from FIG. 5 (left) with UMAP (right). Bottom: Schematic showing the anchor-based integration of scRNA-seq populations (defined in FIG. 5) with Visium gene expression to project partial memberships within each spot across all groups. Arrows represent the same sample just colored by either UMAP or histological layer. (C) Spatial plot of adipocytes in P1i treated (top) and PBS treated (bottom) wounds at POD 60. (D) Differential interaction maps in P1i vs. PBS-treated wounds at POD 60.

FIG. 10: CODEX analysis reveals that piezo inhibition following scar rescue alters the spatial cross-talk between adipocytes and fibroblasts. (A) Schematic of the CODEX experiment. (B) UMAP plot of CODEX data for all sequenced cells. (C) Representative images of identified UMAP CODEX clusters showing their spatial distribution in PBS- and P1i-treated wounds. (D) Representative image of six selected CODEX markers in PBS (left)- and P1i-(right) treated wounds at POD 60 (top) and POD 150 (bottom). (E) Differential interaction maps in PBS-treated vs. P1i-treated wounds at POD 60 (top) and POD 150 (bottom). (F) Bar graph quantifying adipocyte cluster 2-epithelial cell interactions (left) and adipocyte cluster 1-smooth muscle cell interactions (right) in PBS and P1i treated wounds. (G) Histograms of mechanosensitive protein module expression (PIEZO1, PIEZO2, YAP1, and PTK2) in adipocytes (left) and fibroblasts (right). (H) Bar graphs quantifying fibroblast 1-adipocyte interactions (left) and fibroblast 5-adipocyte interactions (right) in all wounds. (I) Heatmap of adipocyte-fibroblast interactions in all wounds. Data shown as mean±S.D. *P≤0.05, ***P≤0.001. Scale bars, 180 μm (D).

FIG. 11: Piezo inhibition reduces scarring in a human skin xenograft wound model. (A) Schematic of human foreskin xenografting and wounding experiments. (B) Gross photos of healed postoperative day (POD) 14 xenograft wounds following treatment with Piezo1 inhibitor (P1i), Piezo2 inhibitor (P2i), or control (PBS). Blue outlined regions, xenograft; red dotted region, wound within xenograft. (C) Quantification of scar area as percentage of total graft area from gross images. (D) Schematic depiction (top row) and H&E histology (bottom three rows) xenograft wounds. Dotted lines on histology images indicate boundaries of regions identified in schematic (e.g., mouse unwounded [UW], human UW) in corresponding color font. (E) Quantification of xenograft wound dermal thickness from histology. (F) Top, picrosirius red staining of xenograft wounds. Bottom, UMAP of quantified extracellular matrix (ECM) ultrastructure parameters for indicated xenograft conditions. (G) IF staining for human-specific Col 1 (hCol1, top) or adiponectin (bottom; both, green signal) in indicated xenograft wound conditions. (H) Quantification of hCol1 and adiponectin expression in xenograft wounds from IF. (I) IF staining for human CK19 (hCK19, top) or human CD31 (hCD31, bottom; both, green signal) in indicated xenograft wound conditions. (J) Quantification of hCK19 and hCD31 expression in xenograft wounds from IF.

(C, E, H, J) Data shown as mean±S.D. *P≤0.05, ***P≤0.001. Scale bars, 500 μm (B, D), 25 μm (F), 50 μm (G, I).

FIG. 12: Piezo inhibition reduces scarring in a human skin xenograft wound model through altering the transcriptional profile of resident cells. (A) Schematic of the xenograft wounding model. (B) UMAP of scRNA-seq data from all wound cells, colored by Seurat cluster. (C) Heatmap showing top differentially expressed genes for each Seurat fibroblast cluster. (D) Relative representation of fibroblasts belonging to each Seurat cluster from each experimental condition. (E) Violin plots showing gene expression of Twist2, Wnt5a, Calu, and Sfrp2 among the Seurat clusters. (F) Immunostaining of Sfrp2, Twist2, Calu, and Wnt5a (green) with co expression of collagen type I (red) at Post-operative day 14 (POD 14) in wounded (left) and P1i treated wounds (right). (G) Gene Ontology (GO) pathway analysis for human cluster 3. (H) CytoTRACE analysis identified significant differences in the differentiation state of cluster 3 cells compared to other fibroblast clusters. (I) Pseudotime analysis of the fibroblast clusters. (Red dot shows the root point) (J) Schematic of the anchor transfer of human fibroblasts onto the mouse sorted fibroblasts (from FIG. 2) with arrows demonstrating the clusters that were transcriptionally similar. (K) Bar graph depicting the similarity of the mouse cluster 3 onto the human xenograft unwounded, wounded and P1i treated derived fibroblasts. (L) Bar graph depicting the similarity of the mouse cluster 3 onto the human xenograft fibroblasts derived Seurat clusters. Data shown as mean±S.D. *P≤0.05, ***P≤0.001. Scale bars, 250 μm (F).

FIG. 13: CODEX analysis reveals that piezo inhibition in human xenografts alter spatial cell cross-talk (A) Schematic of the CODEX experiment. (B) UMAP plot of CODEX data for all sequenced cells. (C) Representative images of identified UMAP CODEX clusters showing their spatial distribution in unwounded skin and PBS- and P1i-treated wounds. (D) Representative images of six selected CODEX markers in unwounded skin and PBS- and P1i-treated wounds. (E) Bar graphs quantifying adipocyte cluster 1 (left) and adipocyte cluster 4 (middle) proportions in unwounded skin and PBS- and P1i-treated wounds, with representative feature plot of adipocyte cluster 1 and cluster 4 (right). (F) Differential interaction maps in unwounded skin vs. PBS-treated wounds (left) and PBS-treated wounds vs. P1i-treated wounds (right). (G) Histograms of mechanosensitive protein module expression (PIEZO1, PIEZO2, YAP, and FAK) in adipocytes (left) and fibroblasts (right). (H) Bar graph of adipocyte subpopulation interactions with all fibroblasts (top) and heatmap of individual adipocyte-fibroblast interactions (bottom) in all wounds. (I) Correlation between fibroblast clusters identified by scRNA-seq and CODEX analyses (left). Feature plots showing that scRNA-seq cluster 3 fibroblasts mapped onto CODEX type 4 adipocytes (middle) and scRNA-seq cluster 0 fibroblasts mapped onto CODEX Type 1 adipocytes (right). Data shown as mean±S.D. *P≤0.05, ***P≤0.001. Scale bars, 150 μm left 50 μm right (D).

FIG. 14: Adipocyte transplantation and wounding experiments. A. Schematic of fluorescent (mTmG, Tomato red fluorescent protein [RFP]⁺) adipocyte transplantation and wounding experiments. B-C. Immunofluorescent (IF) histology of transplanted mTmG adipocytes in unwounded skin (B) and postoperative day (POD 14) wounds (C) with IF staining for fibroblast and adipocyte markers. mTmG, Tomato fluorescence from transplanted adipocytes (red fluorescent signal); IF staining for adiponectin (yellow signal) and Col1 (green signal). Dotted orange arrows, Tomato⁺ cells; solid white arrows, Adipoq⁺Col1⁺Tomato⁺ cells; dotted white arrows, Adipoq⁻Col1⁺Tomato⁺ cells. Scale bar, 50 μm. D. Quantification of transplanted mTmG cells also expressing Col1 per high-powered field (HPF). E. Top: Breeding scheme of Adipoq^{Cre-ERT}; R26^{mTmG} mice. Bottom: Schematic of Adipoq^{Cre-ERT}; R26^{mTmG} wounding experiments for lineage tracing of adipocyte-derived cells. F. Fluorescence-activated cell sorting (FACS) strategy for sorting GFP⁺ (adipocyte-derived; GFP⁺ gate) versus Tomato⁺ (mTmG, non-adipocyte-derived; PE⁺ gate) fibroblasts (Lin; PB gate) with representative plots for unwounded (UW) and POD 14 wound sorts. G. FACS strategy for sorting lipo-(Sca1⁺; BV 605⁺ gate), reticular (Dlk1⁺; APC⁺ gate), and papillary dermal (CD26⁺; PerCP Cy5.5⁺ gate) ADF (Lin⁻GFP⁺) subtypes. H. Quantified percentage of wound ADFs expressing markers of each fibroblast subtype by FACS.

D, H. Data shown as mean±S.D. *P≤0.05, ***P≤0.001.

FIG. 15: Immunofluorescent analysis of ADF marker expression in Adipoq^{Cre-ERT}; R26^{mTmG} skin and wounds. A. Immunofluorescent (IF) staining of unwounded (UW) skin and postoperative day (POD) 14 wounds for CD31 (endothelial cell marker; purple fluorescent signal). B. IF staining of UW skin and POD 14 wounds for CD8 and CD4 (T cell markers) and F480 (macrophage marker; all, purple signal). C. IF staining of POD 14 wounds showing that ADFs gain expression of indicated fibroblast markers (top row), lose adipocyte markers (second row), and localize with known fibrotic signaling markers (bottom row; all IF, yellow signal). A-C. DAPI, nuclear counterstain (blue signal); mTmG, Tomato⁺ (Adipoq lineage-negative) cells (red signal); Adiponectin, GFP⁺ (Adipoq lineage-positive) cells (green signal). Scale bars, 25 μm.

FIG. 16: Analysis of adipocyte ablation in Adipoq^{Cre-ERT}; R26^{mTmG}; R26^{Awai} wounds. A. Fluorescence-activated cell sorting (FACS) strategy for quantifying GFP⁺ (adipocyte-derived) wound cells with representative plots for diphtheria toxin (DT)- and PBS-treated wounds showing that GFP⁺ cells are ablated with DT treatment. B. Masson's trichrome staining (top) and quantification of average blue (connective tissue) staining density (bottom) for PBS- and DT-treated unwounded (UW) skin and POD 14 wounds. C. Top, fluorescent histology of POD 14 PBS- and DT-treated wounds with IF staining for Col 1 or α-smooth muscle actin (α-SMA) (yellow). DAPI, nuclear counterstain (blue signal); mTmG, Tomato⁺ (Adipoq lineage-negative) cells (red signal); Adipoq, GFP⁺ (Adipoq lineage-positive) cells (green signal). Bottom, quantification of Col 1 and α-SMA expression from IF. D. Young's modulus calculated from tensile strength testing of PBS- and DT-treated wounds or UW skin.

B-D. Data shown as mean±S.D. *P≤0.05. Scale bars, 25 μm.

FIG. 17: EdU analysis of cell proliferation in Adipoq^{Cre-ERT}; R26^{mTmG} wounds. A. Labeling of proliferating cells in unwounded (UW) skin and wounds at indicated timepoints using EdU (purple signal); DAPI, nuclear counterstain (blue signal); mTmG, Tomato⁺ (Adipoq lineage-negative) cells (red signal); Adipoq, GFP⁺ (Adipoq lineage-positive) cells (green signal). Scale bars, 100 μm. B. Quantification of EdU signal colocalization with adipocytes (GFP⁺Col1⁻) and ADFs (GFP⁺Col1⁺) from histology. C. Quantification of percentage of non-adipocyte-derived (GFP⁻) and adipocyte-derived (GFP⁺) fibroblasts (Lin) positive for EdU incorporation by fluorescence-activated cell sorting (FACS).

B-C. Data shown as mean±S.D.

FIG. 18: Analysis of Rainbow ADF scRNA-seq data. A. Fluorescence-activated cell sorting (FACS) strategy for sorting fibroblasts (Lin) of each Rainbow reporter color (eGFP, mCerulean, mCherry, or mOrange). B. Fluorescent histology of non-tamoxifen-induced Rainbow control specimens showing only eGFP expression (green signal) and absence of other Rainbow clone colors (mOrange, mCerulean, or mCherry; orange, blue, or red signal, respectively) that would indicate nonspecific recombination in the absence of tamoxifen induction. Scale bar, 25 μm. C. UMAP of Rainbow scRNA-seq fibroblasts colored by cell type (SingleR) demonstrating that all cells had a fibroblast transcriptomic identity. D. Quantification of relative representation of cells belonging to each Seurat fibroblast cluster (I-V) by Rainbow clone color. E. Violin plots showing expression of known fibroblast (top) and adipocyte (bottom) genes for cells of each Rainbow color (eGFP, non-adipocyte lineage-derived; mCerulean, mOrange, and mCherry, adipocyte-derived). F. Expression of mechanical signaling genes by Seurat cluster identity. G. GO pathway analysis for Seurat fibroblast clusters I, II, IV, and V. H. GeneTrail analysis for Seurat fibroblast clusters I-V showing comparable enrichment for fibrosis-related processes in clusters I, II, IV, and V. I. Gene Ontology (GO) pathway analysis for cells based on Rainbow color. J. Rainbow fibroblast UMAP showing anchor-based label transfer mapping of embedded cells from “mechanosensitive” and “non-mechanosensitive” fibroblast populations previously defined by Foster et al.²⁵ K. Top, clustergram of pseudotime gene modules, with gene modules 1-4 indicated in red boxes in top panel. Bottom, expression scores of pseudotime modules 1-4 representing trajectories starting from cluster III. L. Expression of genes from pseudotime gene modules 1-4 across pseudotime (indicated in red boxes in top panel K). Overlaid lines represent regression fit to gene expression of cells over pseudotime; colors indicate Seurat cluster identity of each cell. M. scVelo analysis of cells along the first two principal components, demonstrating differentiation trajectory stemming from Seurat cluster III (black circle). N. scVelo heatmap highlighting genes with high correlation with velocity pseudotime, indexed by Seurat clusters. O. Top and bottom left, scVelo analysis of root and end point cells (black and red circles highlight root and end point cells, respectively). Top right, scVelo analysis of velocity pseudotime showing trajectory starting from cluster III (red circle). Bottom right, scVelo analysis of velocity length showing trajectory starting from cluster III (red circle). P. Gene-level scVelo analysis of specific genes of interest, depicting differences between spliced and unspliced RNA counts.

FIG. 19: Analysis of mechanosignaling activity and inhibition in mouse adipocytes in vitro and in vivo. A. Immunofluorescence (IF) staining of mouse adipocytes cultured with mechanical stretching, with or without (no treatment, NT) indicated mechanosignaling inhibitors. IF staining for adiponectin (green fluorescent signal), Col 1 (red signal), and indicated mechanosignaling markers (white signal). B. Quantification of adiponectin and Col 1 expression by quantitative reverse transcription polymerase chain reaction (RT-qPCR). C. Quantification of percent of cultured cells that are fibroblasts (Lin) or adipocytes (LipidTox⁺) by FACS. D. Enzyme-linked immunosorbent assay (ELISA) quantification of collagen (left) or adiponectin (right) protein expression in cultured cells. E. Lipid Tox staining of mouse adipocytes with stretch (top), unstretched (middle), and with P1i inhibitor (bottom). F. Top, fluorescent histology of mouse hypertrophic scarring (HTS) model wounds and skin, indicated conditions; mTmG, Tomato⁺ (Adipoq lineage-negative) cells (red signal); Adiponectin, GFP⁺ (Adipoq lineage-positive) cells (green signal); IF staining for FAK (first row) or YAP (second row; both, white signal). Bottom, quantification of FAK (left) and YAP (right) expression from IF staining. G. Picrosirius red staining of skin and wounds from mouse HTS model experimental conditions (surrounding panels) and UMAP of quantified extracellular matrix (ECM) ultrastructure parameters based on picrosirius red histology (central panel; each dot represents one histologic image). H. IF staining of mouse adipocytes cultured under varying concentrations of mechanosignaling inhibitors (see Methods for specific concentrations) or NT control, with IF staining for adiponectin (green signal), Col 1 (red signal), Ki67 (gray signal), perilipin (green signal), Piezo1 (red signal), and Piezo2 (red signal) as indicated. I. Percentage of all cells positive for EdU, annexin V (apoptosis marker), or DAPI (dead cells) by FACS of mouse adipocytes treated with indicated varying concentrations of Piezo1 inhibitor (P1i). None of the markers' expression was significantly different between any of the inhibitor concentrations or compared to control (P>0.05).

B-E, H. Data shown as mean±S.D. B-E. *P≤0.05 (vs. all other conditions). Scale bars, 10 μm (A), 100 μm (E), 150 μm (F), 100 μm (G).

FIG. 20: Analysis of mechanosignaling activity and inhibition in human adipocytes in vitro. A. Left, immunofluorescence (IF) staining of human adipose tissue with IF staining for indicated mechanosignaling markers (green signal). Right, quantification of expression of each marker from IF. B-E. As in 19A-D, but with human adipocytes. F, G. As in C, D, but with or without Yoda1 (Piezo1 agonist) treatment. H. IF staining of cultured human adipocytes treated with indicated shRNA with IF staining for adiponectin (green signal), Col 1 (red signal), and Piezo1 or Piezo2 as indicated (white signal). I, J. As in C, D, but with indicated shRNA treatments.

A, C-G, I-J. Data shown as mean±S.D. C-G, I-J. *P≤0.05, **P≤0.01, ***P≤0.001. P-value reflects pairwise comparison between indicated conditions, or comparison of one condition vs. all other conditions when specific pairwise comparison not indicated. Scale bars, 100 μm (A, B, H).

FIG. 21: Piezo1 and Piezo2 lineage tracing in wounds. A. Schematic of Piezo 1^{t-dT} and Piezo2^{EGFP-IRES-Cre} wounding experiments to genetically trace Piezo1 and Piezo2 lineage-positive cells in wounds. B. Fluorescent histology of unwounded (UW) skin (top) and POD 14 wounds (bottom left) with immunofluorescence (IF) staining for fibroblast and adipocyte markers. First column: Piezo1, tdTomato signal labeling Piezo1 lineage-derived cells (red signal); IF for Col 1 or α-smooth muscle actin (a-SMA) (green signal), adiponectin or perilipin (gray signal). Second column: Piezo2, EGFP signal labeling Piezo2 lineage-derived cells (green signal); IF for Col 1 or α-SMA (red signal), adiponectin or perilipin (gray signal). Bottom right, quantification of Piezo1 (tdTomato⁺) and Piezo2 (EGFP⁺) lineage-derived cells in respective lineage-tracing models by fluorescent signal on histology, UW skin vs. POD 14 wounds. C. Fluorescence-activated cell sorting (FACS) quantification of percentage of cells of Piezo1 or Piezo2 lineage origin based on tdTomato or EGFP expression, respectively. D. Left panels, RNAscope in situ hybridization for Piezo1 (first column) and Piezo2 (second column; both, pink signal). Top right, higher-power zoom of Piezo1 (left) and Piezo2 (right) RNAscope. Red arrows indicate positively stained cells. Right bottom, quantification of Piezo1⁺ and Piezo2⁺ cells per HPF from RNAscope.

B-D. Data shown as mean±S.D. B, C. *P≤0.05. Scale bars, 50 μm (B), 100 μm (D, left panels), and 20 μm (D, right panels).

FIG. 22: Analysis of wounds treated with small molecule inhibitors of Piezo1 and Piezo2. A. Wound curve showing percent of original wound area re-epithelialized at indicated timepoints in wounds treated with Piezo1 inhibitor (P1i), Piezo2 inhibitor (P2i), or PBS (control). B. Top, fluorescent histology of Rainbow wounds showing Rainbow clone colors (first row; eGFP, green signal; mOrange, orange signal; mCerulean, blue signal; mCherry, red signal) or immunofluorescence (IF) staining for a-smooth muscle actin (α-SMA) (second row), CK14 (third row), or CK19 (fourth row; all IF, red signal, not consecutive sections). Bottom, quantification of expression of indicated markers from IF. *P≤0.05 vs. all other conditions. C, D. H&E staining (left) and Masson's trichrome staining (right) of POD 14 (C) and POD 30 (D) wounds with indicated treatments (P2iLD; P2i Low dose, P2iHD; P2i high dose, P1iLD; P1iLow dose, P1iHD; P1iLow dose). (E) Gross photos of wounds at POD 30 treated with PBS, P1I or P2I.

A, B. Data shown as mean±S.D. Scale bars, 25 μm (B), 500 μm (C, D), 6 mm (E).

FIG. 23: Analysis of wounds treated with adipocyte-targeted genetic knockout of mechanosignaling genes. A. Wound curve showing percent of original wound area re-epithelialized at indicated timepoints in wounds with adipocyte-targeted Piezo1 (P1), Piezo2 (P2), YAP, or FAK knockout (KO). B. Histology of YAP (left) and FAK (right) KO wounds, each showing H&E (top left), Masson's trichrome (bottom left), and Oil Red O (bottom right) staining. C. H&E (top) and Masson's trichrome (bottom) staining of indicated wound conditions at POD 30. D. Fluorescent histology of Rainbow wounds with P1, P2, YAP, or FAK KO or control at POD 14, showing Rainbow clone colors (first row; eGFP, green signal; mOrange, orange signal; mCerulean, blue signal; mCherry, red signal) or IF staining for α-SMA (second row), CK14 (third row), CK19 (fourth row, or Col1 (bottom row right; all IF, red signal, not consecutive sections). Bottom left, quantification of expression of indicated markers by IF. E. Top, picrosirius red staining of unwounded (UW) skin and control, FAK KO, and YAP KO wounds at POD 14; bottom, UMAP of quantified extracellular matrix (ECM) ultrastructure parameters based on picrosirius red histology (each dot represents one histologic image). F. Young's modulus, calculated from tensile strength testing, by wound condition.

A, D, F. Data shown as mean±S.D. D, F. *P≤0.05. P-value reflects pairwise comparison between indicated conditions, or comparison of one condition vs. all other conditions when specific pairwise comparison not indicated. Scale bars, 500 μm (B, H&E and trichrome), 25 μm (B, Oil Red O), 500 μm (C), 25 μm (D), 20 μm (E).

FIG. 24: scRNA-seq analysis of wounds with and without Piezo inhibition. A. UMAP of all wound cells colored by Seurat cluster (dataset from FIG. 5). B. Violin plots showing expression of known fibroblast (left) and adipocyte (right) genes for cells of each new fibroblast Seurat cluster (0-5). C. Violin plots showing expression of indicated characteristic adipocyte genes by cell type within scRNA-seq dataset. D. Gene ontology (GO) pathway analysis for FIG. 5 Seurat fibroblast clusters 1, 2, 3, and 4. E. Quantification of relative representation of cells belonging to each new Seurat fibroblast cluster (0-5) by experimental condition. F. Fibroblast UMAP colored by expression of select mechanosignaling genes. G. scVelo analysis of root cells showing trajectory starting at cluster 5 (black circle). H. Left, scVelo velocity length analysis showing the trajectory starting at cluster 5 (red circle). Right, scVelo velocity pseudotime showing the trajectory starting at cluster 5 (red circle). I. Gene-level scVelo analysis of specific genes of interest, depicting differences between spliced and unspliced RNA transcripts. J. Left, clustergram of pseudotime gene modules, with gene modules 1-4 indicated in red boxes. Right, expression scores of pseudotime modules 1-4 representing trajectories starting from cluster 5. K. Expression of genes from pseudotime gene modules 1-4 (indicated in red boxes in left panel of J) across pseudotime. Overlaid lines represent regression fit to gene expression of cells over pseudotime; colors indicate Seurat cluster identity of each cell.

FIG. 25: CellChat analysis of cell-cell interactions by wound condition from scRNA-seq dataset. A. Interaction weight/strength of cell-cell interactions between all sequenced cells in unwounded skin and wounds at POD 14 (control, Piezo1 inhibitor [P1i], Piezo2 inhibitor [P2i], and P1 knockout [KO] wounds). B. Circle plot of cell-cell interactions for each fibroblast cluster. C. River plot of outgoing communication from cells in unwounded (UW) and wounded skin at POD 14. D. Heatmap illustrating cell-cell interactions in P1i compared to control wound cells (blue shades, decreased cell-cell signaling in P1i compared to control wounds; red, increased cell-cell signaling in P1i compared to control wounds). Purple boxes highlight increased overall interactions between cluster 5 fibroblasts and other cell types. E. Bar chart showing differential interaction strength between control vs. P1i wound cells. F. Relative (control vs. P1i, normalized to add to 1) calculated information flow for indicated genes for all cell interactions following control vs. P1i treatment. G Number of interactions between adipocytes and cluster 5 fibroblasts in control and P1i treated wounds.

FIG. 26: Analysis of Piezo1 inhibition during scar rescue. A. H&E (top row) and Masson Trichome staining (bottom row) of P1i and PBS treated wounds at POD 105 (top) and POD 150 (bottom). B. Representative Picrosirius red staining image (right) and associated UMAP (left) following PBS or P1i treatment at POD 105. C. Representative Picrosirius red staining image (right) and associated UMAP (left) following PBS or P1i treatment at POD 150. D. Fluorescent histology of scars at POD 60 following PBS or P1i treatment showing immunofluorescence (IF) staining for Collagen type I (Col1) (top row) and Piezo1 (bottom row). E. Quantification of expression of indicated markers from IF. *P≤0.05 vs. all other conditions. F. Quantification of GFP cells at POD 60 scars treated with either P1i or PBS by flow cytometry. G. Fluorescent histology of scars at POD 105 following PBS or P1i treatment showing immunofluorescence (IF) staining for Collagen type I (Col1) (top row) and Piezo1 (bottom row) at POD 105. H. Fluorescent histology of scars at POD 150 following PBS or P1i treatment showing immunofluorescence (IF) staining for Collagen type I (Col1) (top row) and Piezo1 (bottom row) at POD 150. Scale bars, 250 μm (A, D, G, H), 25 μm (C).

FIG. 27: Analysis of Piezo1 inhibition during scar rescue. A. Gross histology scars at POD 60 following PBS or B. P1i treatment (red dotted arrows illustrating the scars). All gross histology of wounds at POD 60 are shown. Scale bar=3 mm.

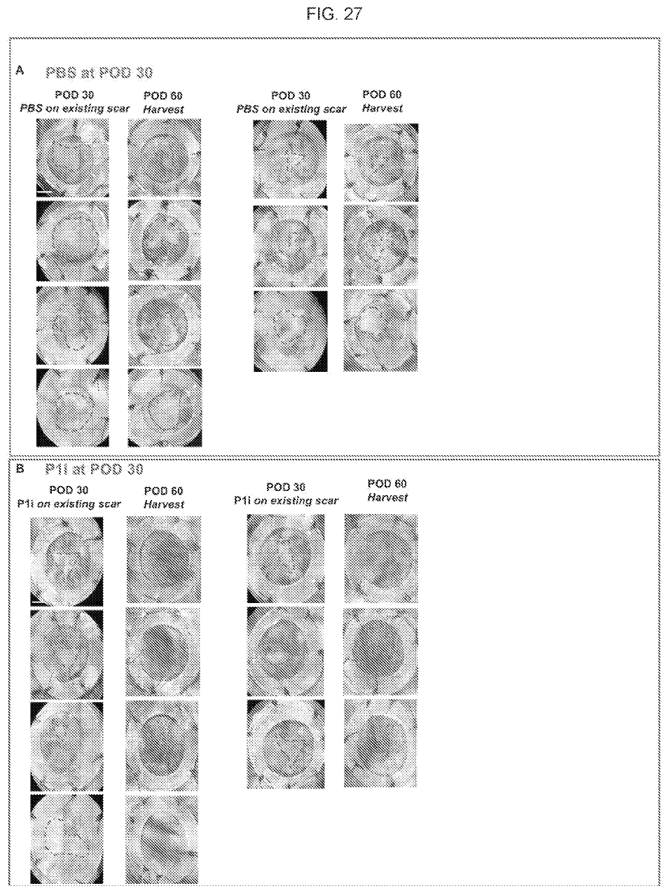

FIG. 28: Analysis of Piezo1 inhibition during scar rescue. A. Gross histology scars at POD 105 following PBS or B. P1i treatment (red dotted arrows illustrating the scars). All gross histology of wounds at POD 105 are shown. Scale bar=3 mm.

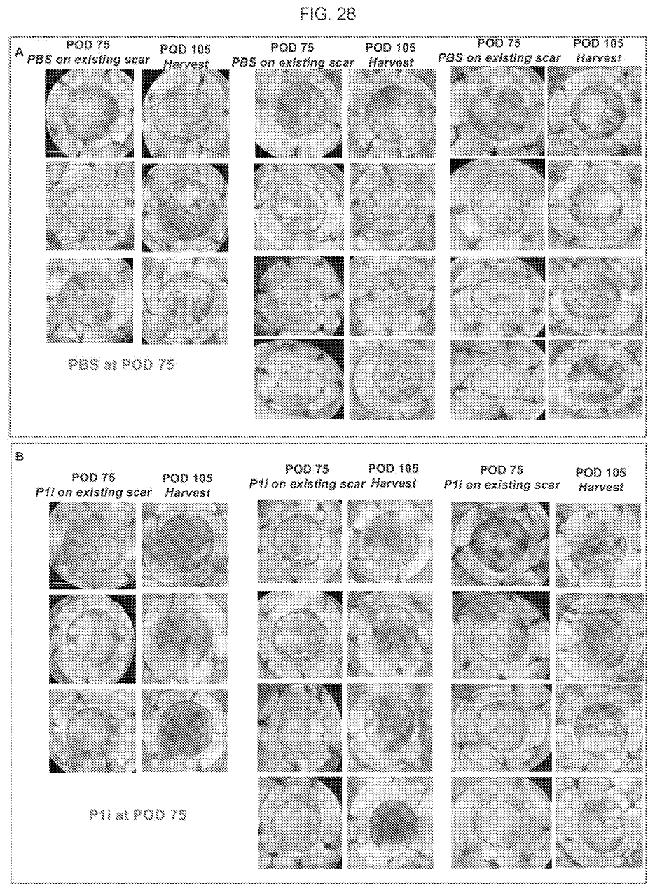

FIG. 29: Analysis of Piezo1 inhibition during scar rescue. A. Gross histology scars at POD 150 following PBS or B. P1i treatment (red dotted arrows illustrating the scars). All gross histology of wounds at POD 150 are shown. Scale bar=3 mm.

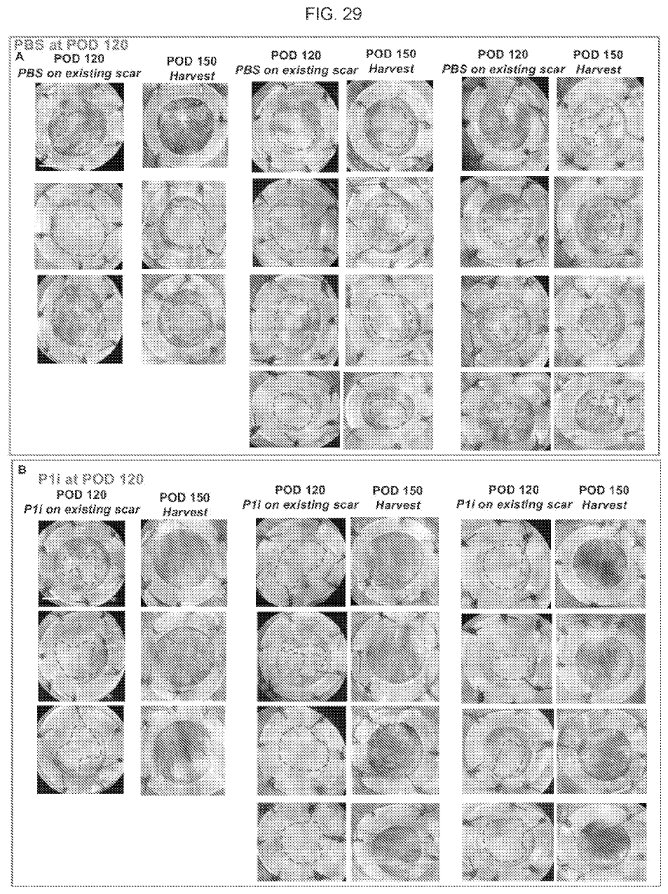

FIG. 30: Analysis of YAPi inhibition during scar rescue. A. Schematic showing YAPi or PBS of scars at POD 30 and harvested at POD 60. B. Gross histology scars at POD 60 following PBS or C. YAPi treatment (red dotted arrows illustrating the scars). Scale bar=3 mm.

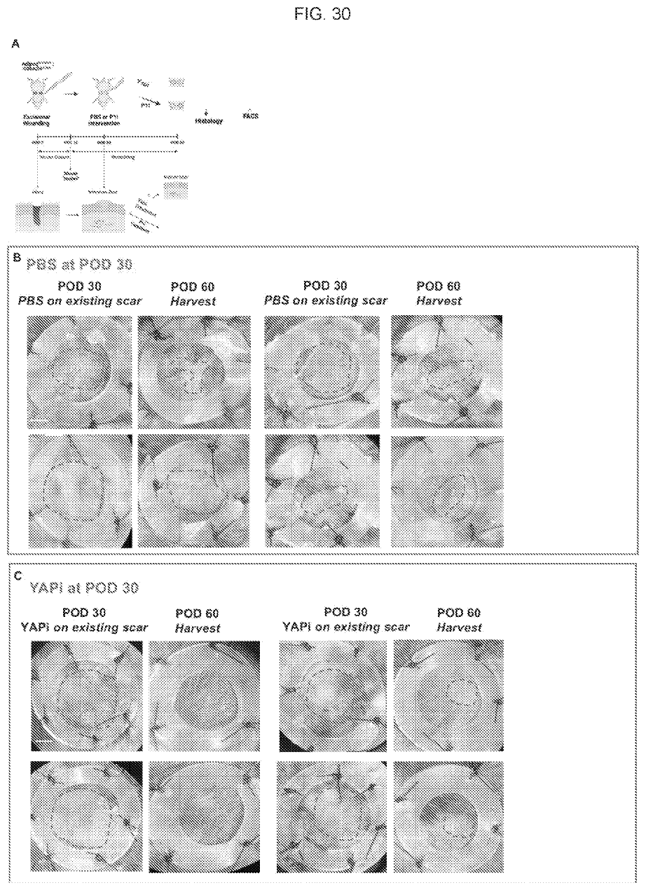

FIG. 31: VISIUM analysis at POD 14. A. Spatial plots of epithelial (Krt14), immune (Ptprc), fibroblast (Col1a1) and adipocyte (Adipoq) cell markers at POD 14 following PBS treatment. B. Spatial plots of mechanical markers (Piezo1, Piezo2, Ptk2, and Yapi) at POD 14 following PBS treatment (left) and P1i treatment (right).

FIG. 32: VISIUM analysis at POD 7. A. Spatial plots of POD 7 PBS (left) or P1i treated (right) wounds colored by Seurat clusters (top row) with associated UMAPs (left). Spatial plots of POD 7 PBS (left) or P1i treated (right) wounds colored by epithelial, dermal and hypodermis category (right) with associated UMAP (left). B. Spatial plots of mechanical markers at POD 7 (Piezo1, Piezo2, Ptk2, and Yapi) following PBS treatment (left column) and P1i treatment (right column).

FIG. 33: VISIUM analysis at POD 14. A. Spatial plots of spatially variable features at POD 14 following PBS treatment (top right), unwounded (top left), following P1i treatment (bottom left) and P2i treatment (bottom right). B. Spatial plots of cell types maximally predicted from scRNAseq FIG. 5 data.

FIG. 34: CODEX analysis at POD 14. A. Representative UMAP images of CD31 (top), CD86 (middle) and Desmin (bottom) of protein expression. B. Differential interaction maps in unwounded skin vs. P1i-treated wounds (top left), P1i-treated wounds vs. P2i treated wounds (top right), and unwounded vs P2i treated wounds (bottom). C. Histogram of protein expression of PIEZO 1 and PIEZO 2 in adipocytes. D. Histogram of protein expression of Adiponectin and Perilipin in fibroblasts. E. Histogram of protein expression of Sca1 (right), Dlk (middle), and CD26 (right) in adipocytes. F. Bar graph quantifying adipocyte-fibroblast intra interactions. G. Bar graph quantifying Adipocyte 1-Fibroblast 4 interactions. H. Bar graph quantifying Adipocyte 2-Fibroblast 4 interactions.

FIG. 35: Comparison of cell-cell interactions in CODEX and Visium analysis at POD 14. A. Schematic to show comparison of spatial analysis at RNA and protein level. B. Differential interaction maps in PBS vs. P1i-treated wounds using CODEX analysis (left) and Visium analysis (right).

FIG. 36: VISIUM analysis at POD 14. A. Spatial plots of epithelial (Krt14), immune (Ptprc), fibroblast (Col1a1), and adipocyte (Adipoq) markers at POD 60 scars following PBS treatment. B. Spatial plots of mechanical markers (Piezo1, Piezo2, Yap1, and Ptk2) at POD 14 following PBS treatment (left) and P1i treatment (right).

FIG. 37: VISIUM analysis at POD 60, 105, and 150. Spatial plots of spatially variable features at POD 60 following PBS treatment (left) and P1i treatment (right).

FIG. 38: CytoTRACE analysis of scars at POD 60. Spatial plots of scars at POD 60 treated with P1i or PBS colored by CytoTRACE.

FIG. 39: CODEX analysis at POD 60, 105, and 150. A. Representative UMAPs of CD86 (left), PLIN1 (middle), and CD31 protein expression (right). B. Differential interaction maps of adipocytes and fibroblasts in PBS-treated wounds vs. P1i-treated wounds at POD 105. C. Differential interaction maps in PBS-treated wounds vs. P1i-treated wounds (left) at POD 60, POD 105 (middle), and POD 150 (right). D. Differential interaction maps in PBS-treated wounds (left) at POD 60, 105 and 150 and in P1i treated wounds (right) at POD 60, 105 and 150. E. Histogram of protein expression of PIEZO 1, PIEZO2, YAP, and FAK in adipocytes. F. Histogram of protein expression of COL1 in adipocytes. G. Bar graph quantifying Adipocyte-Fibroblast interactions.

FIG. 40: Analysis of human foreskin tissue xenografts. A. H&E (top) and Masson's trichrome (bottom) histology of ungrafted human foreskin samples. B. Left, UMAP of quantified extracellular matrix (ECM) ultrastructure parameters (each dot represents one histology image); right, picrosirius red histology of unwounded xenografted (UW) vs. native, ungrafted human foreskin. C. Quantitative reverse transcription PCR (RT-qPCR) quantification of Col1 expression by ungrafted versus unwounded grafted foreskin. D. Immunofluorescence (IF) staining for human-specific Col1 (hCol1, top) and mouse-specific Col 1 (mCol1, bottom; both, green signal) in consecutive sections of xenograft samples. White dotted lines indicate boundary between mouse skin (left side of each image) and xenografted human skin (right side). E. Top, RNAscope in situ hybridization of control (untreated) wounds (far left, far right) and P1i xenograft wounds (middle) targeting Piezo1 (left and middle panels) or positive control housekeeping gene (right panels; all, pink signal); black arrows indicate positively stained cells. Bottom, quantification of Piezo1⁺ cells per HPF in each wound condition from RNAscope.

Data shown as mean±S.D. *P≤0.05. Scale bars, 1 mm (A), 1 mm (B), 20 μm (D), 100 μm (E, first row), and 20 μm (E, second row).

FIG. 41: scRNA-seq analysis of human xenograft at POD 14. A. UMAP of all wound cells colored by cell type (left) and human/mouse origin (right). B. UMAP of all wound cells colored by wound treatment including unwounded, wounded, and P1i treated. C. Gene ontology pathways (GO) pathways of human cluster 0 (left) and human cluster 1 (right). D. Violin plots showing expression of indicated characteristic genes in cluster 0 (top) and cluster 1 (bottom). E. Expression of genes by pseudotime analysis with gene module 10 highlighted (indicated in black box) across pseudotime with selected genes including Thy1, Robo2, Trsp1, and Twist2. F. scVelo analysis of UMAP of all fibroblast cells. G. Expression of mechanical genes through pseudotime analysis coloured by Seurat cluster. H. Cell chat analysis of cell-cell interactions in wounded (red) and P1i-treated wounds (green) at POD 14. I. Heatmap of cell communications in P1i treated wounds versus untreated wounds at POD 14. in PBS and P1i treated wounds at POD 14 (black box showing interactions between human cluster 0 cells).

FIG. 42 Xenograft analysis following at POD 14. A. Immunostaining of xenograft wounds for CD29, Robo2, and CD90 in wounded and P1i-treated wounds at POD 14. B. Top: Schematic showing experimental plan. Bottom: Immunostaining of human fibroblasts sorted by cluster 3 markers for PPary and Collagen type 1 following stretching (top row), no stretch (middle row), and stretching with P1i inhibitor (P1i) (bottom row). C. Top: Schematic showing experimental plan. Bottom: Immunostaining of human fibroblasts for CD29, VCAM1, CJUN following stretching (top row), no stretch (second row), stretching with Wnt5a recombinant protein (third row), and stretching with P1i (bottom row). Data shown as mean±S.D. *P≤0.05. Scale bars, 100 μm (A), 200 μm (B).

FIG. 43: CODEX analysis of human xenografts. A. Representative UMAPs of Adiponectin (left), a-SMA (middle), and Piezo1 (right). B. Bar graphs showing cell proportion of adipocyte 3 and adipocyte 2 in PBS, P1i treated, and unwounded skin. C. Differential interaction maps in unwounded skin (top left), PBS treated skin (top right), and P1i treated skin (bottom). D. Differential interaction maps in unwounded vs. P1i-treated wounds (left) at POD 14. E. Bar graphs showing interactions of adipocyte and fibroblast cells in unwounded, PBS, and P1i treated wounds. F. Bar graphs showing interactions of adipocyte and other cell types in unwounded, PBS, and P1i treated wounds.

FIG. 44: CODEX analysis of human xenografts. A. Histogram of co-expression of either FAK⁺PIEZO1⁺PIEZO2⁺YAP⁺ (left), CD29⁺ROBO2⁺SFRP2⁺WNT5A⁺CALU⁺ (middle), and CJUN⁺PIEZO1⁺PIEZO2⁺ (right) in all cell types. B. Histogram of COL1⁺COL4⁺ in adipocyte clusters. C. Histogram of Ki67 in adipocyte clusters. D. Histogram of ADIPOQ⁺PLIN1⁺ in fibroblast clusters.

FIG. 45: Schematic illustrating the mechanism by which adipocytes convert to fibroblasts under mechanotransduction to cause scarring.

### DEFINITIONS

As used herein in its conventional sense, the term “fibroblast” refers to a cell responsible for synthesizing and organizing extracellular matrix. Two fibroblast lineages include Engrailed-1 lineage-negative fibroblasts (ENFs) and Engrailed-1 lineage-positive fibroblasts (EPFs). The EPF lineage includes all cells that express Engrailed-1 at any point during their development, and all progeny of those cells.

As used herein, the term “modulating” means increasing, reducing or inhibiting an attribute of a biological cell, population of cells, or a component of a cell (e.g., a protein, nucleic acid, etc.). In some cases, the attribute includes, e.g., activation of a signaling pathway. In some cases, the attribute includes an amount and/or activity of one or more cells. In some cases, the attribute includes, e.g., an amount, activity, or expression level (DNA or RNA expression levels) of a component of a cell (e.g., a protein, nucleic acid, etc.). In some cases, “modulate” or “modulating” or “modulation” may be measured using an appropriate in vitro assay, cellular assay or in vivo assay. In some cases, the increase or decrease is 10% or more relative to a reference, e.g., 10% or more, 20% or more, 30% or more, 40% or more, 50% or more, 60% or more, 70% or more, 80% or more, 90% or more, 95% or more, 97% or more, 98% or more, up to 100% relative to a reference. For example, the increase or decrease may be 2 or more times, 3 times or more, 4 times or more, 5 times or more, 6 times or more, 7 times or more, 8 times or more, 9 times or more, 10 times or more, 50 times or more, or 100 times or more relative to a reference.

The term “fibrosis” as used herein in its conventional sense refers to the formation or development of excess fibrous connective tissue in an organ or tissue as a result of injury or inflammation of a part or interference with its blood supply. It can be a consequence of the normal healing response that leads to a scar, an abnormal reactive process or no known or understood cause.

As used herein in its conventional sense, the term “scarring” refers to a condition in which fibrous tissue replaces normal tissue destroyed by injury or disease. The term “scarring” further refers to abnormality in one or more of color, contour (bulging/indentation), rugosity (roughness/smoothness), strength (skin strength is reduced/scars are weaker than skin), overall appearance, e.g., due to lack of hair (which does not regrow in scars), and texture (softness/hardness), arising during the skin healing process. The expression “preventing” or “prevent” used herein in the context of scarring refers to an adjustment to the extent of development of scarring, whereby one or more of the color, contour, rugosity and texture of the healed skin surface approximates on ordinary visual inspection to that of the subject's normal skin. The expression “reducing” or “reduce” used herein in the context of scarring refers to an adjustment to the extent of development of scarring, whereby one or more of the color, contour, rugosity and texture of the healed skin surface approaches measurably closer to that of the patient's normal skin.

As used herein in its conventional sense, the term “scar” refers to a fibrous tissue that replaces normal tissue destroyed by injury or disease. Damage to the outer layer of skin (the epidermis) is healed by rebuilding the tissue, and in these instances, scarring is slight or absent. When the thick layer of tissue beneath the skin's outer surface (i.e., the dermis) is damaged, however, rebuilding is more complicated. The body lays down collagen fibers (a protein which is naturally produced by the body) in a composition that is different from that found in uninjured skin, and this usually results in a noticeable scar. After the wound has healed, the scar continues to alter as new collagen is formed, existing collagen is enzymatically remodeled, and the blood vessels return to normal, allowing most scars to fade and improve in appearance over the two years following an injury. However, there permanently remains some visible evidence of the injury, and hair follicles and sweat and oil glands do not grow back. As used herein, the term “scar area” refers to the area of normal tissue that is destroyed by injury or disease and replaced by fibrous tissue.

Scars differ from normal skin in three key ways: (1) they are devoid of any dermal appendages (hair follicles, sweat glands, etc.); (2) their collagen structure is fundamentally different, with dense, parallel fibers rather than the “basketweave” pattern that lends normal skin its flexibility and strength; and (3) as a result of their inferior matrix structure, they are weaker than skin.

The term “scar-related gene” as used herein refers to a nucleic acid encoding a protein that is activated in response to scarring as part of the normal wound healing process. The term “scar-related gene product” as used herein refers to the protein that is expressed in response to scarring as part of the normal wound healing process.

Scar tissue consists mainly of disorganized collagenous extracellular matrix. This is produced by myofibroblasts, which differentiate from dermal fibroblasts in response to wounding, which causes a rise in the local concentration of Transforming Growth Factor-B, a secreted protein that exists in at least three isoforms called TGF-βI, TGF-β2 and TGF-β3 (referred to collectively as TGF-β). TGF-β is an important cytokine associated with fibrosis in many tissue types (Beanes, S. et al, Expert Reviews in Molecular Medicine, vol. 5, no. 8, pp. 1-22 (2003)). Types of scars are further described in, e.g., PCT Application No. WO 2014/040074, the disclosure of which is incorporated herein by reference in its entirety.

The term “skin” used herein in its conventional sense includes all surface tissues of the body and sub-surface structure thereat including, e.g., mucosal membranes and eye tissue as well as ordinary skin. The expression “skin” may include a wound zone itself. The re-approximation of skin over the surface of a wound has long been a primary sign of the completion of a significant portion of wound healing. This reclosure of the defect restores the protective function of the skin, which includes protection from bacteria, toxins, and mechanical forces, as well as providing the barrier to retain essential body fluids. The epidermis, which is composed of several layers beginning with the stratum corneum, is the outermost layer of the skin. The innermost skin layer is the deep dermis.

As used herein in its conventional sense, the term “dermal appendages” includes hair follicles, sebaceous and sweat glands, fingernails, and toenails.

As used herein, the term “dermal location” refers to a region of a skin of a subject having any size and area. The dermal location may encompass a portion of skin of a subject such as, e.g., the scalp. The dermal location may include one or more layers of skin including, e.g., the epidermis and the dermis. In some cases, the dermal location includes a wound.

As used herein in its conventional sense, a “photosensitizer” or “photoreactive agent” or “photosensitizing agent” is a light-activated drug or compound. A photosensitizer may be defined as a substance that absorbs electromagnetic radiation, most commonly in the visible spectrum, and releases it as another form of energy, most commonly as reactive oxygen species and/or as thermal energy. In some cases, a photosensitizing agent is useful in photodynamic therapy. Such agents may be capable of absorbing electromagnetic radiation and emitting energy sufficient to exert a therapeutic effect, e.g., the impairment or destruction of unwanted cells or tissue, or sufficient to be detected in diagnostic applications. For example, the photosensitizer can be any chemical compound that collects in one or more types of selected target tissues and, when exposed to light of a particular wavelength, absorbs the light and induces impairment or destruction of the target tissues. Virtually any chemical compound that homes to a selected target and absorbs light may be used. The photosensitizer may be nontoxic to a subject to which it is administered and is capable of being formulated in a nontoxic composition. The photosensitizer may also be nontoxic in its photodegraded form. In some cases, the photosensitizers are characterized by a lack of toxicity to cells in the absence of the photochemical effect and are readily cleared from non-target tissues.

As used herein in its conventional sense, the term “wound” includes any disruption and/or loss of normal tissue continuity in an internal or external body surface of a human or non-human animal body, e.g., resulting from a non-physiological process such as surgery or physical injury. The expression “wound” or “wound environment” used herein refers to any skin lesion capable of triggering a healing process which may potentially lead to scarring, and includes wounds created by injury, wounds created by burning, wounds created by disease and wounds created by surgical procedures. The wound may be present on any external or internal body surface and may be penetrating or non-penetrating. The methods herein described may be beneficial in treating problematic wounds on the skin's surface. Examples of wounds which may be treated in accordance with the method of the invention include both superficial and non-superficial wounds, e.g. abrasions, lacerations, wounds arising from thermal injuries (e.g. burns and those arising from any cryo-based treatment), and any wound resulting from surgery.

The term “wound healing” as used herein in its conventional sense refers to a regenerative process with the induction of a temporal and spatial healing program, including, but not limited to, the processes of inflammation, granulation, neovascularization, migration of fibroblast, endothelial and epithelial cells, extracellular matrix deposition, re-epithelialization, and remodeling.

The term “hair follicle formation” or “induction of hair follicle formation” as used herein in its conventional sense refers to a phenomenon in which dermal papilla cells induce epidermal cells to form the structure of the hair follicle.

The term “hair growth” or “induction of hair growth” as used herein in its conventional sense refers to a phenomenon in which hair matrix cells of the hair follicle differentiate and proliferate thereby forming the hair shaft, and dermal sheath cells act on the hair matrix or outer root sheath (ORS) to elongate the hair shaft from the body surface. In some cases, hair growth includes generating one or more new hair follicles. In some cases, hair growth includes generating one or more new hairs.

As used herein in its conventional sense, the term “alopecia” refers to a disease in which hair is lost. It can be due to a number of causes, such as androgenetic alopecia, trauma, radiotherapy, chemotherapy, iron deficiency or other nutritional deficiencies, autoimmune diseases and fungal infection. The loss of hair in alopecia is not limited just to head hair but can happen anywhere on the body. Alopecia is often accompanied by fading of hair color. Alopecia is often accompanied by deterioration of hair quality such as hair becoming finer or hair becoming shorter. With regard to types of alopecia, there are alopecia areata, androgenetic alopecia, postmenopausal alopecia, female pattern alopecia, seborrheic alopecia, alopecia pityroides, senile alopecia, cancer chemotherapy drug-induced alopecia, alopecia due to radiation exposure, trichotillomania, postpartum alopecia, etc. The types of alopecia are further described in U.S. Pat. No. 9,808,511, the entirety of which is incorporated by reference herein.

Alopecia areata is an auto-immune disease that can cause hair to fall out suddenly. Alopecia areata is alopecia in which coin-sized circular to patchy bald area(s) with a clear outline suddenly occur, without any subjective symptoms or prodromal symptoms, etc. in many cases, and subsequently when spontaneous recovery does not occur they gradually increase in area and become intractable. It may lead to bald patches on the scalp or other parts of the body. Hair growth in the affected hair follicles is reduced or completely ceases. Alopecia areata is known to be associated with an autoimmune disease such as a thyroid disease represented by Hashimoto's disease, vitiligo, systemic lupus erythematosus, rheumatoid arthritis, or myasthenia gravis or an atopic disease such as bronchial asthma, atopic dermatitis, or allergic rhinitis.

As used herein in its conventional sense, the term “microneedling” refers to the use of microneedles on an area of the body. An individual microneedle is designed to puncture the skin up to a predetermined distance, which may be greater than the nominal thickness of the stratum corneum layer of skin (the very outer layer of the skin out-covering the epidermis). Using such microneedles may overcome the barrier properties of the skin. At the same time, the microneedles are relatively painless and bloodless if they are made to not penetrate through the epidermis, which is approximately less than 2.0-2.5 mm beneath the outer surface of the skin. Microneedles may require a direct pushing motion against the skin of sufficient force to penetrate completely through the stratum corneum. In general, microneedle stimulation systems are well known for their use in skin care treatment of various conditions such as wrinkles, acne scarring, stretch marks, skin whitening and facial rejuvenation. In certain embodiments of microneedling, a method of piercing holes in the skin and applying drugs or cosmetics to the skin provides a way to rapidly and sufficiently permeate the skin. In some cases, using microneedles is sufficient to injure the skin just enough to begin natural healing processes and stimulate collagen and elastin production, and the like, to heal the skin. In these methods, hundreds to thousands of tiny holes or microconduits are created in the skin with the microneedling device without damaging the deeper layers of the skin. This injury to the skin begins a natural healing process that leads to the release of natural stimulants and growth factors which stimulates the formation of new natural collagen and elastin in the papillary dermis to produce new, healthy skin tissue. Also, new capillaries are formed. This neovascularisation and neocollagenesis associated with the wound healing process leads to the formation of younger looking skin, reduction of skin pathologies and improvement of scars. Generally called percutaneous collagen induction therapy, microneedling has also been used in the treatment of photo ageing. Furthermore, medical substances may be applied to the site where the holes are created and the substances are supposed to permeate into the skin through the tiny holes. Microneedling is generally applied to the face, neck, scalp, and just about anywhere on the body where a particular condition warrants without removing or permanently damaging the skin. A predetermined number of needles are inserted into the skin to the desired depth. As a reaction to the minor injury, the skin tissue begins a natural wound-healing cascade. This natural process forms new healthy dermal tissue that helps smooth scars, remove wrinkles and improve pigmentation, and yields a younger, healthier and a cleaner-looking skin.

As used herein in its conventional sense, the term “fractional laser resurfacing treatment” or “fractional laser resurfacing” or “fractional resurfacing” refers to the use of electromagnetic radiation to improve skin defects by inducing a thermal injury to the skin, which results in a complex wound healing response of the skin. This leads to a biological repair of the injured skin. Various techniques providing this objective have been introduced. The different techniques can be generally categorized in two groups of treatment modalities: ablative laser skin resurfacing (“LSR”) and non-ablative collagen remodeling (“NCR”). The first group of treatment modalities, i.e., LSR, includes causing thermal damage to the epidermis and/or dermis, while the second group, i.e., NCR, is designed to spare thermal damage of the epidermis. LSR with pulsed CO₂ or Er: YAG lasers, which may be referred to in the art as laser resurfacing or ablative resurfacing, is considered to be an effective treatment option for signs of photo aged skin, chronically aged skin, scars, superficial pigmented lesions, stretch marks, and superficial skin lesions. NCR techniques are variously referred to in the art as non-ablative resurfacing, non-ablative subsurfacing, or non-ablative skin remodeling. NCR techniques generally utilize non-ablative lasers, flashlamps, or radio frequency current to damage dermal tissue while sparing damage to the epidermal tissue. The concept behind NCR techniques is that the thermal damage of only the dermal tissues is thought to induce wound healing which results in a biological repair and a formation of new dermal collagen. This type of wound healing can result in a decrease of photoaging related structural damage. Avoiding epidermal damage in NCR techniques decreases the severity and duration of treatment related side effects. In particular, post procedural oozing, crusting, pigmentary changes and incidence of infections due to prolonged loss of the epidermal barrier function can usually be avoided by using the NCR techniques. Additional methods and devices for practicing fractional laser resurfacing are described in, e.g., PCT Application No. WO 2005/007003; U.S. application No. 20160324578; and Beasley et al. (2013) Current Dermatology Reports. 2:135-143, the disclosures of which are incorporated herein by reference in their entireties.

As used herein, the term “administering” includes in vivo administration as well as direct administration to tissues ex vivo. Generally, administration is, for example, oral, buccal, parenteral (e.g., intravenous, intraarterial, subcutaneous), intraperitoneal (i.e., into the body cavity), topically, e.g., by inhalation or aeration (i.e., through the mouth or nose), or rectally systemic (i.e., affecting the entire body). A composition may be administered in dosage unit formulations containing conventional non-toxic pharmaceutically acceptable carriers, adjuvants, and vehicles as desired. The term “topically” may include injection, insertion, implantation, topical application, or parenteral application.

### DETAILED DESCRIPTION

Methods of promoting healing of a wound in a dermal location of a subject are provided. Aspects of the methods may include administering an effective amount of a Piezo inhibitor composition to the wound to promote healing of the wound, e.g., by reducing transition of adipocytes to fibroblasts in the wound. Also provided are methods of both preventing new scarring during healing of a wound in a subject, and reversing/resolving existing scarring of a healed wound in a subject. Aspects of the methods may include forming a wound in a dermal location of a subject and administering an effective amount of a Piezo inhibitor composition to the wound (to promote regenerative healing of the wound) or to a healed wound (i.e., scar; to promote regenerative remodeling of the wound and resolution/reversal of the scar). Also provided are methods of preventing scarring during healing of a wound in a subject. Aspects of the methods may include forming a wound in a dermal location of a subject and administering an effective amount of a Piezo inhibitor composition to the wound to promote healing of the wound. Also provided are methods of ameliorating, e.g., reducing or inhibiting, organ fibrosis, e.g., liver fibrosis, heart fibrosis, inflammatory bowel fibrosis and muscle fibrosis in a subject by administering to the subject an effective amount of a Piezo inhibitor composition. Also provided are kits including an amount of a Piezo inhibitor composition.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, the methods are reviewed first in greater detail, followed by a review of kits. Applications in which the methods and kits find use are also provided in greater detail below.

### Methods

As summarized above, aspects of the methods include methods of promoting healing of a wound in a dermal location of a subject. In some cases, the methods prevent scarring during healing of a wound in a subject. In some cases, the methods promote hair growth on a subject. In certain embodiments, aspects of the methods include administering an effective amount of a Piezo inhibitor to a wound to promote healing of the wound. The methods may be applied to any cell or population of cells as described herein.

In some cases, the methods include modulating mechanical signaling through a mechanical signaling pathway or mechano-transduction pathway in one or more cells, e.g., in a wound environment. The one or more cells may adipocytes. As used herein, the term “mechanical activation” refers to activation of a mechanical signaling pathway in one or more adipocytes in response to mechanical cues within a wound environment. The mechanical cues can include, e.g., mechanical tension, extracellular matrix (ECM) rigidity, strain, shear stress, or adhesive area. In some cases, activation of the mechanical signaling pathway in the one or more adipocytes contributes to fibrosis and scarring after wounding. In some cases, the mechanical signaling pathway converts mechanical cues, e.g., in a wound environment, into transcriptional changes such as, e.g., expression of pro-fibrotic genes in the one or more adipocytes. The mechanical signaling pathway may include Piezo protein, e.g., Piezo-Type Mechanosensitive Ion Channel Component 1 (i.e., Piezo1, (UniProtKB-Q92508 (PIEZ1_HUMAN)) and/or Piezo-Type Mechanosensitive Ion Channel Component 2 (i.e., Piezo2 (UniProtKB-Q9H515 (PIEZ2_HUMAN)) as the molecular effector, e.g., that mediates transition of adipocytes to fibroblasts. For example, Piezo1 inhibition through GsMTx4 blocks TRPC1 and TRPC6. (Copp S W, Kim J S, Ruiz-Velasco V, Kaufman M P. The mechano-gated channel inhibitor GsMTx4 reduces the exercise pressor reflex in rats with ligated femoral arteries. Am J Physiol Heart Circ Physiol. 2016; 310 (9): H1233-41). Piezo1 inhibition GsMTx4 may also inhibit TRPC1 and TRPC6 channels to block the transition from an adipocyte to fibroblast. Piezo2 inhibition via D-Gs MTx4 may also inhibit piezo 1/2 proteins an contribute to the blockade of the transition from an adipocyte to fibroblast (Alcaino C, Knutson K, Gottlieb P A, Farrugia G, Beyder A. Mechanosensitive ion channel Piezo2 is inhibited by D-GsMTx4. Channels (Austin). 2017 May 4; 11 (3): 245-253. doi: 10.1080/19336950.2017.1279370).

As summarized above, aspects of the methods may include administering an effective amount of a Piezo inhibitor to a wound. The administration may promote healing of a wound. In some cases, the administration modulates mechanical activation of one or more cells, e.g., adipocytes, in the wound. In certain embodiments, the Piezo inhibitor includes a Piezo1 and/or Piezo2 inhibitor. In some cases, the Piezo inhibitor is a Piezo1 inhibitor. In some cases, the Piezo inhibitor is a Piezo2 inhibitor. In some cases, both a Piezo1 inhibitor and Piezo2 inhibitor are administered to a subject. In some cases, the method consists essentially of administering a Piezo inhibitor. As used herein, a “Piezo inhibitor” refers to a molecule that may inhibit Piezo protein function and signaling. In some cases, the Piezo inhibitor inhibits cellular mechanical signaling. In some cases, the Piezo inhibitor reduces or inhibits Piezo protein expression (DNA or RNA expression) or activity (e.g., nuclear translocation). In some cases, the Piezo inhibitor reduces or inhibits the interaction of a Piezo protein with other signaling molecules. In certain embodiments, administering the Piezo inhibitor reduces mechanical activation of one or more cells, e.g., adipocytes, in a wound, wherein, e.g., the level of mechanical activation of the one or more cells, e.g., adipocytes, in a wound is reduced compared to a suitable control.

As used herein, “an effective amount of a Piezo inhibitor” refers to an amount of a Piezo inhibitor suitable to promote healing of a wound. In some cases, an effective amount of a Piezo inhibitor includes one or more unit doses of the Piezo inhibitor such as, e.g., one or more doses, two or more doses, three or more doses, four or more doses, five or more doses, six or more doses, seven or more doses, eight or more doses, nine or more doses, or ten or more doses. In some cases, an effective amount of a Piezo inhibitor composition includes a single dose, e.g., a single injection, of the Piezo inhibitor. The Piezo inhibitor may include any suitable amount of Piezo inhibitor such as, e.g., an effective amount of a Piezo inhibitor. In some cases, the effective amount of a Piezo inhibitor does not delay wound closure or the wound closure rate. In some cases, the Piezo inhibitor c includes an effective amount of a Piezo inhibitor ranging from, e.g., 0.1 mg/ml to 2 mg/ml, 0.5 mg/ml to 2 mg/ml, 1 mg/ml to 2 mg/ml, 0.1 mg/ml to 1 mg/ml, 0.5 mg/ml to 1 mg/ml, or 1 mg/ml to 5 mg/ml. The effective amount of the Piezo inhibitor may be administered, e.g., after wound formation, over any suitable period of time including, e.g., one day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more.

In some instances, the Piezo inhibitor is a small molecule agent that exhibits the desired activity, e.g., inhibiting Piezo mediated transition of adipocytes to fibroblasts. Naturally occurring or synthetic small molecule compounds of interest include numerous chemical classes, such as organic molecules, e.g., small organic compounds having a molecular weight of more than 50 and less than about 2,500 Daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols.

In some cases, the Piezo inhibitor is a protein or fragment thereof or a protein complex. In some cases, the Piezo inhibitor is an antibody binding agent or derivative thereof. The term “antibody binding agent” as used herein includes polyclonal or monoclonal antibodies or fragments that are sufficient to bind to an analyte of interest, e.g., Piezo. The antibody fragments can be, for example, monomeric Fab fragments, monomeric Fab′ fragments, or dimeric F (ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. In some cases, the Piezo inhibitor is an enzyme or enzyme complex. In some cases, the Piezo inhibitor includes a phosphorylating enzyme, e.g., a kinase. In some cases, the Piezo inhibitor is a complex including a guide RNA and a CRISPR effector protein, e.g., Cas9, used for targeted cleavage of a nucleic acid.

In some cases, the Piezo inhibitor is a nucleic acid. The nucleic acids may include DNA or RNA molecules. In certain embodiments, the nucleic acids modulate, e.g., inhibit or reduce, the activity of a gene or protein, e.g., by reducing or downregulating the expression of the gene. The nucleic acid may be a single stranded or double-stranded and may include modified or unmodified nucleotides or non-nucleotides or various mixtures and combinations thereof. In some cases, the Piezo inhibitor includes intracellular gene silencing molecules by way of RNA splicing and molecules that provide an antisense oligonucleotide effect or an RNA interference (RNAi) effect useful for inhibiting gene function. In some cases, gene silencing molecules, such as, e.g., antisense RNA, short temporary RNA (stRNA), double-stranded RNA (dsRNA), small interfering RNA (siRNA), short hairpin RNA (shRNA), microRNA (miRNA), tiny non-coding RNA (tncRNA), snRNA, snoRNA, and other RNAi-like small RNA constructs, may be used to target a protein-coding as well as non-protein-coding genes. In some case, the nucleic acids include aptamers (e.g., spiegelmers). In some cases, the nucleic acids include antisense compounds. In some cases, the nucleic acids include molecules which may be utilized in RNA interference (RNAi) such as double stranded RNA including small interfering RNA (siRNA), locked nucleic acid (LNA) inhibitors, peptide nucleic acid (PNA) inhibitors, etc.

Piezo inhibitors finding use in embodiments of the invention include, but are not limited to: Piezo1/2 inhibitor GsMTx4 (Qin, L., He, T., Chen, S. et al. Roles of mechanosensitive channel Piezo1/2 proteins in skeleton and other tissues. Bone Res 9, 44 (2021)) and the like.

In some embodiments, the Piezo inhibitor is administered as a pharmaceutically acceptable composition in which one or more Piezo inhibitors may be mixed with one or more carriers, thickeners, diluents, buffers, preservatives, surface active agents, excipients and the like. Pharmaceutical compositions may also include one or more additional active ingredients such as antimicrobial agents, anti-inflammatory agents, anesthetics, and the like in addition to the one or more Piezo inhibitors. In some cases, the Piezo inhibitor composition includes, e.g., a derivative of Piezo inhibitor. “Derivatives” include pharmaceutically acceptable salts and chemically modified agents.

The pharmaceutical compositions of the present invention may be administered by any route commonly used to administer pharmaceutical compositions. For example, administration may be done topically (including ophthalmically, vaginally, rectally, intranasally), orally, by inhalation, or parenterally, for example by intravenous drip or subcutaneous, intraperitoneal or intramuscular injection.

Pharmaceutical compositions formulated for topical administration may include ointments, lotions, creams, gels, drops, sprays, liquids, salves, sticks, soaps, aerosols, and powders. Any conventional pharmaceutical excipient, such as carriers, aqueous, powder or oily bases, thickeners and the like may be used. Ointments and creams may, for example, be formulated with an aqueous or oily base with the addition of suitable thickening and/or gelling agents. Lotions may be formulated with an aqueous or oily base and will, in general, also contain one or more emulsifying, dispersing, suspending, thickening or coloring agents. Powders may be formed with the aid of any suitable powder base. Drops may be formulated with an aqueous or non-aqueous base also comprising one or more dispersing, solubilizing or suspending agents. Aerosol sprays are conveniently delivered from pressurized packs, with the use of a suitable propellant.

The Piezo inhibitor composition may be stored at any suitable temperature. In some cases, the Piezo inhibitor composition is stored at temperatures ranging from 1° C. to 30° C., from 2° C. to 27° C., or from 5° C. to 25° C. The Piezo inhibitor composition may be stored in any suitable container, as described in detail below.

The Piezo inhibitor composition may be administered to a wound in a dermal location a subject. In some cases, the Piezo inhibitor composition is administered to a dermal location surrounding a wound in a subject. The administration can be by any suitable route, including, e.g., topical, intravenous, intradermal, subcutaneous, and intramuscular. In some cases, the administering comprises injecting the composition below a topical dermal location of the subject. The injecting may be performed with any suitable device such as, e.g., a needle. Other delivery means include coated microneedles, i.e. microneedles having a Piezo inhibitor composition deposited thereon, as well as microneedles that include internal reservoirs that are configured to receive a Piezo inhibitor composition therein and disperse the Piezo inhibitor composition therefrom. In some cases, the administering comprises delivering the composition to a topical dermal location. The delivering may be performed with any suitable device or composition such as, e.g., a transdermal patches, gels, creams, ointments, sprays, lotions, salves, sticks, soaps, powders, pessaries, aerosols, drops, solutions and any other convenient pharmaceutical forms.

The Piezo inhibitor composition may be administered at any suitable time. In some cases, the Piezo inhibitor composition is administered to a wound immediately after formation of the wound in a subject. In some cases, the Piezo inhibitor composition is administered to a wound after any suitable amount of time after formation of the wound such as, e.g., 1 minute, 2 minutes, 5 minutes, 10 minutes, 30 minutes, or an hour after formation of the wound. In some cases, the Piezo inhibitor composition is administered to a closed wound after any suitable amount of time after formation of the wound such as, e.g., 7 days or longer, 14 days or longer, 30 days or longer, 60 days or longer, 120 days or longer, 150 days or longer, 180 days or longer, 210 days or longer, 240 days or longer, 270 days or longer, 300 days or longer, 330 days or longer, 360 days or longer, 1 year or longer, 1.5 years or longer, 2 years or longer, 5 years or longer, etc.

In certain embodiments, the methods as provided herein promote healing of a wound. In certain embodiments, the methods as provided herein promote adipocyte-mediated healing of a wound. As used herein, the term “adipocyte-mediated healing” refers to healing of a wound associated with the presence and/or activity of adipocytes in the wound. In some cases, the healing, e.g., adipocyte-mediated healing, includes a regenerative response from one or more cells. In some cases, the methods do not compromise healing of a wound, e.g., wound closure and repair. For example, in some cases, the methods do not delay wound closure or the wound closure rate. In some cases, the healing, e.g., adipocyte-mediated healing, of the wound is completed in an amount of time substantially equal to an amount of time for healing of a wound not treated with the Piezo inhibitor composition. In some cases, the healing, e.g., adipocyte-mediated healing, of the wound is completed in an amount of time that is less than an amount of time for healing of a wound not treated with the adipocyte inhibitor composition, i.e., the healing, e.g., adipocyte-mediated healing, of the wound is accelerated compared to the healing of a wound not treated with the adipocyte inhibitor composition. In certain embodiments, the methods reduce, prevent, or reverse scarring during healing of a wound in a subject, as described in detail below.

In some cases, the healing, e.g., adipocyte-mediated healing, of the wound includes regeneration of dermal appendages. In some cases, the dermal appendages include hair follicles, sweat glands, and sebaceous glands. In certain embodiments, the methods provided herein promote hair growth on a subject, as described in detail below. In certain embodiments, the methods provided herein treat a subject for alopecia, e.g., by promoting hair growth in areas of hair loss, as described in detail below. In some cases, the healing, e.g., adipocyte-mediated healing, of the wound produces a healed wound with reduced levels of collagen hyperproliferation compared to levels of collagen hyperproliferation in a healed wound not treated with the Piezo inhibitor composition. In some cases, the healing, e.g., adipocyte-mediated healing, of the wound produces a healed wound comprising improved connective tissue architecture compared to the connective tissue architecture in a healed wound not treated with the Piezo inhibitor composition. In certain embodiments, the healing, e.g., adipocyte-mediated healing, includes recovery or regrowth of one or more of dermal appendages, ultrastructure (i.e., matrix structure), and mechanical strength (e.g., wound breaking strength) that is, e.g., comparable to that of normal skin or unwounded skin.

In certain embodiments, the methods further include forming a wound in a dermal location of a subject. In some cases, the wound is formed to perform a procedure, e.g., a surgical procedure. In some cases, the wound is formed to improve tissue quality. For example, the methods may include forming microscopic injuries to induce tissue regeneration. In some cases, the wound is formed to disrupt an outer dermal layer, e.g., stratum corneum, to increase penetration and absorption of one or more substances or compositions, e.g., a therapeutic composition, through the skin of a subject. In some cases, the methods include forming one or more wounds at a plurality of dermal locations. In some cases, the methods include forming one or more wounds across a dermal location. The nature and size of the wound may vary. In certain embodiments, the wound is a microscopic wound. The microscopic wound may be formed by any suitable means as described in detail below such as, e.g., a laser, microneedle, etc. In certain embodiments, the wound is a partially healed wound.

The wound may be formed by any suitable means, e.g., mechanical, physical or chemical injury of the skin. In some cases, the wound results from non-physiological processes, e.g., a surgical wound or a wound resulting from physical injury, abrasions, lacerations, thermal injuries (e.g., a burn or a wound arising from a cryo-based treatment). In some cases, the wound is formed by the application of one or more of, e.g., ultrasound, radio frequency (RF), laser (e.g., fraxel), ultraviolet energy, infrared energy, or mechanical disruption. In some cases, the wound is formed by, e.g., microdermabrasion (e.g., with an adapted skin preparation pad, sandpaper), microneedling, tape-stripping, pan-scrubber, exfoliating scrub, compress rubbing, non-ablative lasers at a low-energy delivery. Additional mechanical treatments include, e.g., curettage or dermoabrasion (e.g., with an adapted sandpaper or micro-needling (or micro-perforation)). In certain aspects, wounding is accomplished using chemical treatments (e.g., a caustic agent, etc.), or mechanical or electromagnetic or physical treatments including but not limited to dermabrasion (DA), particle-mediated dermabrasion (PMDA), microdermabrasion, microneedles, laser (e.g., a laser that delivers ablative, non-ablative, fractional, non-fractional, superficial, or deep treatment, and/or that is CO₂-based, or erbium-YAG-based, erbium-glass based (e.g. Sciton Laser), neodymium: yttrium aluminum garnet (Nd: YAG) laser, etc.), a low-level (low-intensity) laser therapy treatment (e.g., HairMax® Laser comb), laser abrasion, irradiation, radio frequency (RF) ablation, dermatome planing (e.g. dermaplaning), a coring needle, a puncture device, a punch tool or other surgical tool, suction tool or instrument, electrology, electromagnetic disruption, electroporation, sonoporation, low voltage electric current, intense pulsed light, or surgical treatments (e.g., skin graft, hair transplantation, strip harvesting, scalp reduction, hair transplant, follicular unit extraction (FUE), robotic FUE, etc.), or supersonically accelerated saline. In some cases, the wound is formed by a tissue disrupting device, as described in detail below.

Embodiments of the methods of the present invention can be practiced on any suitable subject. A subject of the present invention may be a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

### Scar Reduction

In certain embodiments, the methods provided herein reduce, reverse, or prevent scarring during healing of a wound in a subject. In certain embodiments, the methods include forming a wound in a dermal location of a subject, e.g., according to any of the embodiments described herein, and administering an effective amount of a Piezo inhibitor composition to the wound to promote healing of the wound, e.g., according to any of the embodiments described herein. In certain embodiments, the methods include forming a wound in a dermal location of a subject, e.g., according to any of the embodiments described herein, and administering an effective amount of a Piezo inhibitor composition to the wound to modulate adipocyte to fibroblast transition in the wound to promote healing of the wound, e.g., according to any of the embodiments described herein.

The level or amount of scarring may be assessed and measured according to any convenient metric. The levels of scarring, e.g., in a wound treated with a Piezo inhibitor composition during healing or a healed wound treated with a Piezo inhibitor composition, may be assessed relative to a control, e.g., a wound or healed wound not treated with a Piezo inhibitor composition. In some cases, the level of scarring is assessed by measuring a physical property of a healed wound such as, e.g., tensile strength, scar area, etc. In some cases, the level of scarring is assessed by detecting the presence of or quantitating the amount of one or more dermal appendages including, e.g., hair follicles, sweat glands, and sebaceous glands, in a dermal location. In some cases, the level of scarring is assessed by detecting and/or characterizing the formation of connective tissue or an ECM matrix in a dermal location. In certain embodiments, the level of scarring is assessed by detecting and/or quantitating the amount of cells, e.g., types or subpopulations of cells, in a dermal location. In some cases, the level of scarring is assessed by measuring and/or quantitating the expression and/or activity or one or more scar-related genes and/or scar-related gene products. In some cases, levels of scarring are assessed by one or more of the following: visual examination, histology, immunohistochemical analysis, immunofluorescence, and machine learning. In some cases, the level of scarring is assessed with a machine learning algorithm for quantitatively assessing connective tissue and fibrosis based on histological stains. In some embodiments, evaluated metrics include, e.g., ECM fiber length and width, packing and alignment of groups of ECM fibers, and ECM fiber branching. Various scar assessment scales are provided, e.g., in PCT Application No. WO 2014/040074, the disclosure of which is incorporated herein by reference in its entirety. According to some embodiments, the methods reduce scarring compared to a control as measured by visual analog scale (VAS) score, color matching (CM), matte/shiny (M/S) assessment, contour (C) assessment, distortion (D) assessment, texture (T) assessment, or a combination thereof. While the magnitude of scarring reduction may vary, in some instances the magnitude ranges from 10% to 98%, such as, 10% to 95%, 20% to 95%, 30% to 95%, 40% to 95%, 50% to 95%, 60% to 95%, 70% to 95%, 80% to 95%, or 90% to 95%.

The levels of reduction of scarring during the healing process may vary. In certain embodiments, the methods are effective to reduce the occurrence, severity, or both of scars. In some cases, the method produces a healed wound with reduced levels of scarring compared to levels of scarring in a healed wound not treated with the Piezo inhibitor composition. In certain embodiments, the method produces a scar-less healed wound. In some cases, the methods produce a healed wound comprising improved connective tissue architecture compared to the connective tissue architecture in a healed wound not treated with the Piezo inhibitor composition. In some cases, the methods produce a healed wound with reduced levels of collagen hyperproliferation compared to levels of collagen hyperproliferation in a healed wound not treated with the Piezo inhibitor composition. In some embodiments, the methods improve the alignment of collagen fibers in the wound. In some embodiments, the methods reduce collagen formation in the wound. In some cases, the methods produce a healed wound with increased growth of dermal appendages. In certain embodiments, the methods reduce the wound size. In some case, a dermal location having a healed wound treated with a Piezo inhibitor composition according to the methods provided herein is indistinguishable in appearance (e.g., pigmentation, texture) from normal skin or unwounded skin. In some cases, a dermal location having a healed wound treated with a Piezo inhibitor composition according to the methods provided herein has physical properties (e.g., tensile strength) indistinguishable from normal skin or unwounded skin. In some cases, a dermal location having a healed wound treated with a Piezo inhibitor composition according to the methods provided herein has growth and generation of dermal appendages that are indistinguishable from normal skin or unwounded skin. In some cases, a dermal location having a healed wound treated with a Piezo inhibitor composition according to the methods provided herein has a connective tissue architecture, e.g., ECM matrix, that is indistinguishable from normal skin or unwounded skin. In certain embodiments, the methods do not impair normal wound healing or delay the wound closure rate compared to a control. In certain embodiments, the methods increase wound healing, e.g., the wound closure rate compared to a control. In some cases, one or more of the produced effects of the methods as described herein indicate a reduction of scarring or the prevention of scarring.

According to some embodiments, the methods decrease scar area compared to a control. According to some embodiments, the methods decrease scar area compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods decrease scar area compared to a control within one day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a Piezo inhibitor composition.

According to some embodiments, the methods decrease fibrosis at a dermal location compared to a control. In some cases, the methods decrease fibrosis at a dermal location compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods decrease fibrosis at a dermal location compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration.

According to some embodiments, the methods produce a wound or healed wound with increased tensile strength, e.g., as measured by wound breaking force and Young's modulus, compared to a control. According to some embodiments, the methods increase tensile strength compared to a control within one day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration. According to some embodiments, the methods increase tensile strength compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more.

According to some embodiments, the methods produce detectible levels of dermal appendages such as hair follicles, sweat glands, and/or sebaceous glands, or any combination thereof, at a dermal location compared to a control. According to some embodiments, the methods increase the number of dermal appendages such as hair follicles, sweat glands, and/or sebaceous glands, or any combination thereof, at a dermal location compared to a control. In some cases, the methods increase the number of dermal appendages such as hair follicles, sweat glands, and/or sebaceous glands, or any combination thereof, at a dermal location compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods produce detectible levels of or increase the number of dermal appendages such as hair follicles, sweat glands, and/or sebaceous glands, or any combination thereof, at a dermal location compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration.

According to some embodiments, the methods increase the number of hairs at a dermal location compared to a control. In some cases, the methods increase the number of hairs at a dermal location compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods increase the number of hairs at a dermal location compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration.

In some embodiments, the methods may modulate the expression and/or activity of scar-related genes or the production of scar-related gene products. In some cases, the level of scarring may be assessed by measuring the expression and/or activity of scar-related genes. In some cases, the level of scarring may be assessed by measuring the amount and/or activity of scar-related gene products. According to another embodiment, an effective amount of a Piezo inhibitor composition is effective to modulate messenger RNA (mRNA) levels expressed from scar-related genes. According to another embodiment, an effective amount of a Piezo inhibitor composition is effective to modulate the level of scar-related gene product expressed from the scar related gene. According to some embodiments, the scar-related gene and/or product is transforming growth factor-β1 (TGF-31), tumor necrosis factor-α (TNF-α), collagen, interleukin-6 (IL-6), chemokine (CC motif) Ligand 2 (CCL2) (or monocyte chemotactic protein-1 (MCP-1)), chemokine (CC motif) receptor 2 (CCR2), EGF-like module-containing mucin-like hormone receptor-like 1 (EMR1), CD26, YAP, fibronectin, or one or more of the sma/mad-related proteins (SMAD). According to some embodiments, the methods modulate, e.g., decrease, the expression and/activity of one or more of collagen type 1, CD26, and YAP in a wound, e.g., in cells present in a wound, compared to a control. According to some embodiments, the methods modulate, e.g., increase, the expression and/activity of fibronectin in a wound, e.g., in cells present in a wound, compared to a control. According to some embodiments, the methods produce detectible levels of markers of hair follicle and sebaceous or sweat gland identity such as, e.g., cytokeratin 14 and/or cytokeratin 19, respectively, at a dermal location compared to a control. In some cases, the methods increase the levels of markers of hair follicle and sebaceous or sweat gland identity, e.g., cytokeratin 14 and/or cytokeratin 19, at a dermal location compared to a control.

In certain embodiments, the methods decrease or increase the expression and/activity of one or more scar-related genes or scar-related gene products by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods decrease or increase the expression and/or activity of one or more scar-related genes or scar-related gene products compared to a control within one day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a Piezo inhibitor composition.

In some instances, the methods reverse existing scarring in healed wounds/existing scars. In such embodiments, one or more metrics of scars, e.g., as described above, may be improved as described above. The existing scar may be scar that is has existed for a period of time, e.g., 7 days or longer, 14 days or longer, 30 days or longer, 60 days or longer, 120 days or longer, 150 days or longer, 180 days or longer, 210 days or longer, 240 days or longer, 270 days or longer, 300 days or longer, 330 days or longer, 360 days or longer, 1 year or longer, 1.5 years or longer, 2 years or longer, 5 years or longer, etc.

### Hair Growth

In certain embodiments, the methods provided herein promote hair growth on a subject in a dermal location. In some embodiments, the subject may have alopecia and/or have been diagnosed with alopecia. In certain embodiments, the methods are methods for treating a subject for alopecia, e.g., by promoting hair growth in a dermal location of hair loss. In certain embodiments, the methods include forming a wound in a dermal location of a subject where hair growth is desired, e.g., according to any of the embodiments described herein, and administering an effective amount of a Piezo inhibitor composition to the wound to promote healing of the wound, e.g., according to any of the embodiments described herein. In certain embodiments, the methods may include forming a wound in a dermal location where hair growth is desired of a subject, e.g., according to any of the embodiments described herein, and administering an effective amount of a Piezo inhibitor composition to the wound.

In certain embodiments, the methods provided herein promote hair growth on a subject. The methods may induce or promote hair growth at any suitable dermal location in a subject. In certain embodiments, the methods promote or induce hair growth in a dermal location devoid of dermal appendages, e.g., hair follicles, sweat glands, etc. In some cases, the dermal location is hairless. In some cases, the dermal location includes a scar. In certain embodiments, the methods promote or induce hair growth in a dermal location having dermal appendages. In some cases, the dermal location includes hair. The dermal location may be located at any portion of the body where hair may naturally grow such as, e.g., the scalp, face, legs, arms, etc. In certain embodiments, the dermal location is present on a hairless area of the scalp of a subject. In certain embodiments, the dermal location includes the entire surface of the scalp of a subject.

The level of hair growth may be assessed and measured according to any convenient metric. The levels of hair growth may be assessed relative to a control, e.g., a dermal location characterized by hair loss, a dermal location devoid of dermal appendages, a wound not treated with a Piezo inhibitor composition, or healed wound not treated with a Piezo inhibitor composition. In certain embodiments, hair growth is determined by detecting the presence of new hairs appearing in a dermal location. In this method, hair growth may be confirmed when tips of the new hairs appear on the treatment area. Hair growth may also be determined by detecting hair follicle formation and/or measuring an increase in length of the hair follicles. In some cases, hair growth includes generating one or more new hair follicles. Hair growth may also be determined by measuring a change in the hairline. In some cases, the change in the hairline is determined by measuring the change in distance between any point on the hairline and the browline of the subject's head. In some cases, the methods decrease the amount of hair falling out compared to a control. In some cases, the methods prevent the progress of hair loss. In certain embodiments, there is no recurrence of hair loss permanently or for a period of time after performing the methods including, e.g., one month or more, two months or more, three months or more, half a year or more, one year or more, two years or more, three year or more, five years or more, or ten years or more.

According to some embodiments, the methods decrease the amount of hair loss compared to a control. In some cases, the methods decrease the amount of hair loss compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods decrease the amount of hair loss compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a Piezo inhibitor composition.

According to some embodiments, the methods increase the number of hair follicles at a dermal location, e.g., treated with a Piezo inhibitor composition, compared to a control. In some cases, the methods increase the number of hair follicles at a dermal location compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods increase the number of hair follicles at a dermal location compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a Piezo inhibitor composition.

According to some embodiments, the methods increase the number of hairs at a dermal location compared to a control. In some cases, the methods increase the number of hairs at a dermal location compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods increase the number of hairs at a dermal location compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a Piezo inhibitor composition.

### Organ Fibrosis

While embodiments of the invention are described above primarily in terms of dermal conditions, e.g., skin scarring, the invention is not so limited. Embodiments of the invention target fibrosis of organs other than skin. As such, also provided are methods of ameliorating, e.g., reducing or inhibiting, organ fibrosis, e.g., liver fibrosis, heart fibrosis, inflammatory bowel fibrosis, muscle fibrosis, kidney fibrosis, etc., in a subject by administering to the subject an effective amount of a Piezo inhibitor composition to treat the organ fibrosis. In some instances, methods of treating a subject for liver fibrosis (Basaranoglu M, Basaranoglu G, Sentürk H. From fatty liver to fibrosis: a tale of “second hit”. World J Gastroenterol. 2013; 19 (8): 1158-1165) are provided, where such methods include administering to the subject an effective amount of a Piezo inhibitor composition. In some instances, methods of treating a subject for heart fibrosis (Ng A C T, Strudwick M, van der Geest R J, Ng A C C, Gillinder L, Goo S Y, Cowin G, Delgado V, Wang W Y S, Bax J J. Impact of Epicardial Adipose Tissue, Left Ventricular Myocardial Fat Content, and Interstitial Fibrosis on Myocardial Contractile Function. Circ Cardiovasc Imaging. 2018 August; 11 (8): e007372.) are provided, where such methods include administering to the subject an effective amount of a Piezo inhibitor composition. In some instances, methods of treating a subject for inflammatory bowel fibrosis (Mao R, Kurada S, Gordon I O, Baker M E, Gandhi N, McDonald C, Coffey J C, Rieder F. The Mesenteric Fat and Intestinal Muscle Interface: Creeping Fat Influencing Stricture Formation in Crohn's Disease. Inflamm Bowel Dis. 2019 Feb. 21; 25 (3): 421-426) are provided, where such methods include administering to the subject an effective amount of a Piezo inhibitor composition. In some instances, methods of treating a subject for muscle fibrosis (Klingler W, Jurkat-Rott K, Lehmann-Horn F, Schleip R. The role of fibrosis in Duchenne muscular dystrophy. Acta Myol. 2012; 31 (3): 184-195.) are provided, where such methods include administering to the subject an effective amount of a Piezo inhibitor composition. In some instances, methods of treating a subject for kidney fibrosis (Gai Z, Wang T, Visentin M, Kullak-Ublick G A, Fu X, Wang Z. Lipid Accumulation and Chronic Kidney Disease. Nutrients. 2019; 11 (4): 722. Published 2019 Mar. 28. doi: 10.3390/nu11040722) are provided, where such methods include administering to the subject an effective amount of a Piezo inhibitor composition.

### Combination Therapy

For use in the subject methods, the Piezo inhibitor(s), such as described above, may be administered in combination with other pharmaceutically active agents, including other agents that treat the underlying condition or a symptom of the condition, e.g., scarring. “In combination with” as used herein refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

Examples of other agents for use in combination therapy in embodiments of methods of the invention include, but are not limited to, YAP inhibitors. In some instances, the YAP inhibitor is a small molecule agent that exhibits the desired activity, e.g., inhibiting YAP expression and/or activity. Naturally occurring or synthetic small molecule compounds of interest include numerous chemical classes, such as organic molecules, e.g., small organic compounds having a molecular weight of more than 50 and less than about 2,500 Daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols.

In some cases, the YAP inhibitor is a photosensitizing agent. In some cases, the Yap inhibitor is a benzoporphyrin derivative (BPD). The benzoporphyrin derivative may be any convenient benzoporphyrin derivative such as, e.g., those described in U.S. Pat. Nos. 5,880,145; 6,878,253; 10,272,261; and U.S. Application No. 2009/0304803, the disclosures of which are incorporated herein by reference in their entireties. In some cases, the benzoporphyrin derivative is a photosensitizing agent. In some cases, the YAP inhibitor is verteporfin (benzoporphyrin derivative monoacid ring A, BPD-MA; tradename: Visudyne®).

Further details regarding YAP inhibitors and methods of using the same are provided in U.S. patent application Ser. No. 17/626,699; the disclosure of which is herein incorporated by reference.

In the context of a combination therapy, combination therapy compounds may be administered by the same route of administration (e.g., intrapulmonary, oral, enteral, etc.) that the mutant Listeria are administered. In the alternative, the compounds for use in combination therapy with the mutant Listeria may be administered by a different route of administration.

### Kits

Aspects of the present disclosure also include kits. The kits are suitable for practicing embodiments of the methods described herein. The kits may include, e.g., an amount of a Piezo inhibitor composition. In some instances, the kits may further include a tissue disrupting device. In some cases, the kits are suitable for practicing embodiments of the methods for promoting wound healing. In some cases, the kits are suitable for practicing embodiments of the methods for promoting hair growth. In some cases, the kits are suitable for practicing embodiments of the methods for ameliorating scar formation. In some cases, the kits are suitable for practicing embodiments of the methods for treating a subject for alopecia.

The Piezo inhibitor composition may be present in any suitable amount. In some cases, the kit includes an effective amount of a Piezo inhibitor composition, e.g., according to the embodiments described above. The Piezo inhibitor composition may be present in any suitable container that is compatible with the Piezo inhibitor composition. By “compatible” is meant that the container is substantially inert (e.g., does not significantly react with) the liquid and/or reagent(s) of the Piezo inhibitor composition in contact with a surface of the container. Containers of interest may vary and may include but are not limited to a test tube, centrifuge tube, culture tube, falcon tube, microtube, Eppendorf tube, specimen collection container, specimen transport container, and syringe.

The container for holding the Piezo inhibitor composition may be configured to hold any suitable volume of the Piezo inhibitor composition. In some cases, the size of the container may depend on the volume of Piezo inhibitor composition to be held in the container. In certain embodiments, the container may be configured to hold an amount of Piezo inhibitor composition ranging from 0.1 mg to 1000 mg, such as from 0.1 mg to 900 mg, such as from 0.1 mg to 800 mg, such as from 0.1 mg to 700 mg, such as from 0.1 mg to 600 mg, such as from 0.1 mg to 500 mg, such as from 0.1 mg to 400 mg, or 0.1 mg to 300 mg, or 0.1 mg to 200 mg, or 0.1 mg to 100 mg, 0.1 mg to 90 mg, or 0.1 mg to 80 mg, or 0.1 mg to 70 mg, or 0.1 mg to 60 mg, or 0.1 mg to 50 mg, or 0.1 mg to 40 mg, or 0.1 mg to 30 mg, or 0.1 mg to 25 mg, or 0.1 mg to 20 mg, or 0.1 mg to 15 mg, or 0.1 mg to 10 mg, or 0.1 mg to 5 mg, or 0.1 mg to 1 mg, or 0.1 mg to 0.5 mg. In certain embodiments, the container is configured to hold an amount of Piezo inhibitor composition ranging from 0.1 g to 10 g, or 0.1 g to 5 g, or 0.1 g to 1 g, or 0.1 g to 0.5 g. In certain instances, the container is configured to hold a volume (e.g., a volume of a liquid Piezo inhibitor composition) ranging from 0.1 ml to 200 ml. For instance, the container may be configured to hold a volume (e.g., a volume of a liquid) ranging from 0.1 ml to 1000 ml, such as from 0.1 ml to 900 ml, or 0.1 ml to 800 ml, or 0.1 ml to 700 ml, or 0.1 ml to 600 ml, or 0.1 ml to 500 ml, or 0.1 ml to 400 ml, or 0.1 ml to 300 ml, or 0.1 ml to 200 ml, or 0.1 ml to 100 ml, or 0.1 ml to 50 ml, or 0.1 ml to 25 ml, or 0.1 ml to 10 ml, or 0.1 ml to 5 ml, or 0.1 ml to 1 ml, or 0.1 ml to 0.5 ml. In certain instances, the container is configured to hold a volume (e.g., a volume of a liquid Piezo inhibitor composition) ranging from 0.1 ml to 200 ml.

The shape of the container may also vary. In certain cases, the container may be configured in a shape that is compatible with the assay and/or the method or other devices used to perform the assay. For instance, the container may be configured in a shape of typical laboratory equipment used to perform the assay or in a shape that is compatible with other devices used to perform the assay. In some embodiments, the liquid container may be a vial or a test tube. In certain cases, the liquid container is a vial. In certain cases, the liquid container is a test tube.

As described above, embodiments of the container can be compatible with the Piezo inhibitor composition in contact with the reagent device. Examples of suitable materials for the containers include, but are not limited to, glass and plastic. For example, the container may be composed of glass, such as, but not limited to, silicate glass, borosilicate glass, sodium borosilicate glass (e.g., PYREX™), fused quartz glass, fused silica glass, and the like. Other examples of suitable materials for the containers include plastics, such as, but not limited to, polypropylene, polymethylpentene, polytetrafluoroethylene (PTFE), perfluoroethers (PFE), fluorinated ethylene propylene (FEP), perfluoroalkoxy alkanes (PFA), polyethylene terephthalate (PET), polyethylene (PE), polyetheretherketone (PEEK), and the like.

In some embodiments, the container may be sealed. That is, the container may include a seal that substantially prevents the contents of the container from exiting the container. The seal of the container may also substantially prevent other substances from entering the container. For example, the seal may be a water-tight seal that substantially prevents liquids from entering or exiting the container, or may be an air-tight seal that substantially prevents gases from entering or exiting the container. In some instances, the seal is a removable or breakable seal, such that the contents of the container may be exposed to the surrounding environment when so desired, e.g., if it is desired to remove a portion of the contents of the container. In some instances, the seal is made of a resilient material to provide a barrier (e.g., a water-tight and/or air-tight seal) for retaining a sample in the container. Particular types of seals include, but are not limited to, films, such as polymer films, caps, etc., depending on the type of container. Suitable materials for the seal include, for example, rubber or polymer seals, such as, but not limited to, silicone rubber, natural rubber, styrene butadiene rubber, ethylene-propylene copolymers, polychloroprene, polyacrylate, polybutadiene, polyurethane, styrene butadiene, and the like, and combinations thereof. For example, in certain embodiments, the seal is a septum pierceable by a needle, syringe, or cannula. The seal may also provide convenient access to a sample in the container, as well as a protective barrier that overlies the opening of the container. In some instances, the seal is a removable seal, such as a threaded or snap-on cap or other suitable sealing element that can be applied to the opening of the container. For instance, a threaded cap can be screwed over the opening before or after a sample has been added to the container.

As used herein, a “tissue disrupting device” is a device that causes cellular damage or injury. The tissue disrupting device may be configured to form a wound in a dermal location of a subject, e.g., according to any of the methods described herein. In some cases, the device may apply to a dermal location one or more of, e.g., ultrasound, radio frequency (RF), laser, ultraviolet energy, infrared energy, or mechanical disruption. Suitable tissue disrupting devices include, but are not limited to, surgical instruments (e.g., scalpels, lancets, etc.), needles, microneedles (e.g., a Dermaroller®), lasers, etc. In certain embodiments, the devices include 1, 2, 3, 4, 5, 6, 7, 8, 9, or 10 skin-penetrating component(s) (e.g., a needle, a drill, a microauger, a tube comprising cutting teeth, a spoon bit, a wire, a fiber, a blade, a high-pressure fluid jet, a cryoprobe, a cryoneedle, an ultrasound needle, a multi-hole needle including one or more chemical agents, a microelectrode, and/or a vacuum, or any other component described herein) that can penetrate the skin simultaneously. In some cases, the tissue disrupting device is configured to administer or deliver an effective amount of a Piezo inhibitor composition to a wound, e.g., a wound formed by the tissue disrupting device. In certain embodiments, the tissue disrupting device is configured to administer, e.g., inject, the Piezo inhibitor composition to a topical dermal location or below a topical dermal location of the subject. The administration may be performed with any suitable mechanism or medium according to any of the embodiments described above such as, e.g., a needle, microneedle, gel, etc. In some cases, one or more portions of the tissue disrupting device contains an effective amount of a Piezo inhibitor composition. In some cases, the tissue disrupting device includes one or more microneedles. In some cases, the tissue disrupting device includes an array of microneedles. In certain embodiments, the tissue disrupting device is a microneedling device including, e.g., the Dermaroller® or Dermapen®. In some cases, the tissue disrupting device is a laser, e.g., for practicing fractional laser resurfacing.

### Utility

The subject methods find use in applications involving wound healing including, e.g., clinical and research applications. In certain embodiments, the methods find use in postnatal wound healing or wound healing in adults. The methods also find use in reversing existing scarring, e.g., of healed wounds/existing scars. The methods may find use in any applications where a wound is intentionally, e.g., via surgery, or unintentionally created. Methods of embodiments of the invention also find use in ameliorating, e.g., reducing or inhibiting, organ fibrosis, e.g., liver fibrosis, heart fibrosis, inflammatory bowel fibrosis, muscle fibrosis, kidney fibrosis, etc., in a subject.

In certain embodiments, the subject methods find use in applications where it is desirable to reduce or prevent scarring, or reverse existing scarring. The subject methods may be applied to the treatment of all types of skin, including wound zones and eyes, where scarring is a possibility. In certain embodiments, the methods may be used to treat or prevent scarring of human skin resulting from burns, scalds, grazes, abrasions, cuts and other incisional wounds, surgery and pathological skin scarring conditions such as, e.g., Dupuytren's disease, and the conditions of fibrotic dermal scarring, hypertrophic scarring, keloid scarring and corneal and other ocular tissue scarring.

The subject methods further find use in applications for promoting hair growth. The subject methods may find use in applications where increased hair growth in a particular dermal location is desired, e.g., a region of substantial hair loss. In certain embodiments, the methods find use in treating hair loss and conditions involving hair loss as a side effect. The methods may be used to treat hair loss from a variety of conditions, such as, but not limited to hormonal changes during pregnancy and childbirth, disease (hyper- and hypo-thyroidism, lupus, trichotillomania), medication, chemotherapy, dietary deficiencies, stress, alopecia, trauma, radiotherapy, iron deficiency or other nutritional deficiencies, autoimmune diseases and fungal infection. In certain embodiments, the subject methods find use in treating a subject for alopecia.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### I. Piezo Mechanosensing Regulates Adipocyte Fate to Drive Fibrosis in Scarring

### A. SUMMARY

Skin is intimately associated with underlying fatty (adipose) tissue. While past studies have suggested that plasticity exists between dermal fibroblasts and adipocytes, (Shook et al., 2020) it remains unknown whether fat actively contributes to fibrosis in scarring. Here we show that adipocytes convert to scar-forming fibroblasts in response to Piezo-mediated mechanosensing to drive wound fibrosis. Using multiple in vitro and in vivo models to track adipocytes' response to wound-related stimuli, we establish that substrate mechanics alone are sufficient to drive adipocyte-to-fibroblast conversion. Further, by leveraging clonal lineage tracing (Rainbow mice) in combination with scRNA-seq, we define a “mechanically naïve” fibroblast subpopulation that represents a transcriptionally intermediate state between adipocytes and scar fibroblasts. Finally, we show that inhibiting Piezo1 or Piezo2 yields regenerative healing by preventing adipocytes' activation to fibroblasts, both in mouse wounds and in a newly developed human xenograft wound model. Importantly, Piezo1 inhibition induced dramatic wound regeneration even in pre-existing scars, a finding that suggests a role for adipocyte-to-fibroblast transition in wound remodeling, the least-understood phase of wound healing. Collectively, these results demonstrate that adipocyte-to-fibroblast transition plays a critical role in scarring, a finding with important translational implications. We highlight a novel mechanism through which tissue mechanics mediate skin fibrosis, wherein Piezo mechanosensitive ion channel activity specifically drives adipocyte differentiation to a pro-fibrotic fate. Adipocyte-to-fibroblast transition represents a relevant therapeutic target for minimizing fibrosis via Piezo inhibition in skin or other organs where associated fat may contribute to organ fibrosis.

### B. INTRODUCTION

Skin scarring is an acute fibrotic process that occurs following any injury to the adult dermis. Scarring affects over 100 million patients every year in the U.S. and can cause both visual disfigurement and functional impairment (e.g., growth restriction, joint contraction). (desJardins-Park et al., 2021; Griffin et al., 2020; Gurtner et al., 2008) The most downstream mediators of scarring are dermal fibroblasts, which deposit the excess, fibrotic extracellular matrix (ECM) comprising a scar. (Griffin et al., 2021; Gurtner et al., 2008) The potential contributions of subcutaneous adipose tissue to skin fibrosis are relatively undefined but of growing interest. (Sakers et al., 2022) Recent studies have revealed lineage plasticity between adipocytes and fibroblasts, suggesting that fibroblasts may be able to differentiate into adipocytes (Plikus et al., 2017) and vice versa. (Shook et al., 2020) Clinical and experimental correlates also implicate adipose tissue in the balance between fibrosis and regeneration: adipose tissue is lost in multiple fibrotic skin conditions (e.g., systemic sclerosis, (Marangoni and Lu, 2017) radiation fibrosis (Poglio et al., 2009)), whereas adipogenic differentiation has been associated with presence of neogenic hair follicles in large wounds. (Plikus et al., 2017) However, much remains unknown about adipocyte dynamics, including the function, molecular drivers, and effects of modulating adipocyte-to-fibroblast conversion in scarring.

Tissue mechanical forces are a critical mediator of scarring. This has long been recognized by surgeons, who incise along lines of relaxed skin tension (“Langer's lines”) to minimize postoperative scarring. (Wong et al., 2010) Experimentally, increasing tension across wounds increases scarring in mice; (Aarabi et al., 2007) conversely, inhibiting wound mechanics—using either a tension-offloading elastomeric dressing or small molecule inhibitors of mechanotransduction—has been shown to reduce scarring in both animal models and human clinical trials. (Kuehlmann et al., 2020) While it is well established that fibroblasts are highly mechanosensitive—for instance, we recently showed that mechanical forces activate a subset of dermal fibroblasts to adopt a pro-fibrotic phenotype (Mascharak et al., 2021a; Mascharak et al., 2020)—almost nothing is known about mechanoresponsiveness of dermal adipocytes. Rare in vitro studies suggest that adipocytes modify gene expression in response to substrate mechanics, (Hossain et al., 2010; Yuan et al., 2015) but understanding of adipocyte mechanical signaling remains extremely limited, and the effects of wound mechanical cues on adipocytes are entirely unknown.

Here, we apply multiple in vitro and in vivo models to accomplish lineage tracing of adipocytes and study their phenotypic response to substrate/wound mechanics. We show for the first time that mechanics can dramatically alter wound adipocyte fate: mechanical cues promote conversion of adipocytes into pro-fibrotic fibroblasts within wounds, via an intermediate, “mechanically naïve” fibroblast state. Further, blocking adipocyte mechanosensing, via blockade of Piezo1 or Piezo2 mechanosensitive ion channel components, prevents adipocyte-to-fibroblast transition and significantly decreases fibrosis and promotes wound regeneration, even in existing scars. Collectively, this study elucidates the role and molecular drivers of adipocyte-to-fibroblast conversion in wound repair and scarring, which represents a novel therapeutic target for preventing scarring and other fibroses.

### C. MATERIALS AND METHODS

### 1. Mice:

Transgenic mouse strains (acquired from Jackson Laboratories): Adipoq-cre/ERT2 (B6; 129-Adipoqtm1Chan/J, Stock: 08195), Col 1 (B6.Cg-Tg (Col1a1-cre/ERT2) 1Crm/J Stock: 016241), mTmG (B6.129 (Cg)-Gt (ROSA) 26Sortm4 (ACTB-tdTomato,-EGFP) Luo/J Stock: 007676), Piezo1^{fl/fl} (Piezo1tm2.1Apat/J Stock: 029213), Piezo2^{fl/fl} (B6 (SJL)-Piezo2tm2.2Apat/J, Stock: 027720), Piezo2-EGFP-IRES-Cre (B6 (SJL)-Piezo2tm1.1 (cre) Apat/J, Stock: 027719), Piezo1^{p1-tdT} (B6; 129-Piezo1tm1.1Apat/J, Stock: 029214), FAK^{fl/fl} (B6.129P2 (FVB)-Ptk2tm1.1Guan/J, Stock: 031956), YAP^{fl/fl} (Yap1tm1.1Dupa/J, Stock: 027929), ROSA26^{iDTR} (C57BL/6-Gt (ROSA) 26Sortm1 (HBEGF) Awai/J, Stock: 007900), CD1 (C.129S2-Cd1tm1Gru/J, Stock: 003814), B6 (C57BL/6J, Stock: 000664). Mice were housed at the Stanford University Comparative Medicine Pavilion per Stanford APLAC guidelines, under the supervision of the Veterinary Service Center (VSC). All animals were genotyped under Jackson Laboratory instructions, using Transnetyx's Automated Genotyping PCR services. ROSA26^{mTmG} mice utilize a dual-fluorescence reporter system that irreversibly substitutes Tomato red fluorescent protein (RFP) with membrane-bound green fluorescent protein (GFP) after recombination. Adipoq^{Cre-ERT2} mice were crossed with ROSA26^{mTmG} reporter mice to trace Adiponectin-lineage-positive cells, as defined by GFP expression. Rainbow (ROSA26^{VT2/GK3}) mice were gifted by the Weissman Laboratory, Stanford University School of Medicine.

### 2. Dorsal Excisional Wounding:

### Induction:

Intraperitoneal tamoxifen injections (90% corn oil/ethanol v/v; 200 mg/kg body weight) were used to induce Adipoq^{Cre/ERT2}; ROSA26^{mTmG} mice (n=6 mice per experimental group) every day for 5 consecutive days prior to surgery. Topical tamoxifen (150 μL) was applied for 3 consecutive days following surgery.

### Wounding:

For wounding, anesthesia was induced and maintained with 1-3% isoflurane at a flow rate of 2 L/min. Adequate anesthesia was confirmed with the loss of hind-limb reflex to nociceptive stimuli. Dorsal skin was sterilized with Betadine Surgical Scrub Veterinary (Avrio Health L.P.™, Stamford, CT) followed by sterile alcohol prep pads (FisherScientific™, Pittsburgh, PA). Next, four 6 mm full-thickness circular wounds were made through the panniculus carnosus with sterile scissors and forceps. Wounds were equally spaced on the dorsum of each animal at least 4 mm lateral to the midline and stented open using 10 mm diameter silicone rings. Rings were secured using Krazyglue™ and 6 simple interrupted nylon monofilament 4-0 sutures (Dynarex™, Orangeburg, NY). Wounds were dressed using 3M Tegaderm Transparent Film 1626w dressings (3M™, Cat: 1626W). Dressing changes took place every 48 hours under anesthesia. 200 ng topical diphtheria toxin (DT) in 30 μL PBS (or 30 μL PBS control) was injected intradermally every day over 3 days to ablate adipocytes in Adipoq^{Cre-ERT2/Awai} mice. For mice receiving treatments with inhibitors of mechanosensitive ion channels, treatments consisted of a single administration at POD 0, via local intradermal injections into the wound edge; PBS was injected for vehicle controls. 30 μL (0.2 mg/ml of inhibitor) was delivered per wound area of Piezo1 inhibitor (GsMTx4 [500 nM], Tocris, Cat: 4912) or Piezo 2 inhibitor (D-GsMTx4 [500 nM], Tocris, Cat: 4912) (n=6).

### Harvest:

Wounds were re-epithelialized by postoperative day 14 (POD 14), at which time the wounds were harvested with dissecting scissors and processed for histology. Dissection followed fascial planes. Surrounding skin was also harvested for use as unwounded controls. Mice were euthanized by CO₂ narcosis and cervical dislocation. Harvested skin for Fluorescence-activated cell sorting (FACS) was mechanically digested using dissecting scissors to finely mince each specimen. Harvested skin for use in histology and immunofluorescent (IF) staining was placed in tissue embedding cassettes.

### Transplant:

Fat pads were harvested from mTomato⁺ (mTmG) mice and underwent mechanical digestion as above. Tissue was then enzymatically digested in a Collagenase II and IV solution (1:1, 1500 U/ml in Dulbecco's Modified Eagle Medium [DMEM]). Samples were placed on an oscillating plate at 150 rpm for 40 minutes at 37° C. At the end of digestion, FACS buffer (see FACS section below for composition) was added to stop enzyme activity and solution was strained using a 70 μm cell strainer. Tomato⁺adipocytes were then injected intradermally into a wildtype (B6) mouse, which was prepared 24 hours prior to injection as described in the section above (Dorsal Excisional Wounding). Then, 48 hours after injection, wildtype mice (n=4) were wounded in the engrafted area. Harvests took place at POD 14.

### 3. Hypertrophic Scarring Model (HTS):

Mice were prepared as described in the section above (Dorsal Excisional Wounding). Linear incisions were made on the dorsum of the mice approximately 20 mm long. Incisions were closed using simple interrupted nylon monofilament 4-0 sutures (Dynarex™, Orangeburg, NY). A loading device consisting of 22 mm expansion screws and Luhr plate supports was placed over each wound on POD 4. The device was secured using Krazyglue™ and sutured in place. Tension was increased by 2 mm expansion of the loading device every 2 days for 10 days. Mice with attached devices and no expansion protocol, as well as incision only with no device attached, also served as controls. Both wounded and control samples were harvested on POD 18 (n=6).

### 4. Xenograft Model:

### Animals:

For the xenograft model, adult immunocompromised CD-1 nude mice were purchased from The Jackson Laboratory (Bar Harbor, ME). Mice were maintained at the Stanford University Research Animal Facility, SIM-1 Barrier Facility in accordance with Stanford University guidelines. All experiments were performed under an approved APLAC protocol (APLAC #11048). 3 mice were used for each group.

### Human Foreskin Samples:

Discarded postnatal human foreskin samples were collected after circumcision at the Lucille Packard Children's Hospital at Stanford under a Stanford University Institutional Review Board (IRB) approved protocol (IRB #45219). Samples were not used if collected >8 hours after circumcision; samples were kept in Dulbecco's Modified Eagle Medium (ThermoFisher Scientific™, Waltham, MA) on ice until grafting. Foreskin was prepared by removal of all subcutaneous tissue, including muscle, and divided into 1 cm sections to be grafted.

### Grafting Protocol:

Nude mice were induced and maintained with 2-4% isoflurane (Product: 502017, MWI Veterinary Supply Co.®; Boise, ID) at a rate of 2 L/min. While anesthetized, Betadine Surgical Scrub Veterinary (Avrio Health L.P.™, Stamford, CT) and sterile alcohol prep pads (FisherScientific™, Pittsburgh, PA) were used to prepare the dorsum. A 1.2 cm full-thickness excision of dorsal skin was created with forceps and sharp scissors. The foreskin graft was then placed into the full-thickness wound. 5-0 monofilament Nylon sutures (Medtronic, Minneapolis, MN) were circumferentially placed in a simple interrupted fashion and equal cross tension was maintained across the graft during placement. Tegaderm film dressings (3M™, Saint Paul, MN) served as pressure dressings post-operatively. Dressings were kept for five days post-operatively before being removed.

### Wounding Protocol:

At two weeks post-operatively, complete engraftment was established and the nylon sutures were removed. A 4 mm punch biopsy was then used to create a full thickness wound within the graft using the described sterile technique. For mice receiving treatments with inhibitors of mechanosensitive ion channels, treatments were administered via local intradermal injections into the wound edge; PBS was injected for vehicle controls. 30 μL (0.2 mg/ml of inhibitor) was delivered per wound area of Piezo1 and Piezo 2 inhibitors at POD 0. Wounds were dressed with Tegaderm film dressings (3M™, Saint Paul, MN) and changed every 48 hours for 5 days. Wounds were observed until POD 14 at which point they were harvested for analysis.

### 5. Wound Curve Analysis:

Gross measurements were taken using Adobe Photoshop 22.5.1 (Adobe Systems, San Jose, CA) to determine the percent closure of the wound until POD 14 (n=6). Scar size was measured relative to the inner circumference of the silicone ring. Percentage of the original wound is plotted from baseline day 0 to day 16.

### 6. EdU In Vivo:

Mice received intraperitoneal injections of EdU (5-ethynyl-2′-deoxyuridine) at a concentration of 100 mg/kg 24 hours before wounding and again 24 hours before euthanasia (n=6). For FACS analysis, cells were extracted using digestion methods previously described and stained using the Click-iT™ Plus EdU Alexa Fluor™ 647 Fluorescence-activated cell sorting Assay Kit (ThermoFisher, Cat: C10634). EdU incorporation was analyzed using a BD FACSAria II. For Immunohistochemical analysis of EdU incorporation in vivo, skin was harvested, fixed, and sectioned as previously described. Slides with skin sections were stained using Click-iT™ EdU Cell Proliferation Kit for Imaging, Alexa Fluor™ 647 dye (ThermoFisher, Cat: C10337), and fluorescent images of live cells were taken with an LSM880 inverted confocal microscope.

### 7. Tensile Strength Testing:

Skin from wounded and unwounded mice at day 14 were tested using an Instron 5565 using a 100 N load cell. Dorsal wounds were excised with sharp surgical scissors and carefully cut into tapered 4 mm by 15 mm pieces. Tissue pieces were subsequently anchored with grips such that the middle of the wound was positioned centrally between the grips. The tissue was slowly separated (1% increase per second) until failure (defined by a clear drop in measured stress as tension increased). Young's Modulus was determined by taking the slope of the linear portion of the stress-strain curve.

### 8. Harvesting Cells for Fluorescence-Activated Cell Sorting (FACS):

Following CO₂ euthanasia, dorsal skin wounds were dissected and washed once in PBS. Wounded tissue was then diced into a fine consistency using sharp surgical scissors. Minced skin tissue was then enzymatically digested using a 1:1 ratio of Collagenase Type IV (ThermoFisher, Cat: 17104019) and Collagenase Type II (ThermoFisher, Cat: 17101015) at a concentration of 1500 U/ml in DMEM (ThermoFisher, Cat: 10569010) for 90 minutes at 37° C. Samples were continuously agitated at 150 rpm during enzymatic digestion. Enzyme activity was stopped using FACS buffer, and digested tissue was strained through 70 μm cell strainers. Cells were pelleted at 1500 rpm for 5 minutes at 4 C and then resuspended in 150 μL of FACS buffer for primary antibody staining. For lineage negative (Lin−) FACS analysis, the following primary antibodies were used: Tie2 (ThermoFisher, Cat: 13598782), CD45 (ThermoFisher, Cat: 48045182 or ThermoFisher, Cat: 13045181), CD31 (ThermoFisher, Cat: RM52280 or ThermoFisher 13031181), CD324 (ThermoFisher, Cat: 13324982), CD326 (ThermoFisher, Cat: 45592185), Ter119 (ThermoFisher, Cat: 14592182). For fibroblast subpopulation analysis, the following primary antibodies were used: D1k1 (R&D systems, Cat: FAB8635A or FAB8634N), Sca1 (Ly-6A/E) (BioLegend, Cat: 108133), and CD26 (ThermoFisher, Cat: 45026182 or Biolegend 137809). Cells were stained with primary for 30 minutes on ice. Following primary antibody staining, samples were washed with 500 μL of FACS buffer, spun at 1500 rpm for 5 minutes (4 C), and resuspended in 150 μL of FACS buffer for secondary antibody staining using either Streptavidin-eFluor450 (ThermoFisher, Cat: 48431782) or Streptavidin-Alexa Fluor 647 (S21374). Cells were stained with secondary for 20 minutes on ice. Cells were washed again with 500 μL of FACS buffer, and DAPI (4′,6-diamidino-2-phenylindole) (Biolegend, Cat: 422801) was added to label dead cells. A BD II FACS Aria machine was used for FACS sorting and analysis.

### 9. Histology and Immunofluorescent Staining:

### Fixation:

Fluorescent tissues from transgenic mice were fixed in 4% paraformaldehyde (PFA) solution in PBS (Electron Microscopy Sciences, Cat: 15710) for 24 h at 4° C. All other samples were fixed in 10% neutral buffered formalin (NBF; ThermoFisher Scientific™, Waltham, MA) for 24 hours at room temperature.

### Cryosectioning:

Fixed samples were soaked in 30% sucrose dissolved in PBS at 4° C. After one week, samples were removed from the sucrose solution and embedded as tissue blocks using Tissue Tek O.C.T. (Sakura Finetek, Torrance, CA) over dry ice and 100% ethanol to achieve rapid freezing. Frozen blocks were mounted on a Thermo Scientific CryoStar NX70 cryostat, and 8 μm-thick sections were transferred to Superfrost/Plus adhesive slides (ThermoFisher Scientific™, Waltham, MA).

### Paraffin Sectioning:

Automated Tissue Processor (ThermoFisher Scientific™, Waltham, MA) was used to dehydrate samples in a gradient of alcohols. Tissue was then embedded using ThermoFisher Histostar Tissue Embedding station. Paraffin blocks were trimmed as necessary and cut as 8 μm-thick sections. Paraffin ribbons were placed in a water bath at 40° C. and mounted onto Superfrost/Plus adhesive slides (ThermoFisher Scientific™, Waltham, MA). Sections were baked at 50° C. overnight.

### Staining:

Hematoxylin and eosin (Cat: H-3502; Vector Laboratories, Burlingame, California), Masson's Trichrome (ab150686; Abcam®, Waltham, MA), Picro-sirius Red (ab150681; Abcam®, Waltham, MA), and Oil Red O (Sigma-Aldrich™, St. Louis, MO) stains with standard protocols were used. Cryosection samples were first dehydrated using a slide rack, submerged into 1% PBS for 10 minutes, followed by 30% ethanol (EtOH), 50% EtOH, 70% EtOH, 95% EtOH, and 100% EtOH for 15 minutes each. Paraffin sections were hydrated prior to staining by placement in xylene for 20 minutes, followed by 10 minutes each of 100% EtOH, 95% EtOH, 70% EtOH, 50% EtOH, and 30% EtOH. Slides were then submerged in running tap water for 10 minutes.

For immunofluorescent staining, slides were washed twice in Tween 20 (Sigma-Aldrich™, St. Louis, MO) followed by one wash in PBS. Slides were then blocked for 1 hour with Power Block (Biogenex™, Fremont, CA) prior to addition of the following primary antibodies: Abcam ab40794 (anti-FAK), Abcam ab3526 (anti-perilipin), Abcam ab5694 (anti-α-SMA), Abcam ab181595 (anti-CK14), Abcam ab52625 (anti-CK19), Abcam ab59436 (anti-collagen type III), Abcam ab51317 (anti-Sca1), Abcam ab197896 (anti-S100A4), ThermoFisher Scientific MA1-26771 (anti-collagen type I), ThermoFisher Scientific PA5-16571 (anti-PDGFRα), ThermoFisher Scientific PA3-821A (anti-PPARγ), R&D systems anti-mAcrp30 (anti-adiponectin), Proteintech 15939-1-AP (anti-Piezo1), Novus Biologicals NBP1-78624 (anti-Piezo2), Santa Cruz Biotechnology SC-101199 (anti-YAP1), Santa Cruz Biotechnology SC-7309 (anti-CD36), Sigma-Aldrich AB9260 (anti-Ki-67).

Slides were then incubated for 1 h with Alexa Fluor 488, 594, or 647-conjugated anti-rabbit, anti-rat, or anti-mouse antibodies (Invitrogen, Waltham, MA). Finally, slides were mounted in Fluoromount-G mounting solution with or without DAPI (ThermoFisher Scientific™, Waltham, MA). Brightfield images were acquired with a Leica CTR4000 microscope, while fluorescent images were acquired with a LSM880 inverted confocal, Airyscan, AiryscanFAST, GaAsP detector upright confocal microscope.

Hematoxylin and eosin staining started with submerging slides into Hematoxylin for 10 minutes. Slides were then submerged into tap water for 5 minutes, then dipped into eosin 12 times. DI water baths were prepared and slides were submerged until water was clear of eosin. Slide racks were then dipped into 70% EtOH 10 times, followed by 1 minute in 95% EtOH then 100% EtOH. Finally slides were dipped into Xylene 8 times until they were mounted on Superfrost/Plus adhesive slides (ThermoFisher Scientific™, Waltham, MA) with Permount Mounting Medium (Electron Microscopy Sciences™, Hatfield, PA).

For Trichrome staining, Bouin's solution was added to the samples for 60 minutes in a humidity chamber. To create humidity chambers, slide boxes were lined with wet paper towels. Next, slides were submerged into running tap water for 5 minutes. Working Weigert's Iron Hematoxylin was then added to samples for 5 minutes, followed by submersion into running tap water for 5 minutes. Biebrich Scarlet/Acid Fuchsin Solution was then added to samples for 4 minutes. After slides were submerged in running tap water for 5 minutes, Phosphomolybdic/Phosphotungstic Acid was added to the sample for 45 minutes. Phosphomolybdic/Phosphotungstic Acid was removed from slides and Aniline Blue Solution was added for 4 minutes without a washing step. After Aniline Blue staining, slides were submerged into running tap water for 5 minutes. Correct staining was confirmed using Leica CTR4000 microscope. Finally, slides were dipped into 1% Glacial Acetic Acid Solution then running water 12 times each, followed by 15 dips each in 95% EtOH then 100% EtOH. Slides were submerged into Xylene 8 times prior to mounting with Permount.

Dehydration/rehydration steps were not completed for Picrosirius red staining. Slides were first washed 3 times with PBS and then submerged into running tap water for 1 minute. Picrosirius red was added to slides for 60 minutes in a humidity chamber, previously described. After the completion of the 1 hour stain, slides were dipped into 2 different changes of 0.5% Glacial Acetic Acid 10 times (20 dips total), followed by 10 times into 2 different changes of 100% ETOH (20 dips total), and 8 times into Xylene. Slides were then mounted with Permount.

### Picrosirius Red Stained Histologic Analysis:

Analysis of picrosirius red stained tissue sections took place using an image-processing algorithm. The algorithm profiles 26 ultrastructural features to provide a quantitative comparison of extracellular matrices.¹¹ Each group (n=6) was randomly imaged at 100 separate locations at 40×. Color deconvolution following previously described methods (Ruifrok and Johnston, 2001) was performed to characterize each stain by absorbance in three RGB channels. Ortho-normal transformation was then used to determine each color's contribution to the captured image. Red and green images were produced, representing mature and immature ECM fibers, and analyzed as separate groups. A Matlab script was used to achieve analysis, including noise reduction, preferential selection for smooth regions with low variance, and “skeletonization” of images to characterize the fiber networks. The algorithm also allowed for measurement of fiber length, width, persistence, alignment, and overall dimensionality.

Oil Red O Staining was completed using frozen sections. Sections were first fixed in formalin for 15 minutes, followed by a wash step in running tap water for 5 minutes. Slides were then rinsed in 60% isopropanol prior to a 15-minute staging in Oil Red O working solution. Working solution was made from 30 ml of the stock stain and 20 ml of distilled water. Stock stain was made using 0.5 g of Oil Red O (Sigma-Aldrich™, St. Louis, MO) dissolved in 100 ml of isopropanol using a warm water bath. Filter paper was then used to add stock stain to a 50 ml conical. Stains were completed on the same day that a working solution was made using Coplin Staining Jars.

### 10. RNAscope:

RNAscope® 2.5 HD Assay in situ hybridization assay was conducted starting with a permeabilization step. Paraffin-embedded tissue sections were pretreated to permeabilize cells and expose target RNA. Next, RNAscope® and double-Z design probes were hybridized to the target sequence. Signals were amplified using detection reagents and labeled fluorescent probes were added to bind to each amplifier region. Target probes, amplifiers and label probes were sourced from Advanced Cell Diagnostics, Hayward, CA. Samples were imaged using a bright field microscope with a Leica CTR4000 microscope and positive pixels were quantified in Imagej.

### 11. Cell Culture:

### Mouse Cell Line:

Embryonic fibroblast cell line 3T3-L1 (CL-173, ATCC) was purchased and revived per manufacturer instructions. Following revival, cell expansion was monitored, and media was replaced every other day. Cells were passaged once confluency exceeded 70%. Cells used for experiments were between passages 2-5. Fibroblasts were cultured in DMEM+Glutamax media (ThermoFisher, Cat: 10569010) enriched with 10% fetal bovine serum (ThermoFisher, Cat: 10082147) and 1% Antibiotic-Antimycotic (ThermoFisher, Cat: 15240062) at 37° C. and 5% CO2.

### Human Primary Adipocytes:

Tissue was collected according to approved Stanford University IRB protocols. Human Lipoaspirate samples were washed in PBS and minced using sterile scissors. Samples were then enzymatically digested using Collagenase Type I (ThermoFisher, Cat: 17018029) at a concentration of 1500 U/ml in DMEM (ThermoFisher, Cat: 10569010) for 45 minutes at 37° C. Samples were continuously agitated at 150 rpm during enzymatic digestion. Enzyme activity was stopped using fetal bovine serum (FBS) enriched media, and digested tissue was strained through 300 um and 100 um cell strainers successively. Filtered samples were then spun at 1500 rpm for 5 minutes at 4° C. Mature adipocytes were isolated from the supernatant.

### 12. Differentiation of Embryonic Fibroblast Cells to Adipocytes:

Embryonic fibroblast cell line 3T3-L1 (CL-173, ATCC) was treated with Adipogenic medium containing DMEM+Glutamax media (ThermoFisher, Cat: 10569010) enriched with 10% fetal bovine serum (ThermoFisher, Cat: 10082147), 1% Antibiotic-Antimycotic (ThermoFisher Cat: 15240062), 10 ng/ml Insulin (Sigma, Cat: 0516), 500 mM 3-isobutyl-1-methylxanthine (FisherScientific, Cat: AC228420010), 1 mM Dexamethasone (Sigma, Cat: 1756), and 1 mM Rosiglitazone (Stem Cell Technologies, Cat: 72622). Cells were incubated at 37° C. and 5% CO₂. Cells were treated for 10 days and media was replaced every other day. Adipogenic differentiation protocol was based on published protocol (Guasti et al., 2012) and was validated in this study via ICC. In order to validate the differentiation protocol, embryonic fibroblast cells (3T3-L1, ATCC) were seeded in 24 well plates at 20,000 cells per well. Once confluent, wells received either adipogenic differentiation media or standard fibroblast media for 10 days. Cells were then fixed (4% PFA for 24 hours at 4° C.) immunocytochemically stained with anti-Adiponectin (Novus Biologicals, Cat: AF1119) and anti-Collagen I (ThermoFisher Cat: MA1-26771) antibodies. Fluorescent images were taken with a LSM880 inverted confocal microscope.

### 13. Proliferation and Apoptosis:

P1i and P2i treated adipocytes (and fibroblast controls) were examined for treatment toxicity using proliferation and apoptosis assays. Embryonic fibroblast cells (3T3-L1, ATCC) were seeded in 24 well plates at 20,000 cells per well. Once confluent, wells received either adipogenic differentiation media or standard fibroblast media for 10 days. Then, cells were treated with either 0.5×, 1×, or 2× the standard concentration of Piezo1 or Piezo2 inhibitors [500 nm]. After 7 days of treatment, cells were fixed or harvested for analysis of potential toxicity. For proliferation, EdU (5-ethynyl-2′-deoxyuridine) incorporation in vitro was investigated on differentiated adipocytes and undifferentiated fibroblasts using the Click-iT™ EdU Cell Proliferation Kit for Imaging, Alexa Fluor™ 488 dye (ThermoFisher, Cat: C10337). Proliferation was detected according to the manufacturer's guidelines. To evaluate apoptosis, the Annexin V Apoptosis Detection Kit-AF647 (Abcam, Cat: 219919) was used according to the manufacturer's guidelines.

### 14. Gels:

### Human Gels:

Human Adipocytes (collected and processed according to Cell Culture-Human Primary Adipocytes) were used to generate adipocyte-populated collagen I hydrogels as per a previously published protocol. (Chen et al., 2018) In short, a solution of 2 mg/mL collagen I (Advanced Biomatrix, Cat: 5005), 0.8×MEM (ThermoFisher, CAT: 11430030) in 16 mM HEPES (Sigma, CAT: 83264), and suspended human adipocytes (concentration 250,000 cells/mL) in DMEM (ThermoFisher, Cat: 10569010) were pipetted into cruciform-shaped PDMS (DOW, Cat: 2646340) molds and allowed to gelate before removing the mold. Hydrogels were constrained by metal pins and fibroblast media containing DMEM+Glutamax media (ThermoFisher, Cat: 10569010) enriched with 10% fetal bovine serum (ThermoFisher, Cat: 10082147) and 1% Antibiotic-Antimycotic (ThermoFisher Cat: 15240062) was added. After 24 hours, formed hydrogels were subjected to either 10% equibiaxial strain or no strain. Strain was achieved through movement of metal anchor pins in each of the four arms of the hydrogel. Strain quantification was confirmed by Fiji analysis of TiO₂ dye marks on the central region of each hydrogel. Media containing small molecule mechanotransduction inhibitors to Piezo1 (GsMTx4 [500 nM], Tocris, Cat: 4912) Piezo2 (D-GsMTx4 [500 nM], Tocris, Cat: 4912), FAK (PF573228 [10 uM], Tocris, Cat: 3239) YAP (Verteporfin [300 nM], Sigma, Cat: SML0534), or Vehicle Control (PBS) was added. Media was replaced every other day. Hydrogels were kept for 7 days. For IHC analysis of gel contents, gels were fixed whole in 4% PFA (ChemCruz, Cat: 281692) after being washed in PBS. For FACS and PCR analysis, cells were harvested by enzymatic digestion using Collagenase Type I (ThermoFisher, Cat: 17018029) at a concentration of 1500 U/ml in DMEM (ThermoFisher, Cat: 10569010) for 25 minutes at 37° C. Enzyme activity was stopped using FBS enriched media. Cells were spun, and the pellet was processed for FACS or resuspended in TRIzol for PCR.

### Mouse Gels:

Mouse fibroblasts (collected according to Cell Culture-Mouse Cell line) were used to generate adipocyte-populated collagen I hydrogels as per a previously published protocol. (Chen et al., 2018) In short, a solution of 2 mg/mL collagen I (Advanced Biomatrix, Cat: 5005), 0.8×MEM (ThermoFisher, Cat: 11430030) in 16 mM HEPES (Sigma, CAT: 83264), and suspended mouse adipocytes (concentration 500,000 cells/mL) in DMEM (ThermoFisher, Cat: 10569010) were pipetted into cruciform-shaped PDMS (DOW, Cat: 2646340) molds and allowed to gelate before removing the mold. Hydrogels were constrained by metal pins and Adipogenic medium containing DMEM+Glutamax media (ThermoFisher, Cat: 10569010) enriched with 10% fetal bovine serum (ThermoFisher, Cat: 10082147), 1% Antibiotic-Antimycotic (ThermoFisher Cat: 15240062), 10 ng/ml Insulin (Sigma, Cat: 0516), 500 mM 3-isobutyl-1-methylxanthine (FisherScientific, Cat: AC228420010), 1 mM Dexamethasone (Sigma, Cat: 1756), and 1 mM Rosiglitazone (Stem Cell Technologies, Cat: 72622) was added. After 10 days of differentiation, formed hydrogels were subjected to either 10% equibiaxial strain or no strain. Strain was achieved through movement of metal anchor pins in each of the four arms of the hydrogel. Strain quantification was confirmed by Fiji analysis of TiO₂ dye marks on the central region of each hydrogel. Media containing small molecule mechanotransduction inhibitors to Piezo1 (GsMTx4 [500 nM], Tocris, Cat: 4912) Piezo2 (D-GsMTx4 [500 nM], Tocris, Cat: 4912), FAK (PF573228 [10 uM], Tocris, Cat: 3239) YAP (Verteporfin [300 nM], Sigma, Cat: SML0534), or Vehicle Control (PBS) was added. Media was replaced every other day. Hydrogels were kept for 7 days. For ICC analysis of gel contents, gels were fixed whole in 4% PFA (ChemCruz, Cat: 281692) after being washed in PBS. For FACS and PCR analysis, cells were harvested by enzymatic digestion using Collagenase Type I (ThermoFisher, Cat: 17018029) at a concentration of 1500 U/ml in DMEM (ThermoFisher, Cat: 10569010) for 25 minutes at 37° C. Enzyme activity was stopped using FBS enriched media. Cells were spun, and the pellet was processed for FACS or resuspended in TRIzol for PCR.

### Immunocytochemistry Processing for Gels:

Following 24 hour fixation, gels were carefully dissected into 2 mm×2 mm pieces and placed into wells of a 96 well plate. Gentle agitation was provided to the plates during staining via a platform shaker at 25° C. Gel pieces were washed in PBS for 30 minutes. Gels were then permeated with 0.1% TritonX-100 in PBS (ThermoFisher, Cat: 85111) for 30 minutes. Gels were then blocked with PowerBlock (BioGenex, Cat: HK085-GP) for 1 hour. Following blocking, primary antibody was made up in a 1:1 solution of 0.1% TritonX-100 and PowerBlock. Primary staining lasted 1 hour, followed by two 15 minute washes in 0.025% Tween 20 in PBS (Sigma, Cat: P1379) and a single 15-minute wash in PBS. Secondary antibody containing Alexa Fluor 488, 594, or 647 against the primary antibody was added to a 1:1 solution of 0.1% TritonX-100 and Powerblock. Secondary antibody staining lasted 45 minutes, followed by two 30 minute washes in 0.025% Tween 20 in PBS (Sigma, Cat: P1379) and a single 30-minute wash in PBS. Finally, gel pieces were mounted on a slide with Fluoromount-G mounting solution with DAPI (ThermoFisher Cat: 00495952). Fluorescent images were taken with an LSM880 inverted confocal microscope.

The following primary antibodies were used for staining the gels: Abcam ab40794 (anti-FAK), Abcam ab3526 (anti-perilipin), ThermoFisher Scientific MA1-26771 (anti-collagen type I), R&D systems anti-mAcrp30 (anti-adiponectin), Proteintech 15939-1-AP (anti-Piezo1), Novus Biologicals NBP1-78624 (anti-Piezo2), Santa Cruz Biotechnology SC-101199 (anti-YAP1), Santa Cruz Biotechnology SC-7309 (anti-CD36).

### Calcium Imaging of Gels:

Calcium imaging was used to determine in vitro Ca²⁺ flow in hydrogel-bound adipocytes with or without mechanical strain and with or without Piezo1 inhibitor treatment. A Fluo-4, AM cell permeant kit (ThermoFisher, Cat: F-14201) was used per the manufacturer's instructions. Fluorescent images of live cells were taken with an LSM880 inverted confocal microscope. Imagej was used for image quantification.

shRNA Knockdown of Piezo1 in Gels:

Short hairpin RNA (shRNA) knockdown of Piezo1 gene expression in human primary adipocytes was performed per manufacturer guidelines (Santa Cruz, US). Human primary adipocytes were collected as described in Cell Culture-Human Primary Adipocytes and populated into collagen 1 hydrogels as described in Gels-Human Gels. Twenty-four hours after the formation of the gels, shRNA knockdown, scrambled control, or vehicle control was delivered. Following knockdown, gels were subjected to 0 or 10% equibiaxial strain. Seven days after strain, gels were harvested for FACS analysis or fixed in 4% PFA for Immunocytochemistry. Other than replacing traditional culture conditions in flasks/wells with a hydrogel populated system, no other modifications were made to the manufacturer's protocol.

### ELISA:

Media samples from mouse adipocyte-populated collagen 1 hydrogels, human adipocyte-populated collagen 1 hydrogels, Mouse Adiponectin, Mouse Collagen 1, Human Collagen 1, and Human Adiponectin were tested using Abcam mouse/human enzyme linked immunoassay (ELISA) kits (Abcam, Cat: ab108785, ab210579, ab229389, USA) according to manufacturer's protocol. Standards and samples were placed into wells coated with Collagen 1 or Adiponectin specific antibodies. Enzyme-linked polyclonal antibodies were then added to wells, followed by the addition of substrate. A spectrophotometer captured absorbances, and concentrations of protein target were calculated and modeled using Prism.

### 15. RT-qPCR:

RNeasy mini kit (Qiagen LLC, Germantown MD) was used for RNA extraction for real time quantitative polymerase chain reaction (qPCR). Transcription was then performed using Moloney murine leukemia virus reverse transcriptase. Following standard manufacturers' protocols, an ABI Prism PCR7500 sequence detection system (Applied Biosystems, Waltham, MA) using TaqMan expression assays (ThermoFisher CA).

Primer sequences were used as follows:

| Mouse | Human |
|---|---|
| Adipoq: Mm00456425_m1 | ADIPOQ: Hs00605917_m1 |
| Col1a1: Mm00801666_g1 | COL1A1: Hs00164004_m1 |
| Gapdh: Mm99999915_g1 | GAPDH: Hs02786624_g1 |

### 16. Single-Cell Sequencing:

Dermal wounds were harvested at POD 14 and mechanically digested with sharp dissecting scissors (n=6 per condition, performed in 3 independent experiments). Tissue was then added to an enzymatic digestion consisting of Collagenase II (ThermoFisher, Cat: 17101015) and IV (ThermoFisher, Cat: 17104019) in DMEM-F12 (GIBCO™, Fischer Scientific, Hampton, NH). Samples were added to an orbital shaker at 150 rpm for 90 minutes at 37° C. FACS buffer was added to quench the digest and samples were filtered through 70 μm cell strainers. Samples were then centrifuged at 1500 for 5 minutes at 4° C. Cell suspensions were labeled with TotalSeq Series B hashtag oligonucleotide-labeled antibodies (BioLegend). Samples were then centrifuged and resuspended with 0.04% UltraPure BSA (Thermo Fisher, Waltham, MA); cell counts were completed. A second filtration step took place using 40 μm cell strainers. Quality control and single cell RNAseq were performed on unsorted cells using the 10× Chromium Single Cell platform (Single Cell 3′ v3, 10× Genomics, USA) at the Stanford Functional Genomics Facility (SFGF), Stanford University, Palo Alto.

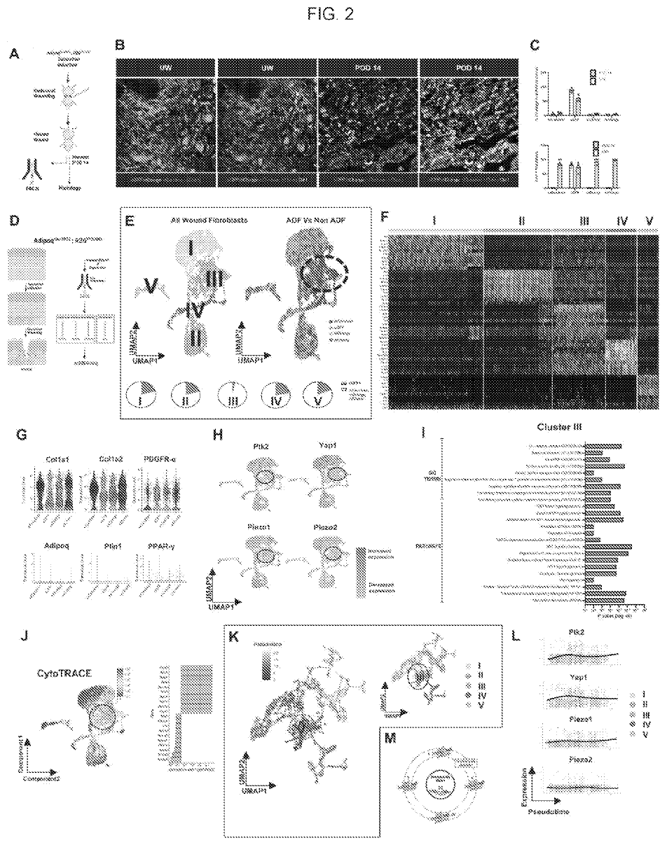

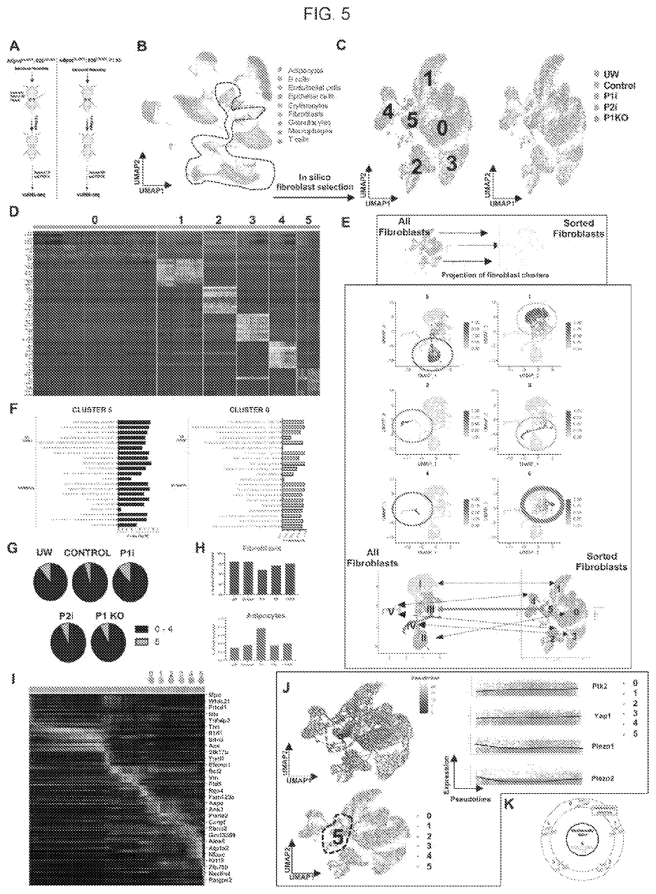

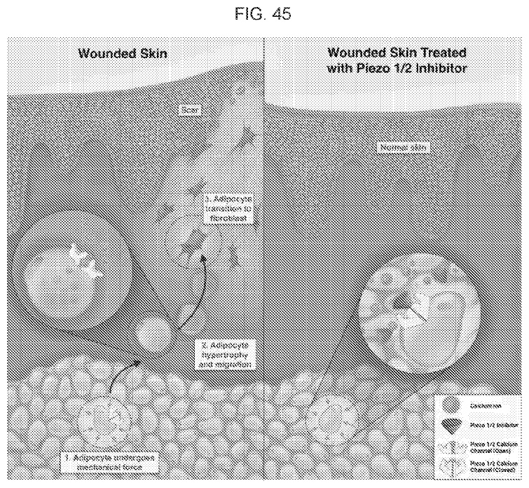

Base calls were converted to reads using the Cell Ranger (10× Genomics; version 3.1) implementation mkfastq and then aligned against the Cell Ranger mm10 reference genome, available at: http://cf.10×genomics.com/supp/cell-exp/, using Cell Ranger's count function with SC3Pv3 chemistry and 5,000 expected cells per sample, as previously described. (Mascharak et al., 2020) Hashtag oligos (HTOs) for samples were demultiplexed using Seurat's implementation HTODemux as previously described. (Mascharak et al., 2020) For both datasets (FIG. 2 and FIG. 5), a maximum percent mitochondrial RNA cutoff of 15% was employed. (Mascharak et al., 2022) For FIG. 2, cutoffs of 7,500 maximum unique genes and 85,000 maximum RNA counts were used. For FIG. 5, cutoffs of 6,500 maximum unique genes and 65,000 maximum RNA counts were used. This resulted in 4,116 cells for FIG. 2 and 45,725 cells for FIG. 5 (12,041 of which were later classified as Fibroblasts).

Unique molecular identifiers (UMIs) from each cell barcode were retained for all downstream analysis, normalized with a scale factor of 10,000 UMIs per cell, and subsequently natural log transformed with a pseudocount of 1 using the R package Seurat (version 4.0.5). (Chen et al., 2013) The first 15 principal components of the aggregated data were then used for uniform manifold approximation and projection (UMAP) analysis. (Gulati et al., 2020) Cell annotations were ascribed using SingleR (version 3.11) against the Mouse-RNAseq reference dataset, available at https://rdrr.io/github/dviraran/SingleR/man/mouse.rnaseq.html. Cell-type marker lists were generated using Seurat's native FindMarkers function with a log fold change threshold of 0.25 using the ROC test to assign predictive power to each gene. The 200 most highly ranked genes from this analysis for each cluster were used to perform gene set enrichment analysis in a programmatic fashion using EnrichR (version 2.1). (Chen et al., 2013)

The scRNA sequencing data generated during this study has been sent to the NCBI's Gene expression Omnibus for deposition and will be accessible on request.

### 17. Pseudotime Analysis:

Pseudotime analysis was performed using the Monocle 3 package in R (version 3 0.2.0). (Trapnell et al., 2014) Counts for individual cells were preprocessed using PCA with 15 dimensions following log-normalization. Dimensional reduction was performed using a UMAP reduction with min_dist=0.5, n_neighbors=30, and repulsion.strength=2.0. Cells were then clustered using Monocle 3's Louvain implementation with a resolution of 1e-5. A principal graph was then learned from the reduced dimension space using reversed graph embedding with default parameters, and cell order selection was made from the two elements at either end of the trajectory. Pseudotime trajectory heatmaps were created using the Monocle 2 package in R.

### 18. CytoTRACE Analysis:

We utilized the recently developed bioinformatics tool CytoTRACE to compare differentiation states among cells in our dataset (https://cytotrace.stanford.edu/). (Gulati et al., 2020) This tool analyzes the number of uniquely expressed genes per cell, as well as other factors like distribution of mRNA content and number of RNA copies per gene, to calculate a score assessing the differentiation and developmental potential of each cell (lowest differentiation and highest developmental potential at 1; highest differentiation and lowest developmental potential at 0). Cells are then ordered by their predicted differentiation status. CytoTRACE analysis was performed using default parameters for each fibroblast in our dataset.

### 19. GeneTrail Analysis:

Using GeneTrail 3, an over-representation analysis (ORA) was performed for each cell using the 500 most expressed protein-coding genes on gene sets from Gene Ontology. (Gerstner et al., 2020) P values were adjusted using the Benjamini-Hochberg procedure, and gene sets were required to have between 2 and 1,000 genes. Stacked violin plots were generated using the Scanpy package. (Wolf et al., 2018)

### 20. CellChat Receptor-Ligand Analysis:

To evaluate the potential for interactions between different cell types in our dataset, we applied the recently developed CellChat platform. (Jin et al., 2021) This was implemented using our scRNA-seq Seurat object in R, in conjunction with the standalone CellChat Shiny App for its Cell-Cell Communication Atlas Explorer. Cells were binned according to the SingleR-defined cell type classifications, with fibroblast cells subsetted based on their location within either the scarring or regenerative pseudotime arms. Default parameterizations were used throughout, and Secreted Signaling, ECM-Receptor, and Cell-Cell Contact relationships were considered.

### 21. RNA Velocity Analysis:

RNA velocity analysis was performed using the scVelo package. (Bergen et al., 2020a) scVelo uses a likelihood-based dynamical model to solve the full transcriptional dynamics of spliced and unspliced mRNA kinetics of each gene. RNA velocity analysis allowed us to identify transient cellular states in our dataset and to predict the directional progression of transcriptomic signatures along the identified trajectories. These predictions are based on gene-specific rates of transcription, splicing, and degradation of mRNA to estimate each cell's position in their own underlying differentiation process. The RNA velocity across all genes is then projected as a stream of arrows on the UMAP embedding.

### 22. CODEX Spatial Analysis:

To spatially phenotype the pig specimens, we used Co-Detection by Indexing (CODEX), a novel assay in which markers are labeled with oligonucleotide-conjugated antibodies and iteratively imaged between cyclic additions and washouts of dye-labeled oligonucleotides. A custom CODEX panel was designed to assess wound cells within the tissue see Table 1). In brief, primary antibodies were individually barcoded and validated using the commercial supplier's protocols. OCT for mouse or paraffin blocks for human xenograft (n=3 per group) were sectioned at 8 μm thickness onto coverslips for CODEX antibody staining. Antigens were retrieved by standard citrate-EDTA processing prior to addition of CODEX antibodies. Using a CODEX-integrated Keyence BZ-X instrument (Akoya Biosciences) image acquisition was then performed. Using software from Akoya Biosciences the raw images were process, with cell segmentation, and rendering.

The CODEX was visualized using Akoya Biosciences Multiplex Analysis Viewer (MAV) in ImageJ. The resulting .fcs files were then concatenated in FlowJo and imported into the Monocle3 and STvEA R packages for further analysis. After debris removal, the processed UMAP manifold was analyzed through Monocle3 with a post-manifold threshold of >10,000 cells per cluster. Analysis of the protein staining patterns was then used to assign cell types. The cell interactions were then inferred using STvEA for cell types at >2.5% of total abundance at k=20 nearest neighbors to quantify cell spatial interactions, and differential interaction maps were generated using graph scores.

### 23. Visium Spatial Transcriptomic Analysis:

Wound specimens were rapidly harvested and flash frozen in OCT. Using the Visium Tissue Optimization Slide and Reagent Kit, permeabilization time was optimized at a thickness at 10 μm per section and 37 minutes for mouse tissue. Following cryo-sectioning at −20 degrees onto gene expression slides the expression slide and reagent kit was used to produce sequencing libraries. The libraries were then sequences using NextSeq (Illumina). Following demultiplication the raw FASTQ files and histology images were processed by sample with the Space Ranger software for genome alignment. The raw spaceranger output files for each sample was then read into a Seurat class object in R using Seurat's Load10× function. Data was normalized using the SCT transform with default parameters. To ascertain the integration of our scRNAseq and Visium spatial analysis we employed the FindTransferAnchors( ) function from Seurat, which allowed the alignment of data using the two datasets. This cross-platform linkage is performed serially in an unconstrained and constrained fashion.

### 24. Statistical Methods:

Statistical testing was performed in GraphPad Prism v9 unless otherwise stated. For two-group comparisons, unpaired t-tests were used. For multi-group analysis, one-way ANOVAs were used with Tukey's post hoc corrections to compare groups; p<0.05 conferred statistical significance for all tests.

### D. RESULTS

1. Adipocytes transition to ECM-producing fibroblasts during wound repair and contribute to scarring

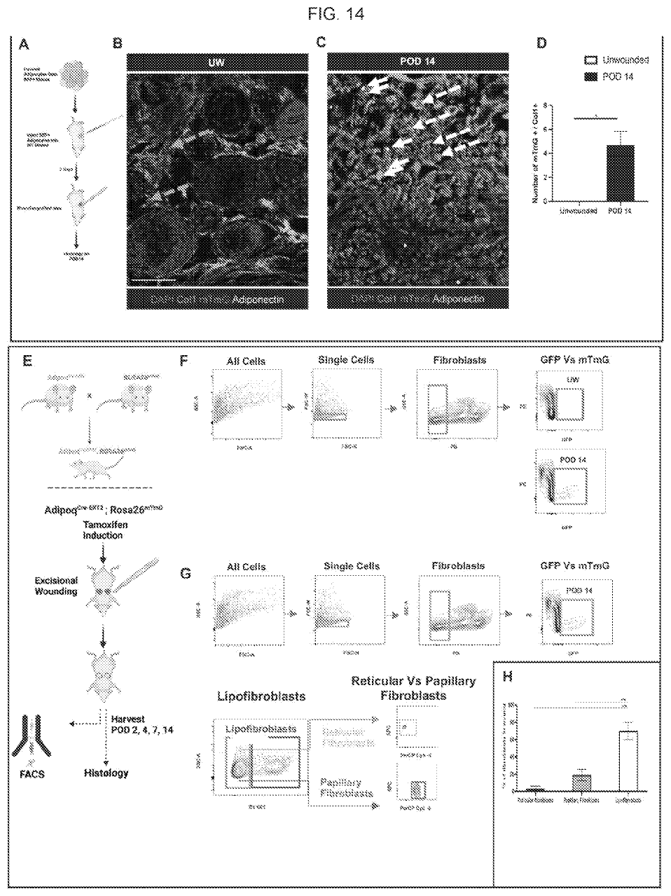

To trace adipocyte fate in wounds, we first developed an adipocyte transplantation and wounding model (FIG. 14A). We transplanted Tomato⁺adipocytes from R26^{mTmG} mice into the dorsal dermis of non-fluorescent wildtype recipient mice, then wounded within the cell-engrafted region. Wounds were stented using silicone rings to prevent rapid contraction, (Mascharak et al., 2021a) and excised skin was saved as an unwounded cell-engrafted control. In unwounded skin, few Tomato⁺ cells were observed (FIG. 14B, dotted orange arrows). In contrast, in wounds harvested at postoperative day (POD) 14 (the time of complete wound re-epithelialization), a greater number of Tomato⁺ cells were observed; interestingly, these cells had phenotypic properties of both adipocyte and fibroblast identity, expressing adiponectin (Adipoq; mature adipocyte marker) and type 1 collagen (Col1; not substantially expressed by adipocytes at homeostasis) (FIG. 14C, solid white arrows), suggesting that these cells may be in the process of transitioning from adipocytes to fibroblasts. We also observed Tomato⁺ cells that were Adipoq⁻/Col1⁺ (FIG. 14C, dotted white arrows) and a significant increase in the number of transplanted (Tomato⁺) cells expressing Col1 by POD 14 (FIG. 14D), consistent with adipocytes having differentiated into fibroblasts in healing wounds.

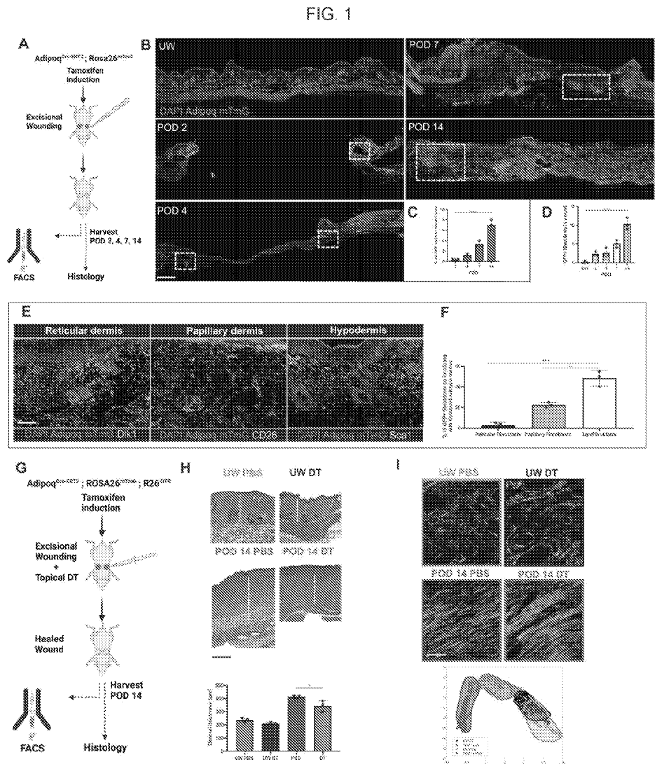

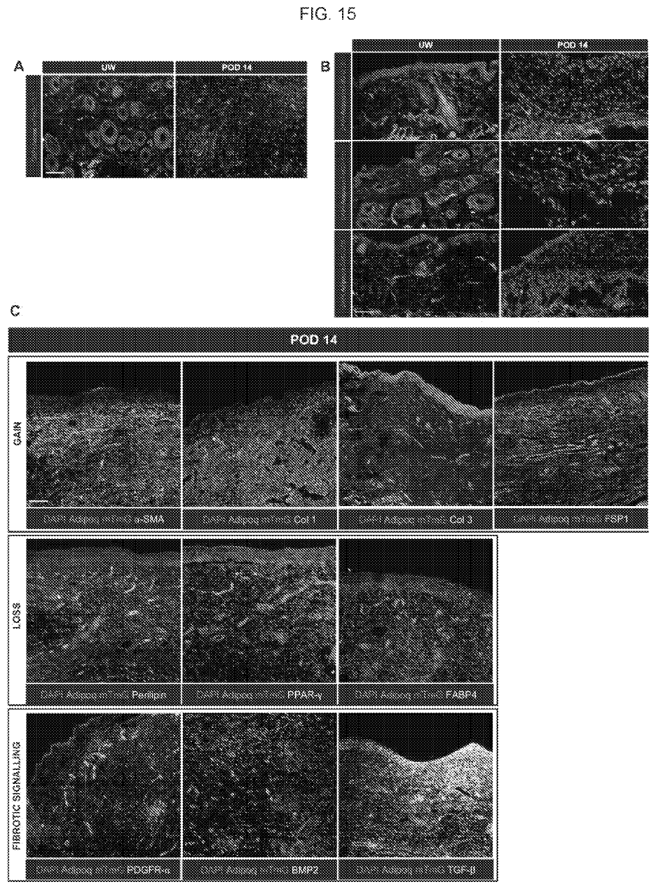

While these results strongly suggested that adipocytes in wounds transitioned to fibroblasts, it was possible that a small number of contaminating fibroblasts could have been inadvertently engrafted and then proliferated within wounds to produce the observed Tomato⁺ fibroblasts. To address this potential concern, we generated, tamoxifen-induced, and wounded Adipoq^{Cre-ERT}; R26^{mTmG} mice (FIGS. 1A and 14E). In this model, mature adipocytes (Adipoq⁺) and their progeny express green fluorescent protein (GFP), allowing robust identification of wound fibroblasts that arose from adipocytes (GFP⁺); all other cells express Tomato. On histology, GFP expression in uninjured skin was confined to large, round subcutaneous cells consistent with mature adipocytes; over the course of wound healing, GFP⁺ cells increased in prevalence throughout the dermis (FIG. 1B-C). Fluorescence-activated cell sorting (FACS; based on lineage depletion as previously published (Leavitt et al., 2017)) showed that GFP⁺ (adipocyte-derived) fibroblasts were rare in unwounded skin but increased in wounds over time, with adipocyte-derived fibroblasts (ADFs) comprising ˜10% of wound fibroblasts by POD 14 (FIGS. 1D and 14F). Further supporting fibroblast identity, GFP⁺ cells in POD 14 wounds did not express endothelial or immune markers (FIG. 15A-B), largely failed to express adipocyte markers (e.g., perilipin), and instead expressed typical scar fibroblast markers (e.g., collagens, alpha-smooth muscle actin [αSMA]; FIG. 15C, top and middle rows). These ADFs also colocalized with known fibroblast signaling factors such as transforming growth factor beta (TGFβ) (FIG. 15C, bottom row). Collectively, these results were consistent with ADFs losing their adipocyte identity over the course of wound healing and transitioning to activated, ECM-producing fibroblasts.

Unwounded skin contains multiple anatomically distinct fibroblast populations with unique surface markers, which we and others have implicated in differing wound phenotypes and fibrogenic properties. (Driskell et al., 2013; Mascharak et al., 2021a) We used histology and multi-color FACS (FIG. 14G) profiling of unwounded skin and wounds in Adipoq^{Cre-ERT}; R26^{mTmG} mice to separately identify fibroblasts of the papillary dermis (CD26⁺Sca1⁻), reticular dermis (Dlk1⁺Sca1⁻), and hypodermis (lipofibroblasts; Dlk1^{+/-}Sca1⁺). The majority of ADFs were Sca1⁺ (lipofibroblasts); a smaller number expressed papillary and reticular dermal fibroblast markers (FIGS. 1E-F and 14G-H).

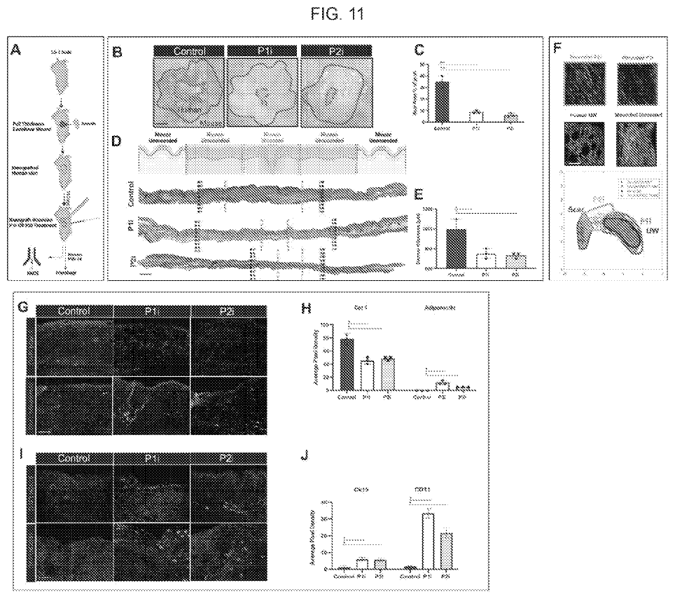

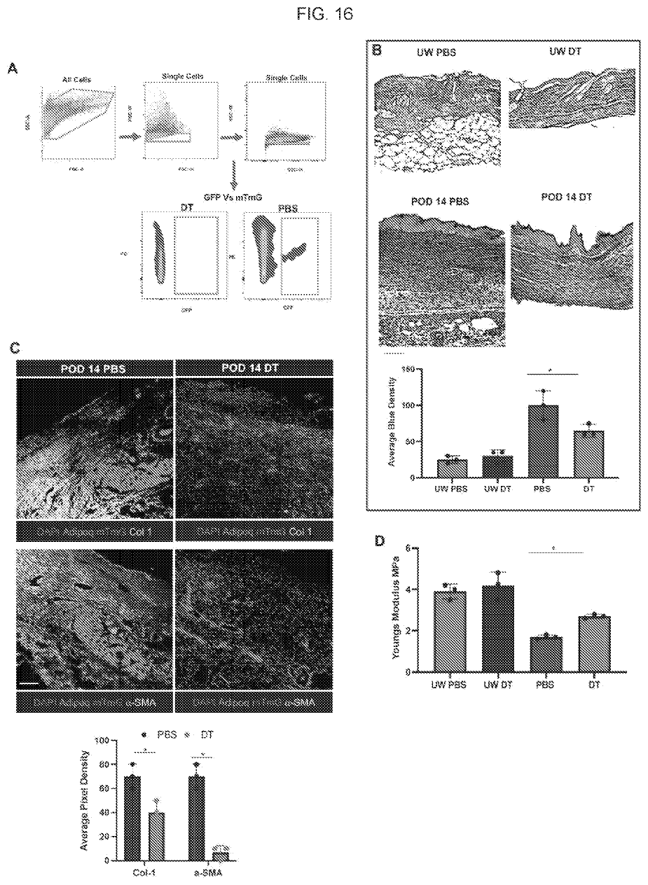

We next developed Adipoq^{Cre-ERT}; R26^{mTmG}; R26^{iDTR} mice to enable selective ablation of mature adipocytes following tamoxifen induction (FIG. 1G). Confirming efficacy of ablation, unwounded skin and POD 14 wounds treated with phosphate-buffered saline (PBS) vehicle control contained abundant subdermal adipocytes and GFP⁺ ADFs, while those treated with diphtheria toxin (DT) had minimal to no subdermal fat and no GFP⁺ cells in wounds (FIG. 1H, top panels, and FIG. 16A). Further, DT-treated scars had significantly reduced dermal thickness (FIG. 1H, bottom panel), less dense extracellular matrix (ECM), and decreased Col1 and α-SMA content compared to PBS control scars (FIG. 16B-C). Quantitative analysis of ECM ultrastructure (Mascharak et al., 2021a) revealed that ECM of DT-treated wounds was less scar-like and more closely resembled unwounded skin, compared to the distinct scar ECM structure of control (PBS) wounds (FIG. 11). Scars, despite being more densely collagenous than skin, are mechanically inferior, only ever regaining up to 80% of the strength of unwounded skin; (Mascharak et al., 2021b) adipocyte-ablated wounds had mechanical properties intermediate between those of unwounded skin and control scars (FIG. 16D).

### 2. Adipocyte-Derived Fibroblasts Comprise Distinct “Mechanically Naïve” and “Mechanically Activated” Subpopulations

While complete adipocyte ablation did reduce scarring, this effect fell short of complete regeneration. Although our results supported a role for adipocytes in driving fibrosis, prior studies and clinical correlates suggest that adipocytes may mitigate fibrosis in certain settings; (Almadori et al., 2019) we thus wondered whether there may exist multiple dermal adipocyte subtypes with distinct functional roles (i.e., some pro-scarring, some pro-regenerative). If this were the case, a more optimal strategy may be to specifically target only those pro-scarring adipocytes, rather than blanket ablation of all adipocytes. We hypothesized that a subset of adipocytes may respond to wound-specific cues to adopt a pro-fibrotic fibroblast phenotype, and sought to robustly characterize the dynamics and drivers of adipocyte-to-fibroblast transition.

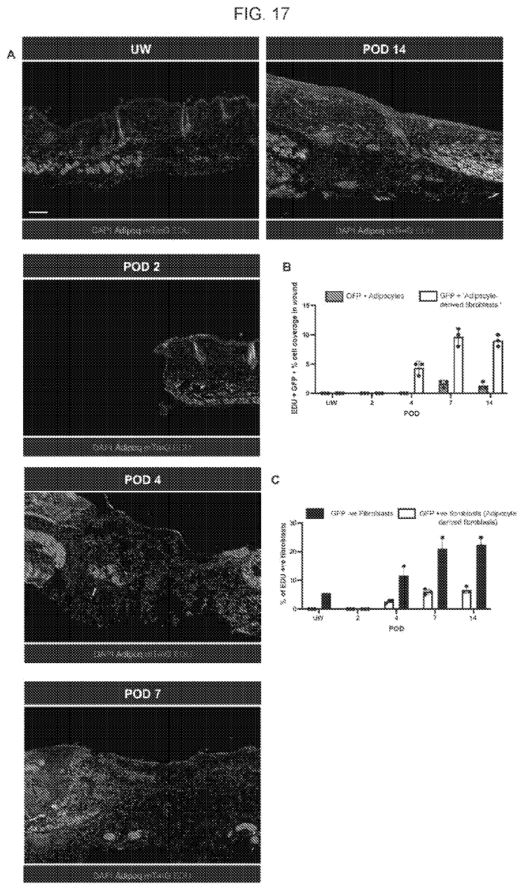

We first examined the cellular proliferation dynamics of adipocytes' contributions to scarring. EdU analysis revealed that adipocytes were minimally proliferative in both wounds and unwounded skin, whereas EdU incorporation appeared in ADFs by POD 4 and increased by POD 7 and 14 (FIG. 17A-C), suggesting an initial adipocyte-to-fibroblast transition followed by proliferation of ADFs post-transition. We next used R26^{VT2/GK3} (“Rainbow”) reporter mice to study adipocyte clonal dynamics in wounds. In these mice, all cells initially express GFP; Cre induces stochastic R26^{VT2/GK3} cassette recombination, resulting in random expression of one of three distinct fluorophores (mCerulean [blue], mOrange [orange], or mCherry [red]). Following recombination, that cell and its progeny will express the same color, allowing histologic identification of clonal expansion (clusters of multiple, identically colored cells). We performed tamoxifen induction and splinted excisional wounding of Adipoq^{Cre-ERT}; R26^{VT2/GK3} mice (FIG. 2A). POD 14 Adipoq^{Cre-ERT}; R26^{VT2/GK3} wounds contained numerous ADFs (mOrange⁺, mCerulean⁺, or mCherry⁺ cells that colocalized with Col1) and clusters of same-colored ADFs (FIG. 2B-C). Collectively, these results supported that individual adipocytes give rise to a larger number of fibroblasts via polyclonal expansion, suggesting that a subset of adipocytes may possibly serve as pro-fibrotic fibroblast progenitors in the injury setting.

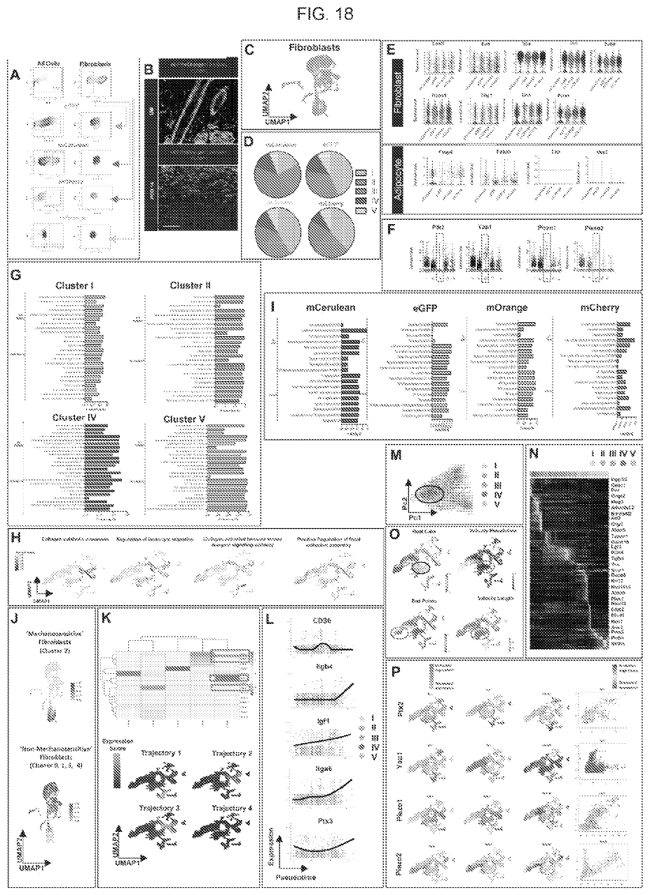

Next, to interrogate ADF heterogeneity and molecular signaling, we FACS-isolated fibroblasts expressing each Rainbow color, then subjected each population to single-cell RNA-sequencing (scRNA-seq) (FIGS. 2D and 18A). Rainbow construct specificity was confirmed by the absence of reporter recombination without tamoxifen induction (FIG. 18B). Louvain-based (Seurat) clustering identified five transcriptionally distinct fibroblast clusters (denoted with Roman numerals I-V; FIGS. 2E-F and 18C); notably, one of these clusters (cluster III) comprised almost exclusively adipocyte-derived fibroblasts (FIGS. 2E, bottom, and 18D), suggesting that this cluster identity was unique to cells of adipocyte origin. Transcriptomic analysis revealed that fibroblasts, regardless of clone color, expressed negligible adipocyte genes and instead expressed typical wound fibroblast genes (FIGS. 2G and 18E). We and others have previously implicated mechanical signaling (e.g., canonical focal adhesion kinase [FAK]/Yes-associated protein [YAP]-mediated mechanotransduction) as modulating wound fibrosis; (Chen et al., 2021b; Mascharak et al., 2021a; Mascharak et al., 2021b; Mascharak et al., 2020) interestingly, scRNA-seq revealed enrichment for FAK (Ptk2) and YAP (Yap1), as well as the mechanosensitive ion channel components Piezo1 and Piezo2, (He et al., 2018; Holt et al., 2021; Jin et al., 2020; Pethö et al., 2019) in all fibroblast clusters except cluster III (the cluster containing almost entirely adipocyte-derived fibroblasts; FIGS. 2H and 18F). Further interrogation of cluster III using Gene Ontology (GO) pathway analysis revealed enrichment for non-traditional fibroblast pathways including lipoprotein particle receptor-related processes, thermogenesis, and adipogenesis (FIG. 2I). In stark contrast, all other fibroblast clusters were enriched for numerous “typical” fibrosis-related pathways, including mechanical activation terms (e.g., “focal adhesion,” “cell-matrix adhesion mediator activity”) and fibrosis-related terms (e.g., “collagen-containing extracellular matrix”; FIG. 18G). GeneTrail analysis further highlighted that Seurat fibroblast clusters I, II, IV, and V showed comparative upregulation of collagen processes, collagen-activated, focal adhesion assembly, and leukocyte migration (FIG. 18H). Enrichment for typical fibrosis GO terms was observed across all Rainbow clone colors (FIG. 18I). One population in particular (cluster II) exhibited a transcriptional profile consistent with a mechanosensitive fibroblast subpopulation previously described by our group, which clonally proliferates toward the wound center following skin injury (Foster et al., 2021) (FIG. 18J).

We also analyzed cells using CytoTRACE (a computational method for predicting cell differentiation states based on relative transcriptional diversity (Gulati et al., 2020)), which revealed that cluster Ill represented a dramatically distinct differentiation state from all other fibroblast clusters (FIG. 2J). Pseudotime analysis confirmed the presence of clear differentiation trajectories originating in cluster III cells and progressing outward to all other clusters (FIGS. 2K and 18K); differentiation along these trajectories was associated with increased expression of known mechanical signaling genes and pro-fibrotic markers (FIGS. 2L and 18L). We then applied RNA velocity analysis (scVelo), which compares expression of unspliced pre-mRNA and mature spliced mRNA to infer directional information for transcriptional dynamics within the dermis. (Bergen et al., 2020b) This approach again predicted differentiation trajectories originating from cluster III cells (FIG. 18M-P). RNA velocity analysis using root cell prediction and velocity length supported that this differentiation trajectory starts from cluster III (FIG. 18O). Collectively, these results led us to conclude that cluster III may represent an intermediate differentiation state, in which adipocytes have transitioned to a fibroblast fate but remain “mechanically naïve” and may retain some hallmarks of adipocyte transcriptional identity; continued wound mechanical stimuli could then lead to further differentiation to a fully mechanically sensitive/activated and pro-fibrotic scar fibroblast state (FIG. 2M).

### 3. Mechanical Forces are Sufficient to Drive Adipocyte-to-Fibroblast Conversion in a Process Involving Piezo1 and Piezo1 Signaling

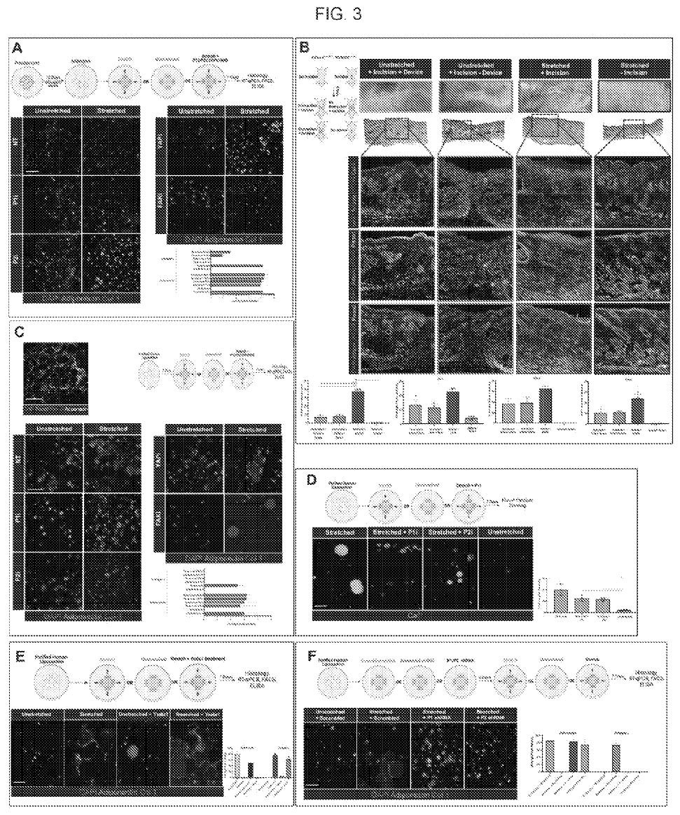

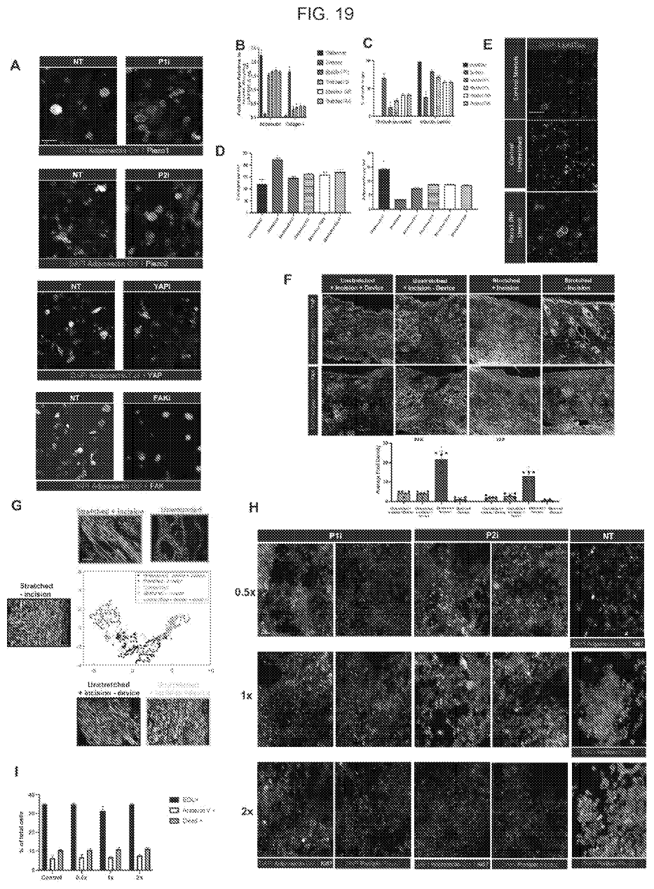

We were interested to find transcriptional evidence for the relevance of mechanosignaling in ADFs. It is well known that tissue mechanical forces activate fibroblasts to drive scarring. (Mascharak et al., 2021b) However, while in vitro evidence suggests that adipocytes alter phenotype in response to mechanical environment, (Hossain et al., 2010; Yuan et al., 2015) whether mechanics modulate differentiation of wound adipocytes remains unknown. We sought to determine whether mechanical signaling could serve as a “switch” to guide adipocytes toward a fibrogenic (in response to mechanical strain) versus adipogenic (without strain or with blocked mechanotransduction) fate. As traditional floating adipocyte culture does not allow alterations of mechanical environment and fails to replicate cells' native 3D environment, we adapted a published 3D culture system (Chen et al., 2021a) in which cell-seeded hydrogels undergo controlled stretching, permitting precise mechanomodulation, for mouse adipocytes (FIG. 3A, top panel). In the absence of applied stretch, mouse adipocytes had widespread expression of adiponectin and absent fibroblast markers; remarkably, when stretch was applied, a large proportion of adipocytes adopted a fibrogenic phenotype, losing adiponectin and instead expressing Col1 (FIG. 3A, bottom panels). When small molecule inhibitors were used to block key mechanosignaling genes (Piezo1 [P1i], Piezo2 [P2i], FAK [FAKi], or YAP [YAPi]; FIG. 19A), the stretch-induced phenotypic switch was abrogated (FIGS. 3A and 19B-D). Collectively, these findings suggested that mechanical stress, communicated at the cellular level via mechanosensation pathways including Piezo1 and Piezo2, was sufficient to drive adipocyte-to-fibroblast conversion.

It was next critical to confirm these findings in vivo. We applied a model that subjects mouse incisional wounds to increased tension, producing scars that resemble human hypertrophic scars (HTS), (Aarabi et al., 2007) in Adipoq^{Cre-ERT}; R26^{VT2/GK3} mice (FIG. 3B, top left) to directly determine whether mechanical loading promotes adipocyte-to-fibroblast conversion in wounds in vivo. When wounds were mechanically stretched, scars were more fibrotic and had substantially increased ADFs, Col1 and mechanical signaling factors including Piezo1 and Piezo2 (FIGS. 3B and 19F-G).

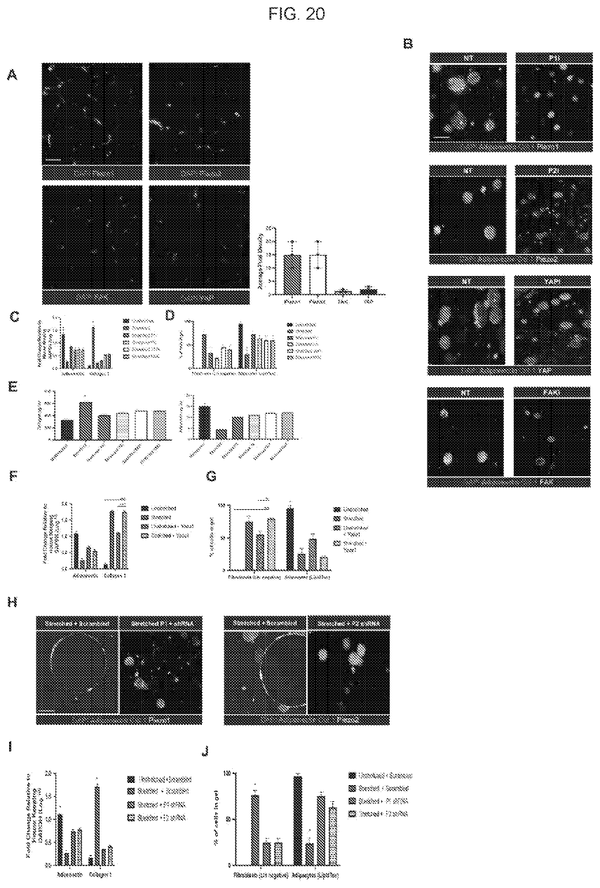

Given that significant differences may exist between mouse and human adipocytes, we sought to validate our mouse findings in human cells. IF staining revealed that Piezo1 and Piezo2 were expressed in human adipocytes while, interestingly, FAK and YAP were minimally expressed (FIGS. 3C, top left, and 20A). We applied our mechanomodulatory culture system to human adipocytes (FIG. 3C, top right) and found that, similar to mouse adipocytes, stretching caused these cells to express Col1 and lose adipocyte markers; this transition was again abrogated by inhibiting mechanosignaling (FIGS. 3C, bottom, and 20B-E). Interestingly, stretching also induced substantial hypertrophy of human adipocytes (previously linked with pathological remodeling/fibrosis of adipose tissue (Vishvanath and Gupta, 2019)), which was reversed with P1i and P2i but not FAKi or YAPi (FIG. 3C, bottom). Consistent with stretching driving activation of calcium-permeable Piezo mechanosensitive ion channels, (Matsunaga et al., 2021; Romac et al., 2018) we observed increased calcium staining with stretching that decreased with P1i or P2i (FIG. 3D). Our inhibitor experiments suggested that mechanosignaling was necessary for adipocyte-fibroblast transition in response to stretch; conversely, applying Yoda1 (a Piezo1 agonist (Syeda et al., 2015)) was sufficient to drive adipocyte hypertrophy and conversion to fibroblasts even in the absence of applied stretch (FIGS. 3E and 20F-G). Given possible off-target effects of small molecules, we also used short hairpin RNA (shRNA) to knockdown Piezo1 and Piezo2 (FIGS. 3F, top, and 20H), which blocked stretch-induced adipocyte hypertrophy and adipocyte-to-fibroblast transition (FIGS. 3F and 20H-J).

### 4. Blocking Piezo Mechanosignaling in Adipocytes Reduces Scarring and Fibrosis

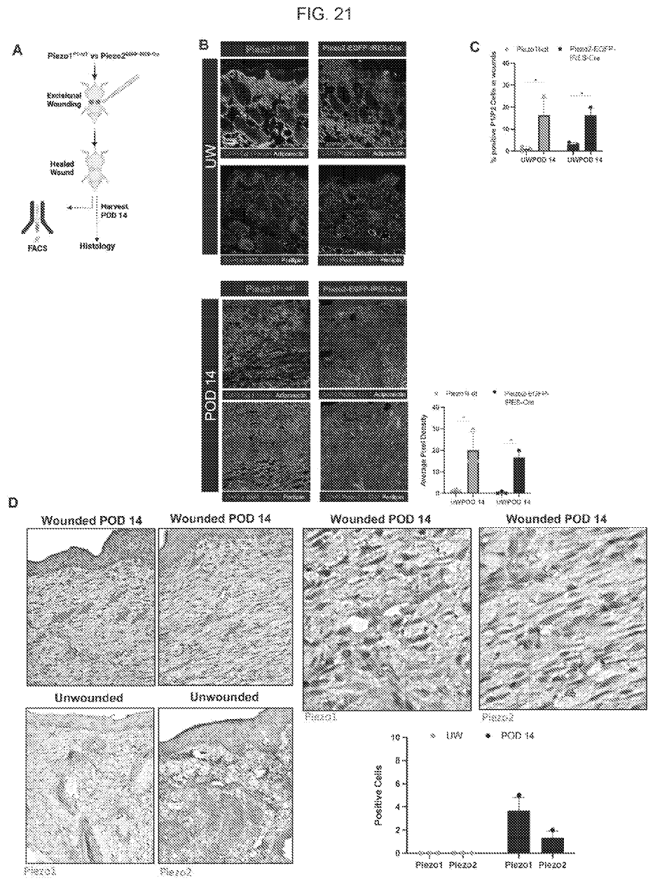

Given the dramatic effects of Piezo1 and Piezo2 inhibition on adipocyte differentiation into fibroblasts in vitro, we next examined whether blocking adipocyte mechanosensing in vivo could prevent adipocyte-to-fibroblast transition in wounds and yield reduced scarring. We focused on Piezo1 and Piezo2 because, compared to FAK or YAP, their expression was more adipocyte-specific (FIG. 20A) and their inhibition reversed both hypertrophy and gene expression changes in stretched adipocytes, compared to gene expression only with FAK/YAP (FIG. 3C). Further, genetic lineage tracing and in situ hybridization confirmed that Piezo1 and Piezo2 expressing cells were present and lost adipocyte/gained fibroblast markers in wounds (FIG. 21A-D).

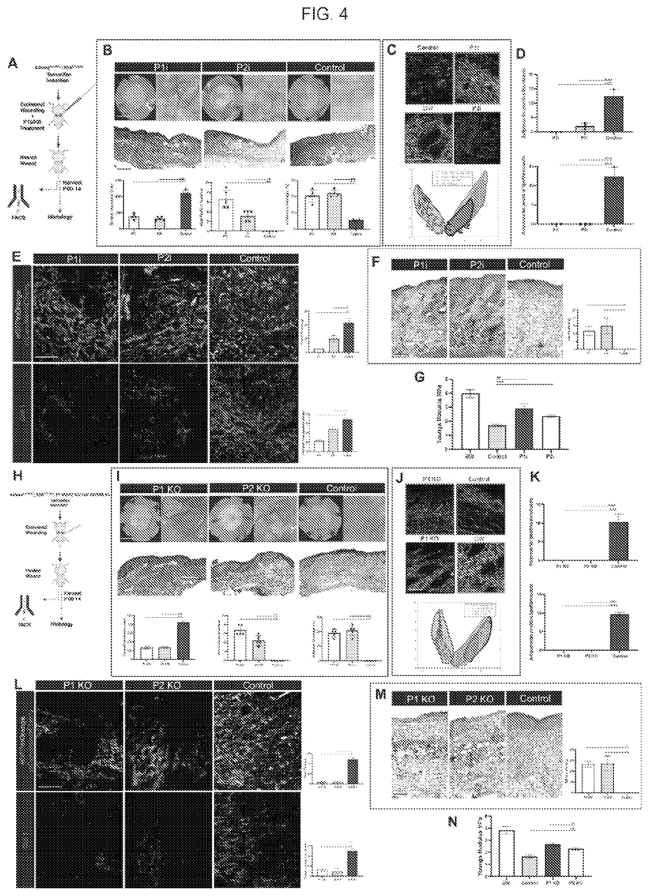

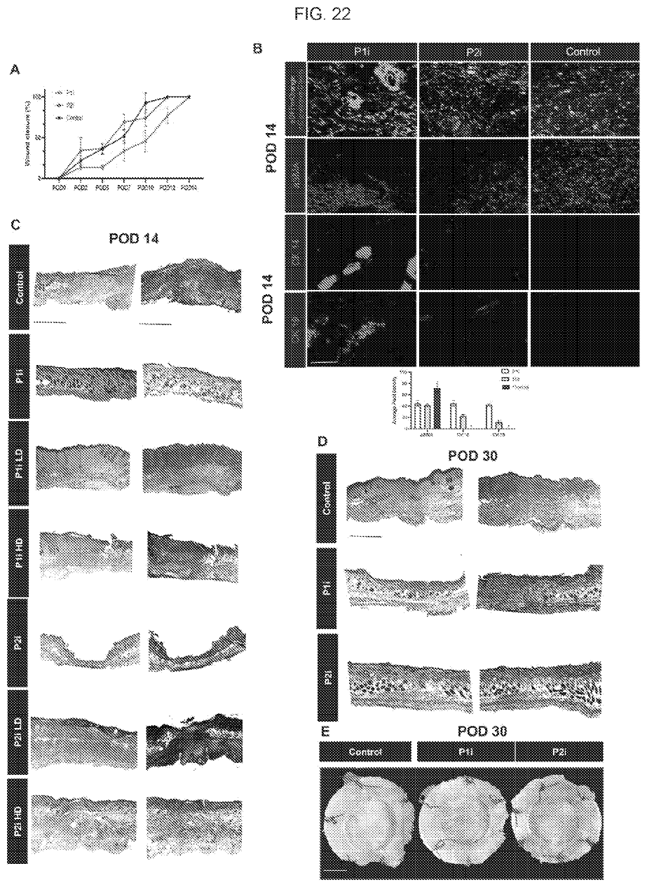

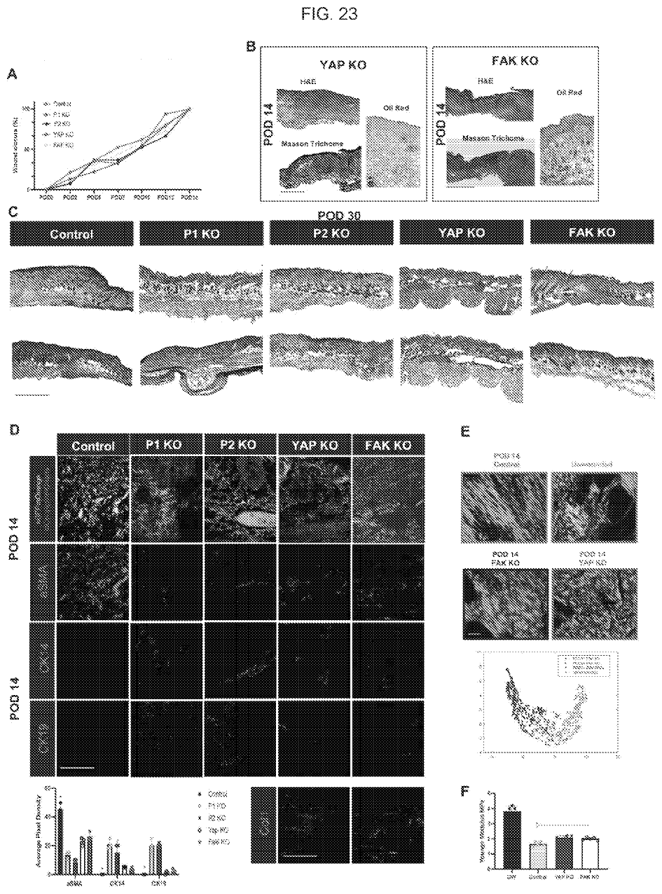

We treated wounds in tamoxifen-induced Adipoq^{Cre-ERT}; R26^{VT2/GK3} mice with P1i or P2i or PBS (vehicle control) via local injection into the wound edges at POD 0 (FIG. 4A). Doses were optimized based on in vitro dosing and confirmed with toxicity testing (FIGS. 19H-I). P1i and P2i did not significantly affect time to complete wound re-epithelialization (FIG. 22A). Grossly and histologically, Piezo inhibitor-treated wounds yielded less-apparent scars, with significantly decreased scar thickness; increased adipocytes; and (particularly in P1i wounds) numerous structures morphologically consistent with unerupted hair follicles (HF; FIG. 4B). While control wounds had ECM distinct from unwounded skin's (consistent with fibrotic scars), Piezo-inhibited wounds had ECM features overlapping unwounded skin's (FIG. 4C), suggesting that mechanotransduction inhibition led to regeneration of normal skin-like ECM. ADFs were significantly reduced in P2i and absent in P1i wounds (FIGS. 4D-E and 22B, top), suggesting that Piezo inhibition prevented adipocyte-to-fibroblast conversion. P1i and P2i wounds had reduced Col1 and α-SMA and less dense ECM at both POD 14 and POD 30 (early remodeling phase of wound repair), while Oil Red O staining and IF for cytokeratins (CK) 14/19 (markers of regenerating appendages) demonstrated presence of putative regenerating sebaceous glands/HF (FIGS. 4E-F and 22B-D). Congruent with our previous study (Mascharak et al., 2021a), where we found YAPi inhibition to allow for hair regrowth at POD 30, P1i/P2i allowed for hair follicle formation at POD 30 (FIG. 22E). Finally, mechanical testing confirmed that P1i (and, to a lesser extent, P2i) reverted scars to more unwounded-like mechanical properties (FIG. 4G). While these results suggested that P1i or P2i was sufficient to prevent adipocyte-to-fibroblast conversion and reduce wound fibrosis, small molecules may have off-target effects. Thus, we developed Adipoq^{Cre-ERT}; R26^{VT2/GK3}; Piezo1^{fl/+} (Piezo1^{fl/+}) and Adipoq^{Cre-ERT}; R26^{VT2/GK3}; Piezo2^{fl/+} (Piezo2^{fl/+}) mice and administered tamoxifen to induce both Rainbow reporter combination and Piezo1 or Piezo2 deletion in adipocytes prior to splinted excisional wounding; we similarly generated adipocyte-targeted YAP and FAK KO mice to compare the efficacy of ablating these canonical mechanotransduction molecules in adipocytes (FIG. 4H). None of these genes' (P1, P2, YAP, FAK) deletion in adipocytes significantly affected time to wound closure (FIG. 23A). Similar to the effects of P1i and P2i, Piezo1 and Piezo2 KO yielded significantly reduced scarring with fat accumulation and regeneration of dermal appendages in wounds (FIG. 4I-M). Notably, tensile testing revealed that Piezo KO wounds had mechanical properties similar to those of unwounded skin rather than scars (FIG. 4N). Supporting our in vitro evidence suggesting that YAP and FAK inhibition were less effective at preventing mechanically-activated adipocyte changes compared to Piezo inhibition, fibrosis was mitigated to a much lesser extent in adipocyte-targeted YAP and FAK KO wounds (FIG. 23B-F). Collectively, these results demonstrated that adipocyte-specific Piezo1 or Piezo2 blockade prevented adipocyte-to-fibroblast conversion and had anti-scarring/pro-regenerative effects in wound healing.

### 5. Piezo Blockade Prevents Activation of “Mechanically Naïve” to “Mechanically Activated” Adipocyte-Derived Fibroblasts

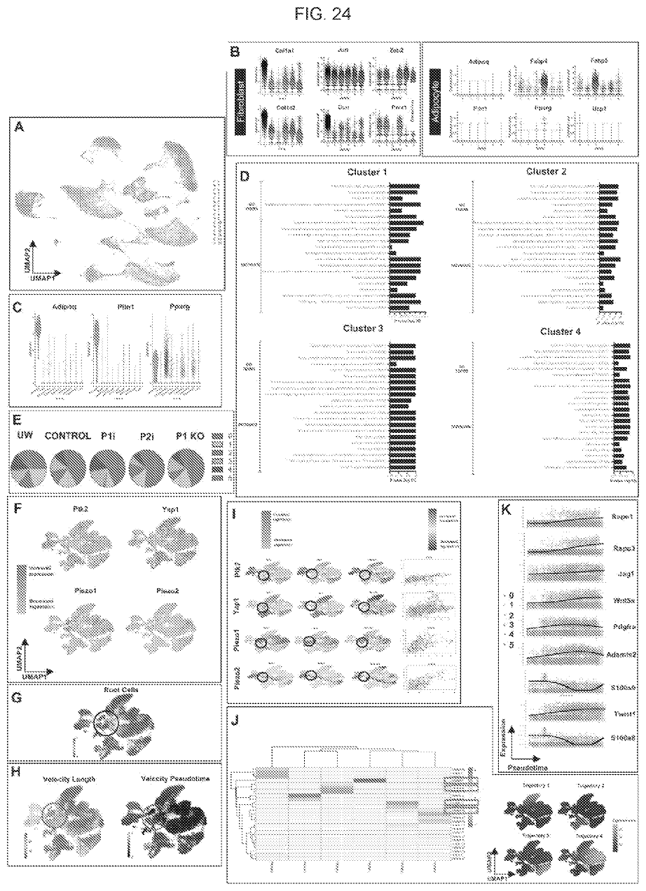

In order to more deeply interrogate the mechanism(s) by which Piezo inhibition prevented scarring and induced wound regeneration, we performed scRNA-seq of cells from five wound healing conditions (all wounds at POD 14): wounds treated with small molecule Piezo inhibitors (P1i or P2i); wounds in Piezo1 KO mice; control wounds (wildtype mice without treatment); or unwounded skin (FIG. 5A). Sequenced cells included all expected wound cell types, including fibroblasts, keratinocytes, endothelial cells, inflammatory cells, and a small number of adipocytes (FIGS. 5B and 24A-C). Following sequencing, we performed in silico selection for fibroblasts (based on individual sequenced cell transcriptomic profiles; FIG. 5B); we confirmed that these cells expressed canonical fibroblast genes and had minimal expression of adipocyte identity genes (FIG. 24B). Six transcriptionally distinct fibroblast clusters (designated clusters 0-5) were identified by Louvain-based clustering (Seurat; FIG. 5C-D). To determine how these populations related to those identified by our original scRNA-seq fibroblast dataset (FIG. 2E), we applied an anchor-based label transform approach to project the current six clusters (FIG. 5C) onto the original five scRNA-seq clusters (FIG. 2E) in a K-nearest neighbor-based fashion (FIG. 5E, top). (Stuart et al., 2019) These projections supported strong similarities between each cluster from the original dataset and specific clusters in the current dataset, most notably between our previous cluster III (the “mechanically naïve” subpopulation from FIG. 2) and new cluster 5 (FIG. 5E). Supporting that the newly-identified cluster 5 shared the unique transcriptional program previously identified as the “mechanically naïve” fibroblast cluster in FIG. 2, GO pathway analysis of cluster 5 revealed terms related to lipoproteins, visceral fat, and lipid functions; in contrast, all other clusters were enriched for typical pro-fibrotic fibroblast terms such as focal adhesion and ECM-related terms as well as mechanical signaling genes (FIGS. 5F and 24D). Notably, cluster 5 was less represented in control (scarring) wounds and more highly represented in Piezo inhibition and KO wound cells as well as unwounded skin (FIGS. 5G and 24E); P1i wounds in particular also contained relatively more adipocytes and fewer fibroblasts (FIG. 5H). Cluster 5 was distinguished by lower expression of key mechanical signaling genes (FIG. 24F), consistent with its putatively “mechanically naïve” identity. Using RNA velocity analysis, we again identified a differentiation trajectory originating from mechanically naïve cells (cluster 5; FIGS. 51 and 24G-I) which we then used as a starting point for pseudotime analysis (FIGS. 5J and 24J-K). Both analyses further supported cellular transition from “mechanically naïve” fibroblasts and more traditional fibroblast subpopulations (FIG. 24G-I). Overall, these results were again consistent with the presence of two distinct differentiation states of adipocyte-derived fibroblasts: a mechanically naïve population (cluster 5), which is enriched in states of inhibited adipocyte mechanotransduction; and a more mechanically primed population, which is relatively enriched in typical scarring wound conditions and is more fibrotic (FIG. 5K).

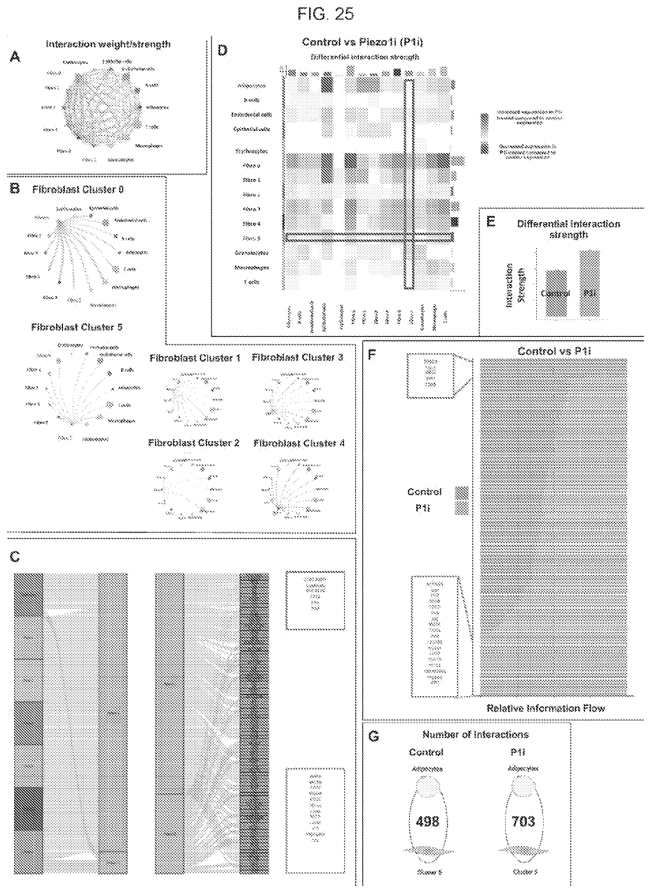

Finally, we analyzed our scRNA-seq data using CellChat, a computational method for inferring cell-cell interactions (Jin et al., 2021) that we previously applied to study dynamics of wound fibrosis versus regeneration. (Mascharak et al., 2020) Examining all cell-cell interactions revealed fewer overall interactions with cluster 5 (“mechanically naïve”) fibroblasts compared to other fibroblast clusters (especially cluster 0) (FIG. 25A-B). Extensive cell signaling was found between fibroblasts and adipocytes, including a unique pattern of signaling between cluster 5 fibroblasts and adipocytes that was not present with other fibroblast clusters (FIG. 25C). Following P1i treatment, we identified an increase in signaling between cluster 5 fibroblasts and most other cell types, compared to decreased signaling of other fibroblast clusters (FIG. 25D-E). P1i treatment also enhanced signaling of known adipocyte pathways including Leptin (Lep), (Harris, 2014) Chemerin, (Goralski et al., 2007) and PD-L1 (Ingram et al., 2017) (FIG. 25F). Lastly, P1i treatment enhanced interactions between cluster 5 fibroblasts and adipocytes (FIG. 25G), suggesting that Piezo inhibition alters crosstalk between adipocytes and fibroblasts upon wounding.

### 6. Piezo Inhibition Reduces Fibrosis in Existing Mouse Scars

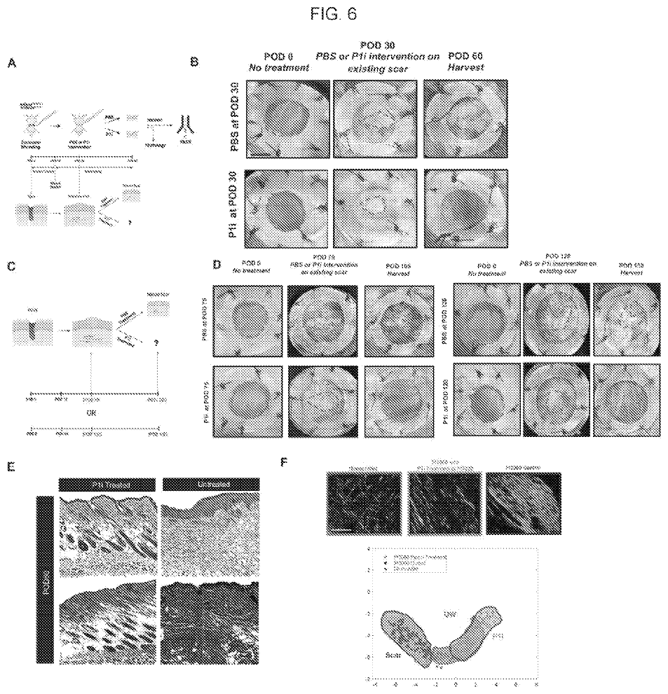

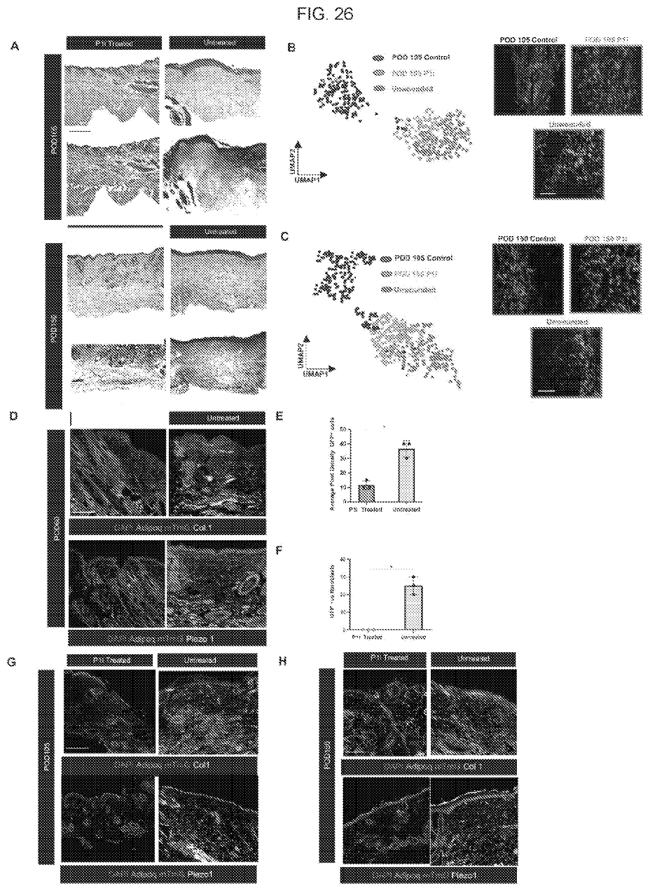

While wound re-epithelialization is complete relatively quickly (around 2 weeks), wounds continue to heal beneath the surface for months to years in a process known as scar remodeling. (Foster et al., 2021; Gurtner et al., 2008) Remodeling is the longest and least well understood phase of wound repair but is likely critical to ultimate outcomes. We postulated that ADFs could continue to play a role in scarring during the remodeling process; if this were the case, we wondered whether ADF activity could be targeted during wound remodeling, via P1i in existing, actively remodeling scars, to ultimately reduce scarring and drive regeneration (FIG. 6A,C). Thus, we administered P1i to existing scars at POD 30 (early remodeling period), POD 75 (mid-remodeling), and POD 120 (late remodeling), then harvested tissue one month later at POD 60, POD 105, and 120 respectively (FIG. 6A, C). Remarkably, treatment with P1i was sufficient to induce near-complete wound regeneration by POD 60, POD 105, and POD 120 with return of hair follicles (FIG. 6B, D), including full recovery of unwounded-like ECM architecture, compared to untreated wounds which remained scar-like on histology (FIG. 6E-F, 26A-C, 27-29). Of note, the hair follicles at POD 105 and 120 were less organized than those at POD 60 following P1i treatment suggesting the regeneration is less complete (FIG. 6E, 26A). ADFs were significantly reduced in late P1i-treated wounds (FIG. 26D-H), suggesting that P1i was still acting on ADFs even when given at this later timepoint. Treatment of POD 30 scars, with YAPi did not induce regeneration suggesting that adipocytes play a more significant role in the remodeling phase of wound healing than fibroblasts (30A-C). Overall, these findings suggested that ADFs continue to play a role during the remodeling phase, and that P1i could be effective to target ADFs and thereby promote regeneration even in existing scars.

### 7. Spatial Transcriptomics Defines Mechanically Sensitive Fibroblasts in Wound Healing

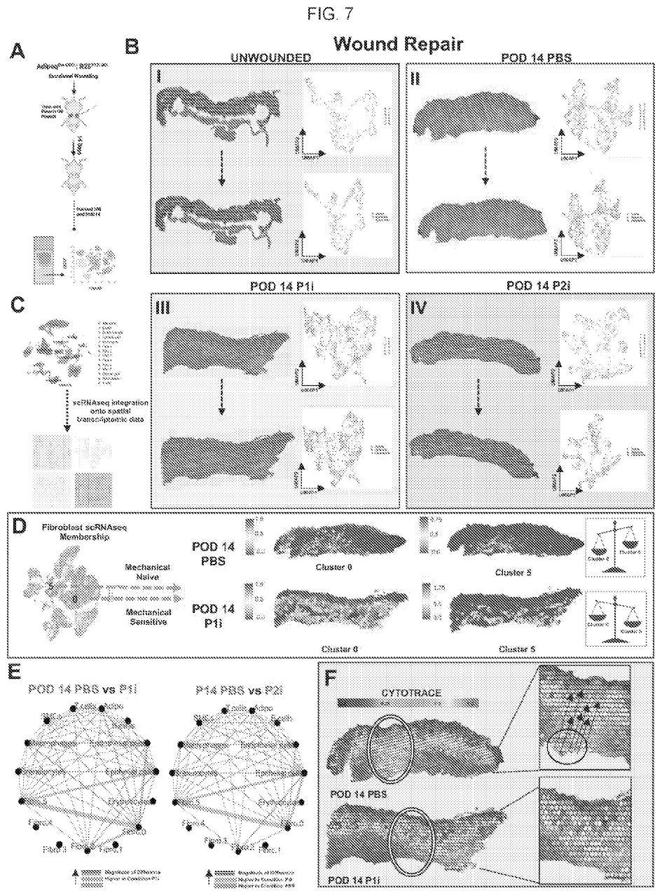

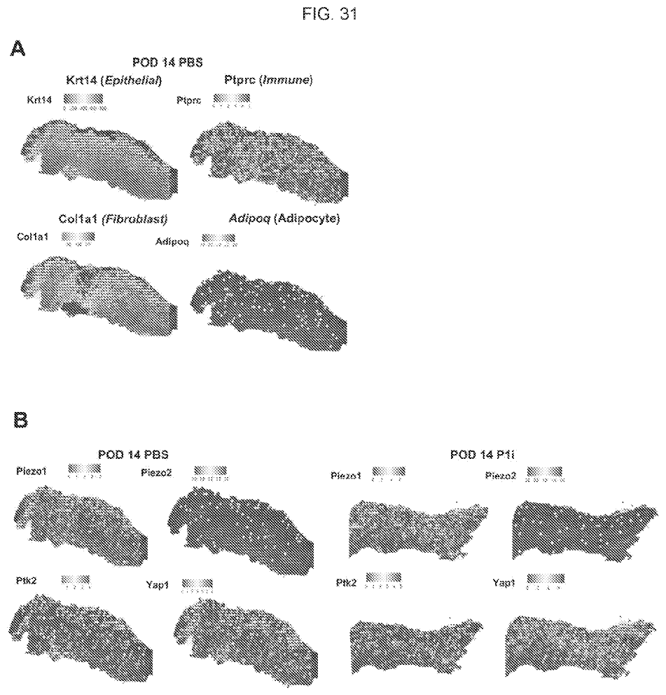

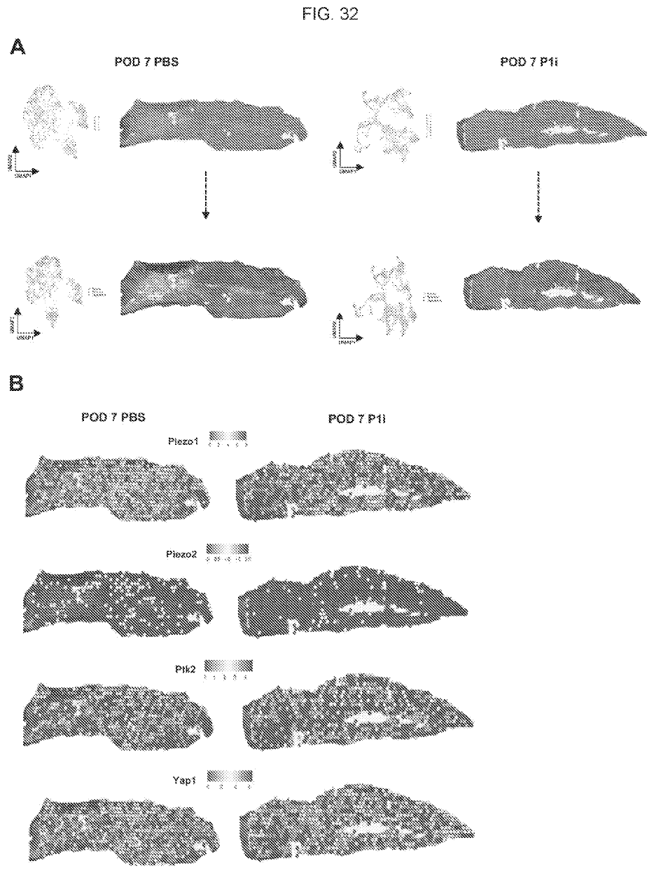

To further explore the significance of ADFs in wound repair, we applied the 10× Genomics Visium platform to analyze gene expression in the context of the spatial environment. We performed spatial transcriptomic analysis on histological sections from wounds at POD 14 (re-epithelized) with and without P1i/P2i, as well as unwounded skin (FIG. 7A). The epidermal, dermal, and hypodermal layers were clearly identified both histologically and according to their gene expression profiles (FIG. 7B). Furthermore, analysis of individual genes for well-known wound healing cell types, showed a clear distinction of keratinocytes in the epidermis (Krt14), fibroblasts and immune cells in the dermis (Col1a1 and Ptprc, respectively), and adipocytes in the hypodermis (Adipoq) as shown in wounds treated with PBS (FIG. 31A). POD 7 wounds were also easily histologically delineated according to the epidermis, dermis, and hypodermis (FIG. 32A).

To further elucidate the role of the mechanosensitive fibroblast population identified through scRNA-seq (FIG. 5), we evaluated the spatial expression of markers defining this population including Piezo1, Piezo2, Ptk2, and Yap1. In PBS treated wounds Piezo1, Piezo2, Ptk2, and Yap1 were found at the apical aspect of the wound, compared to P1i treated wounds where they appeared mostly at the basal region (FIG. 31B). A similar spatial pattern was observed at POD7 in PBS and P1i treated wounds (FIG. 32B). These data suggest that the genes defining the mechanosensitive fibroblast population (Piezo1, Piezo2, Ptk2, and Yap1) are spatially distinct in wounds treated with PBS versus those treated with P1i/P2i.

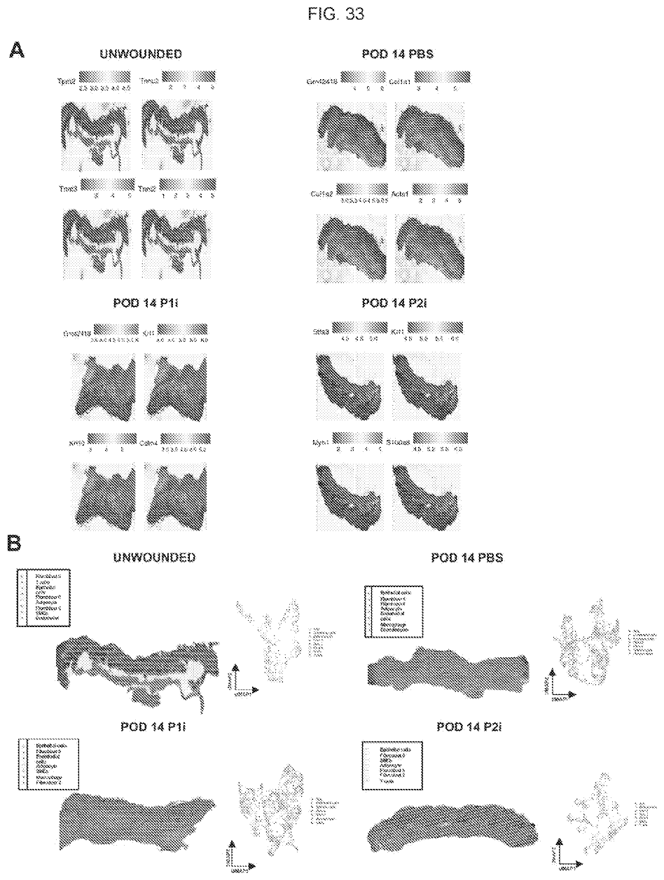

Analysis of spatially variable features across PBS and P1i/P2i treated wounds further illustrated the pro-fibrotic phenotype of PBS-treated wounds. The PBS treated wounds showed high expression of genes involved in scarring, including Co1a1a1, Col1a2, and Acta1 (FIG. 33A). In comparison, P1i/P2i treated wounds revealed high expression of markers associated with the epidermis including Krt1 and Krt14 (FIG. 33A).

To ascertain the spatial pattern of scRNA-seq defined populations at POD 14 (FIG. 5), we performed anchor-based integration to assess membership of each of the six scRNA defined fibroblast clusters (FIG. 7C). We found that the predicted spatial distributions for our scRNA-seq clusters were largely congruent with our observed differences in the distribution of mechanically sensitive and naïve populations (FIG. 7D, left). Fibroblast scRNA-seq cluster 0, our mechanically sensitive population, was highly enriched in PBS-treated POD 14 wounds compared to P1i or P2i treated wounds (FIG. 7D, right top). In comparison, fibroblast scRNA-seq cluster 5, our mechanically naive population was observed in P1i/P2i treated wounds (FIG. 7D, right bottom). Furthermore, adipocyte and epithelial cells were enriched in the spatial environment of P1i and P2i-treated wounds compared to PBS treated wounds at POD 14 (FIG. 33B).

To assess communication networks among cell types given the spatial environment in PBS and P1i/P2i treated wounds at POD 14, differential interaction maps were generated comparing the groups. P1i and P2i treated wounds revealed strong interactions between adipocytes and fibroblasts (FIG. 7E). In comparison, PBS-treated wounds showed strong interaction between fibroblasts subpopulations (FIG. 7E). Lastly, to assess the relative differentiation states of the cells in the wound given a spatial context, we applied Cyto TRACE to the four wound groups (FIG. 7F). We found greater transcriptional diversity from the apex compared to the basal dermis in a PBS treated scar, compared to wounds treated with P1i/P2i. 20 Collectively, these data suggest that mechanical inhibition through P1i or P2i can alter the spatial phenotype of adipocytes and fibroblasts during wound repair. Furthermore, the spatial pattern of ADFs and non-ADF subpopulations are distinct in wounds with regenerative and scarring phenotypes.

### 8. Spatial Phenotyping by CODEX Demonstrates Adipocyte-Fibroblast Cross-Talk in Wound Healing

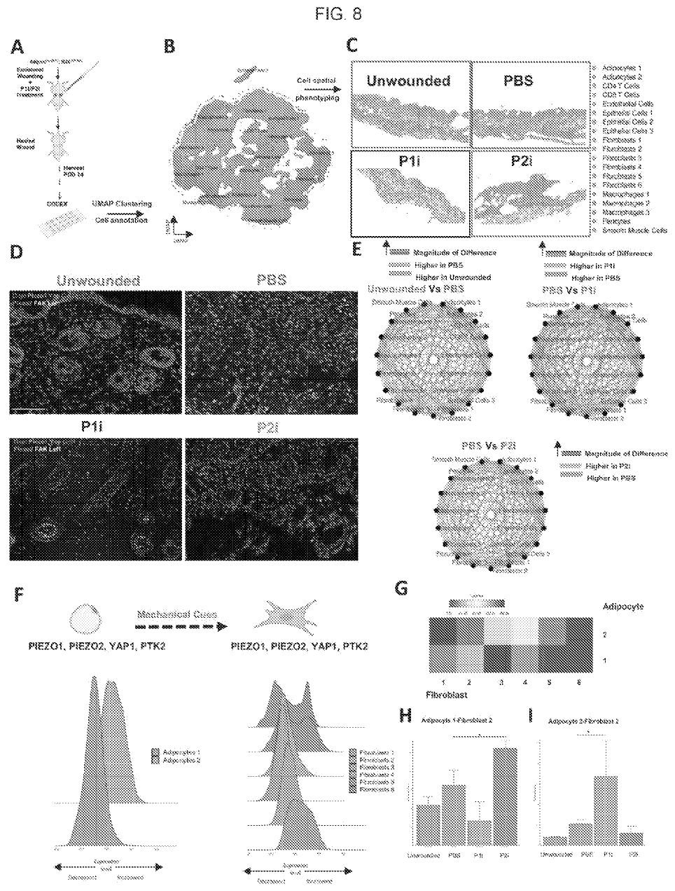

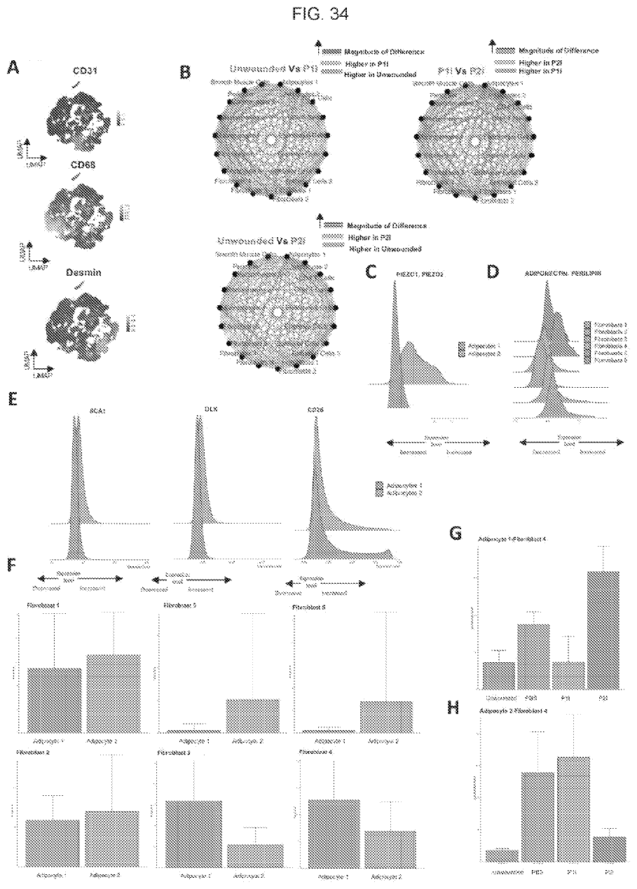

Building upon the spatial transcriptomic analyses described above, we further spatially phenotyped P1i-, P2i- and PBS-treated wounds at POD-14 (re-epithelized wounds) in a spatially-informed fashion at the protein level. We utilized CO-Detection by indEXing (CODEX), an assay in which a panel of 28+ individual protein markers are sequentially labeled with, and iteratively imaged via cyclic additions and washouts of dye-labeled oligonucleotide-conjugated antibodies (FIG. 8A; markers shown in Table 1, left column). Following denoising, image normalization, and automated cell segmentation, protein staining profiles were used to project a manifold of cell-representative clusters (FIG. 34A and FIG. 8B). Across all treatment groups, 19 cell clusters were identified on the basis of CODEX protein expression signatures, including subpopulations of six fibroblasts; three epithelial cells; five immune cells; two adipocytes, smooth muscle cells, endothelial cells, and pericytes (FIG. 8B-C). Broadly the expression of markers associated with mechanical signaling (PIEZO1, PIEZO2, YAP, and PTK2) was similar between P2i and P1i-treated wounds and unwounded skin, suggesting similar spatial protein expression (FIG. 8D). Notably, new hair follicle formation marked by Lef1 was highly expressed in P1i compared to PBS-treated wounds (FIG. 8D).

Differential interaction maps were then used to visualize the strength of spatial cell-cell interactions based on K-nearest-neighbor localization, which can be uniquely assessed using CODEX and other spatial phenotyping technologies. These spatial relationships were assessed in P1i-, P2i-, and PBS-treated wounds (FIG. 8E and FIG. 34B). PBS treated wounds showed strongly enriched interactions between adipocytes and fibroblasts compared to unwounded skin (FIG. 8E, top left). In comparison, P1i-treated wounds and to a lesser extent, P2i-treated wounds, exhibited reduced adipocyte-fibroblast interactions compared to PBS treated wounds (FIG. 8E, top right and bottom).

To further assess the role of mechanosensitive gene expression in the transition from adipocyte to fibroblast phenotypes in wound healing, a protein expression module consisting of PIEZO1, PIEZO2, YAP, and PTK2 was computed for all CODEX annotated cell types. Two adipocyte clusters were identified by CODEX. Adipocyte cluster 2 had greater mechanosensitivity, as defined by the co-expression of these markers, compared to adipocyte cluster 1 (FIG. 8F, left). When analyzing PIEZO1 and PIEZO2 co-expression alone, adipocyte cluster 2 also showed greater expression than adipocyte cluster 1 (FIG. 34C). The mechanosensitive signature of the six CODEX defined fibroblast subpopulations also varied, with clusters 1, 5, and 6 showing greater expression compared to clusters 2, 3, and 4 (FIG. 8F, right). Fibroblast clusters 1, 5, and 6 also displayed relatively higher co-expression of Perilipin and Adiponectin, suggesting these clusters to be ADFs (FIG. 34D). Furthermore, these putative ADFs were highly SCA1 positive, congruent with earlier findings (FIG. 34E, FIG. 1E).

Interestingly, the strength of cell-cell interactions between individual adipocyte and fibroblast subpopulations was also highly variable across the spatial microenvironment of wounds (FIG. 8G and FIG. 34F). The strongest adipocyte-fibroblast interactions were between fibroblast cluster 1 and adipocyte cluster 2, both of which are relatively mechanosensitive subpopulations (FIGS. 8F-G). P1i and P2i treatment also modulated the strength of individual adipocyte-fibroblast subpopulation interactions compared to control, PBS-treated wounds (FIG. 8H-I). P2i treatment enhanced interactions between mechanically naïve subpopulations including adipocyte cluster 1 and fibroblast clusters 2 and 4, relative to PBS-treated wounds (FIG. 8H and FIG. 34G). In comparison, P1i-treated wounds shifted the interactions of mechanically sensitive adipocytes to co-localize more with mechanically naïve fibroblasts instead of mechanically sensitive fibroblasts, specifically enriching the interactions of adipocyte cluster 2 with fibroblast clusters 2 and 4 (FIG. 8I and FIG. 34H).

### 9. Integration of RNA and Protein Spatial Phenotyping Modalities During Wound Healing

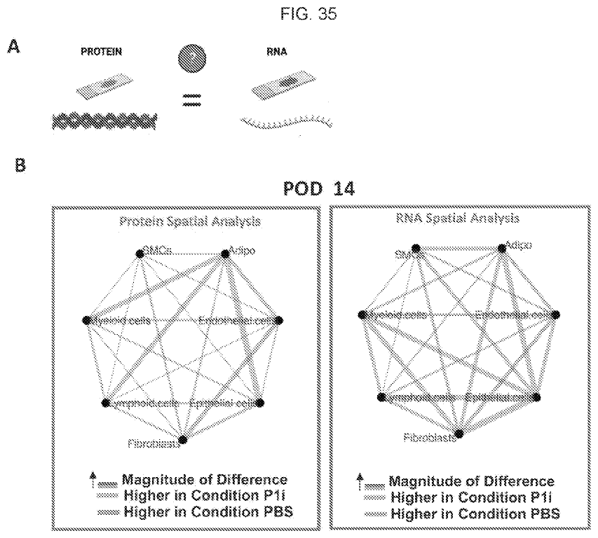

To understand if protein- and RNA-based spatial phenotyping revealed similar trends in cell spatial organization during wound repair, we compared cell-cell interactions using both techniques at POD 14 (FIG. 35A). Subclusters of cell types (e.g. Fibroblast cluster 1 and Fibroblast cluster 2) were combined into broad overlapping cell phenotypes to ensure that parallel cell populations and cell-cell networks could be compared using Visium and CODEX analysis. Interestingly, the cell-cell interactions in PBS versus P1i treated wounds at POD 14 were largely similar when evaluating between Visium and CODEX modalities (FIG. 35B). These data highlight the congruency in spatial analysis at an RNA and protein level during wound healing.

Collectively, these data suggest that P1i and P2i treatment differentially modify cell spatial organization in their mechanisms of supporting wound regeneration—in particular, by modulating interactions between mechanically defined adipocyte and fibroblast subpopulations.

### 10. Spatial Transcriptomics Supports Adipocyte to Fibroblast Transition in Rescuing Existing Scars

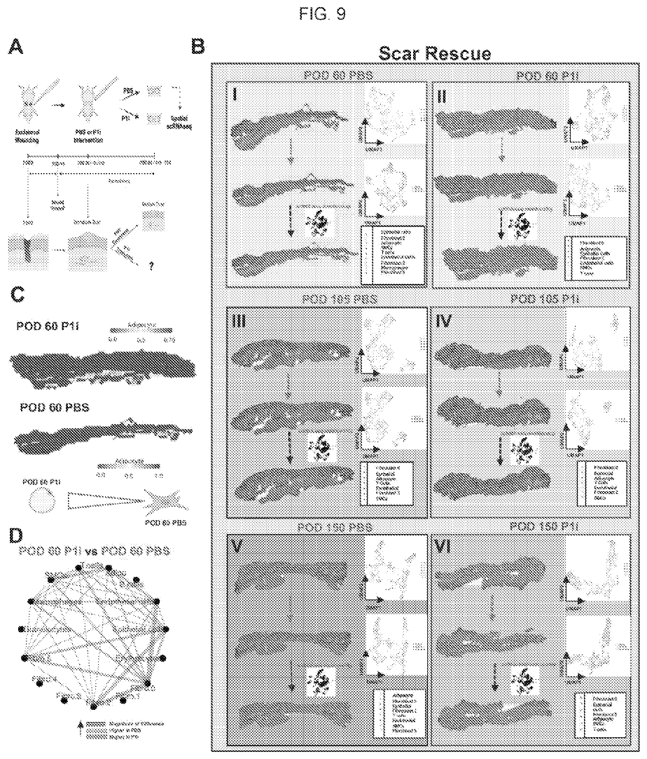

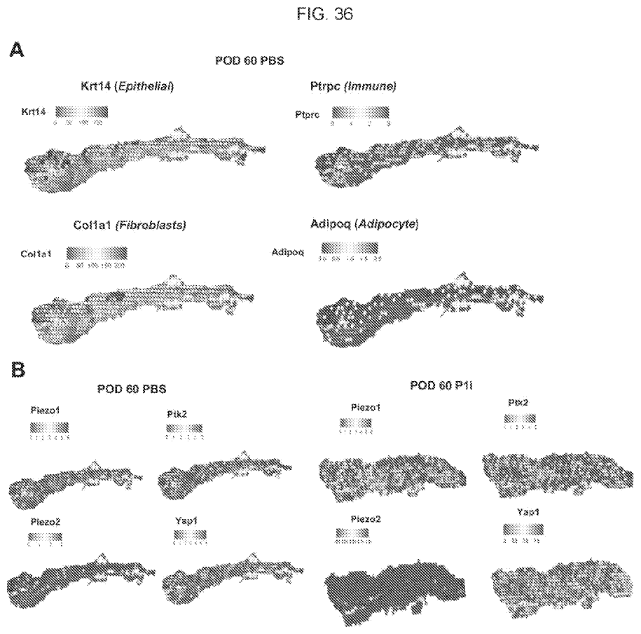

To further analyze the spatial gene expression following scar rescue, the Visium platform was applied to scars treated with P1i or PBS at POD 30, 75, and 120 (FIG. 9A). Similar to analysis of acute wound healing, the epidermal, dermal, and hypodermal layers were easily elucidated from the spatial transcriptomic analysis in scars at POD 60 (FIG. 9B). Furthermore, the expected cell types including fibroblasts, immune, epithelial, and adipocytes were identified by spatial expression of representative genes (FIG. 36A).

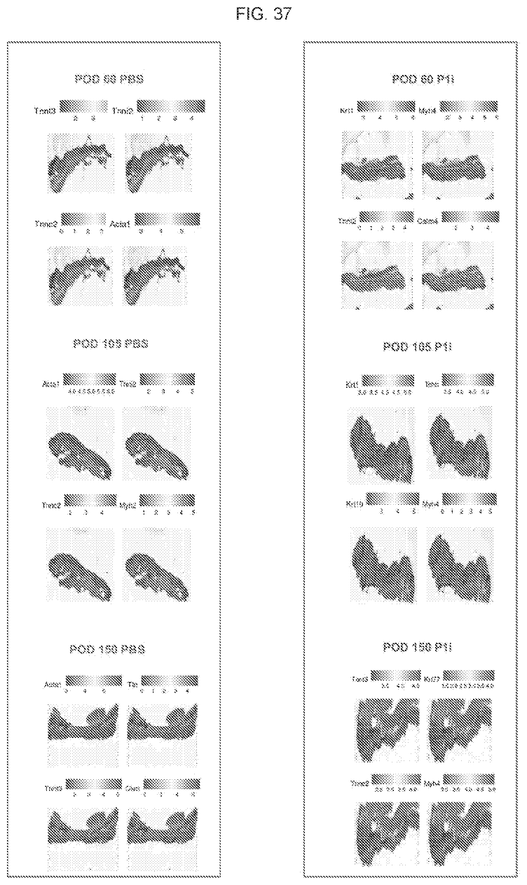

Interestingly, the most differentially expressed genes during wound rescue following PBS or P1i treatment were similar to those defined during wound repair. P1i treated wounds showed high expression of genes associated with the epidermis, whereas PBS treated wounds revealed elevated expression of genes known to be associated with scarring (FIG. 37). To elucidate the spatial patterning of genes associated with the mechanically sensitive fibroblast populations, the spatial patterning of Piezo1, Piezo2, Yap1, and Ptk2 was evaluated across all timepoints (FIG. 36B). In P1i treated wounds the expression of Piezo1, Piezo2, Yap1, and Ptk2 were observed mostly at the basal aspects of the wounds compared to PBS treated wounds where they were found to be in the apical part of the wound.

To further clarify the role of ADFs in the spatial environment of established scars, the scRNA-seq defined cell populations were again anchor transferred onto the spatial transcriptomic analysis of histological sections (FIG. 9B). Over early (POD 60), mid remodeling (POD 105), and late remodeling (POD 150) the spatial proportions of adipocytes and fibroblasts varied with PBS and P1i treatment (FIG. 9B). The spatially identified adipocytes were greater in number in the P1i treated scars compared to PBS treated scars at POD 60, 105, and 150 (FIG. 9C). These data suggest that the adipocyte-fibroblast transition is not only transcriptionally evident at the single cell level but also distinct in the context of the spatial organization of the tissue.

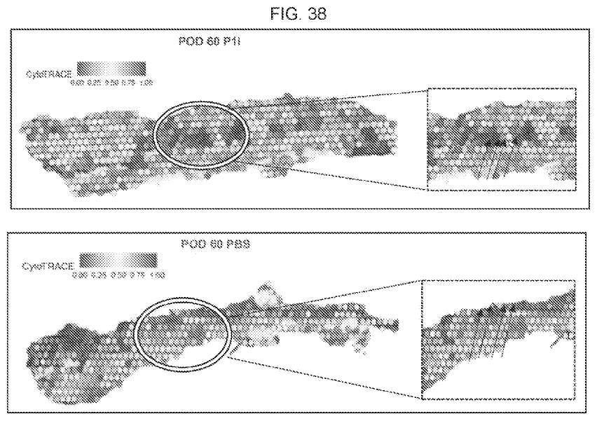

To assess spatial communication of cell types in the scars, differential interaction maps were compared between the seven groups (FIG. 9D). Interestingly, P1i-treated wounds showed a larger number of interactions between adipocytes and epithelial cells, suggestive of regenerative communications (FIG. 9D). In comparison, PBS-treated wounds revealed a relatively higher number of adipocyte-fibroblasts communications, suggestive of possible fibrotic communications (FIG. 9D). Lastly, CytoTRACE further revealed, as observed during wound healing (FIG. 7), greater transcriptional diversity from the apical dermis compared to the basal dermis in a PBS treated scar compared to a P1i-treated scar (FIG. 38).

Collectively, these data suggest that the adipocyte-fibroblast crosstalk, may play an important role in the spatial organization of pre-existing scars. Furthermore, mechanotransduction modulation may alter the spatial organization of the adipocyte to fibroblast transition in a way that facilitates the regeneration of established scars.

### 11. Spatial Phenotyping by CODEX Demonstrates Adipocyte-Fibroblast Cross-Talk in Rescuing Existing Scars

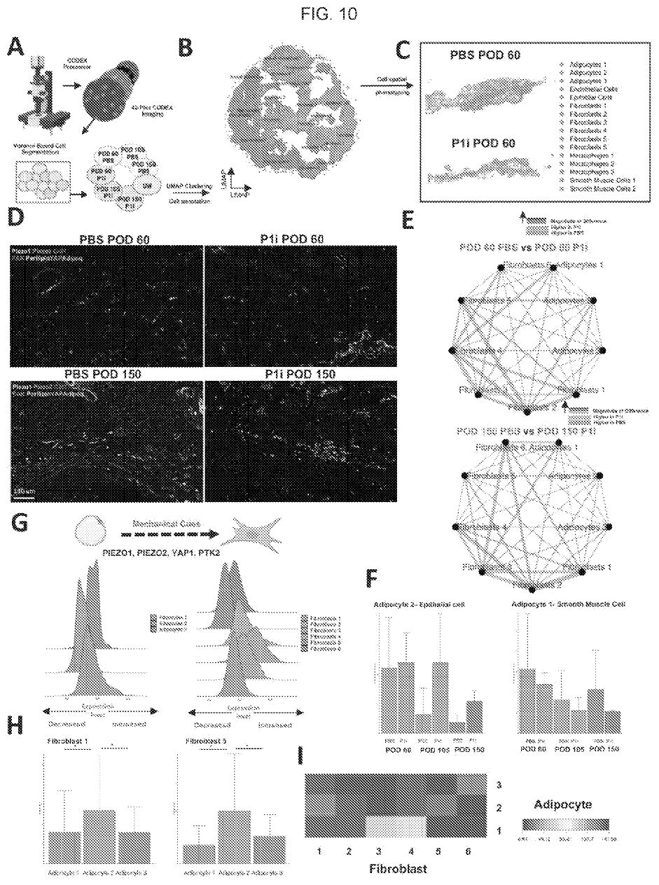

To phenotype the spatial environment in the context of rescuing existing scars, CODEX was further employed to spatially define protein expression, cell populations, and associated cell-cell interactions (FIG. 10A; markers shown in Table 1 middle column, 39A). We examined the spatial microenvironment of scars treated with P1i or PBS at POD 30 (early remodeling period) and harvested at POD 60, treated with P1i or PBS at POD 75 (mid-remodeling) and harvested at POD 105, and treated with P1i or PBS at POD 120 and harvested at POD 150 (late remodeling) (FIG. 6). Across all treatment groups, a manifold of 16 clusters was identified based on protein expression, including multiple subpopulations of adipocytes, fibroblasts, epithelial cells, smooth muscle cells, endothelial cells, and macrophages (FIG. 10B-C). PBS treated wounds showed higher expression of mechanical markers at POD 60, 105, and 150 compared to those treated with P1i (FIG. 10D). Differential interaction maps were then used to visualize spatially defined cell-cell interactions in P1i- and PBS-treated scars (FIG. 10E). Most notably, the interactions of adipocyte and fibroblast subpopulations varied between PBS and P1i treated scars at POD 30 (harvested at POD 60), 75 (harvested at POD 105), and 120 (harvested at POD 150) (FIG. 10E, 39B-D). P1i treated scars showed enriched interactions mediated by adipocyte cluster 2 compared to PBS treated scars (FIG. 10E). On the other hand, PBS treated wounds showed enriched interactions mediated by adipocyte clusters 1 and 3 (FIG. 10E). Following P1i treatment, adipocyte cluster 2 interacted more strongly with epithelial cells, implicating regenerative communication, at POD 60, 105, and 150 (FIG. 10F, left). In comparison, following PBS treatment, adipocyte cluster 1 interacted more strongly with smooth muscle cells, suggestive of inflammatory and scar-associated communication (FIG. 10F, right).

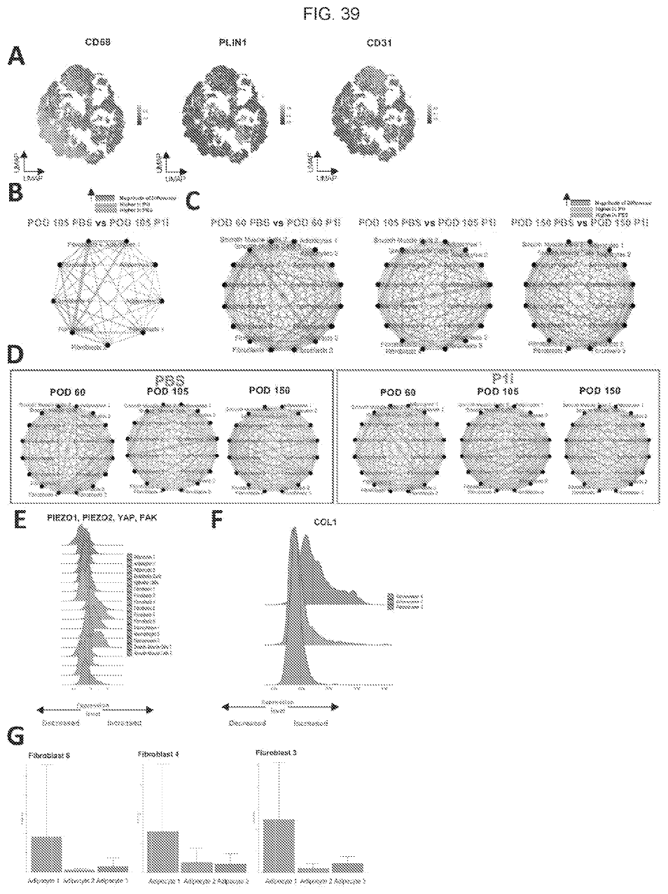

To further assess the role of mechanosensitive protein expression in adipocyte-fibroblast transition during scar rescue, a protein module of PIEZO1, PIEZO2, YAP1, and PTK2 was again analyzed in all CODEX annotated cell types (FIG. 39E, left). Adipocyte 1 and 3 had the greatest mechanical sensitivity, as defined by the co-expression of these markers compared to adipocyte cluster 2 (FIG. 10G, left). CODEX defined adipocyte cluster 3 also demonstrated the greatest collagen type I expression, indicative of a fibrotic phenotype (FIG. 39F). CODEX further revealed variability in the mechanical signature of the fibroblast clusters during scar rescue. Fibroblast clusters 2, 3, 4, and 6 showed greater mechanical sensitivity compared to clusters 1 and 5, indicative of a more fibrotic phenotype (FIG. 10G, right). Most interestingly, across the timepoints, the pro-regenerative mechanically naïve populations (Fibroblast 1 or 5 and Adipocyte 2) and pro-fibrotic mechanically sensitive populations (Fibroblast 2, 3, 4, 6 and Adipocyte 1 or 3) showed high levels of intra-group interactions (FIG. 10H-I, 39G). Collectively, CODEX spatial protein analysis suggested that signaling between adipocytes and fibroblasts markedly occurs during the rescue of existing scars. The extensive cross-talk observed between profibrotic, mechanically sensitive adipocytes and fibroblasts suggests that targeted mechanical modulation, which can be achieved by P1i, underlies wound regeneration during scar rescue.

### 12. Piezo Inhibition Reduces Scarring in a Human Foreskin Xenograft Wound Model

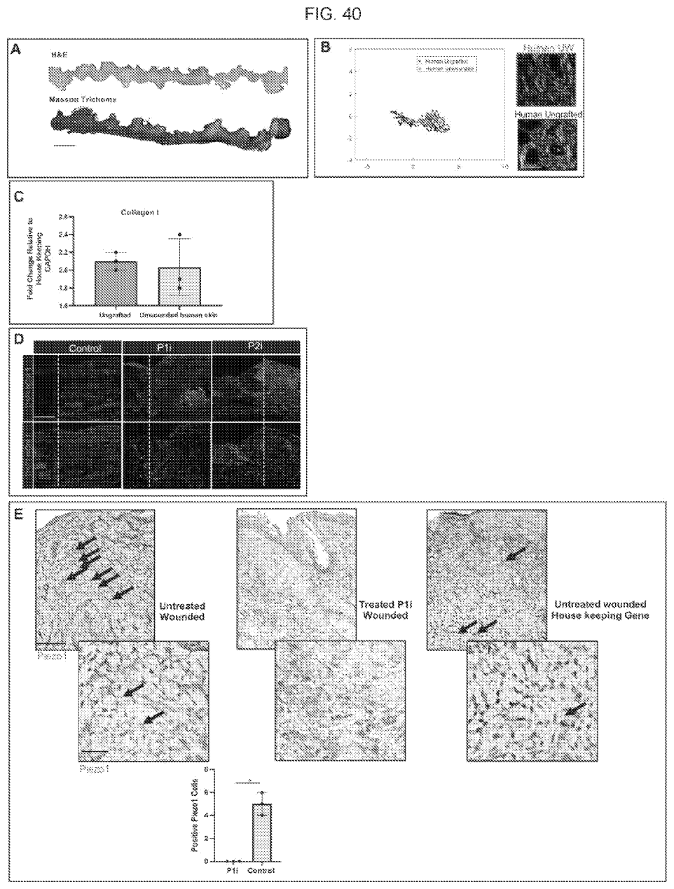

Finally, while our results in mice were consistent with Piezo1 or Piezo2 blockade preventing adipocyte-to-fibroblast transition and reducing scarring, it is ultimately critical to determine whether similar anti-scarring effects are seen in human wounds. We developed a xenograft model to study healing by human cells, wherein human foreskin samples were engrafted onto CD-1 nude recipient mice then underwent full-thickness wounding (FIG. 11A). Unwounded grafted skin had similar histologic appearance, ECM ultrastructure, and Col1 expression compared to non-grafted skin (FIG. 40A-C), supporting that engraftment itself did not cause a fibrotic reaction within foreskin tissue. Following wounding, gross and histologic examination confirmed that distinct fibrotic scars formed within healed wounded xenografts (FIGS. 11B, D and 40D). IF for human and mouse-specific Col1 confirmed that xenografted human versus adjacent mouse skin were distinguishable on the basis of collagen and that the xenografts healed with human collagen (FIGS. 11G, top left panel, and 40D), consistent with wound repair by human cells within xenografts (as opposed to wound repair by mouse cells from surrounding tissue). We next treated xenograft wounds with P1i or P2i. As in mice, Piezo inhibition grossly and histologically reduced scarring (FIG. 11B-E). ECM ultrastructure of P1i wounds was indistinguishable from that of unwounded xenograft skin (FIG. 11F) and Piezo inhibitor-treated scars had reduced collagen and increased adiponectin content (FIG. 11G-H). Piezo-inhibited wounds also had increased staining for CD31 (suggesting increased vascularization) and CK19 (consistent with regenerated secondary elements, e.g., Fordyce/oil glands; FIG. 11I-J). RNAscope in situ hybridization confirmed fewer Piezo1⁺ cells when Piezo1 was inhibited (FIG. 40E). Collectively, these results suggest that Piezo1 inhibition in human skin wounds yielded wound regeneration with minimized scarring.

13. Mechanically Naïve Human Fibroblasts are Identified in P1i Treated Xenografts Using scRNA-Seq

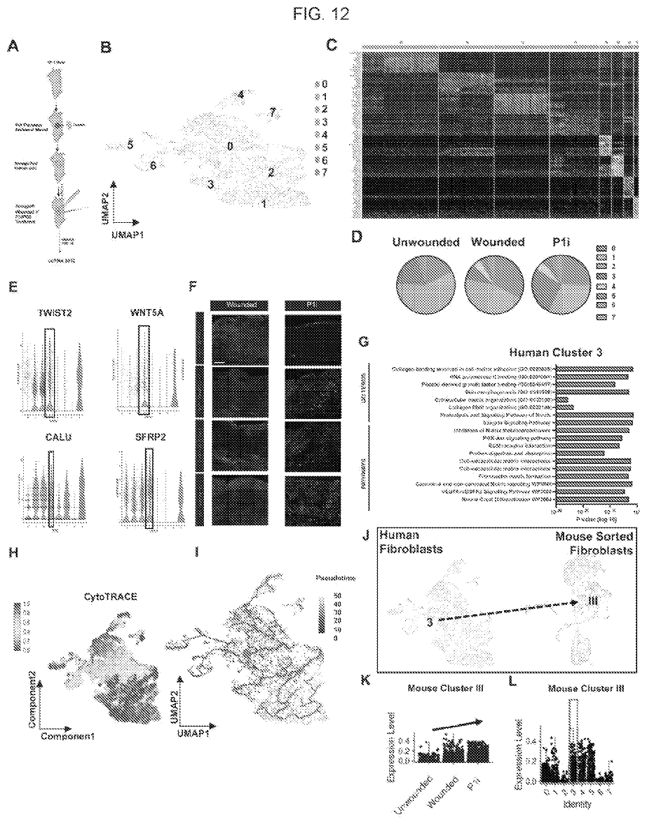

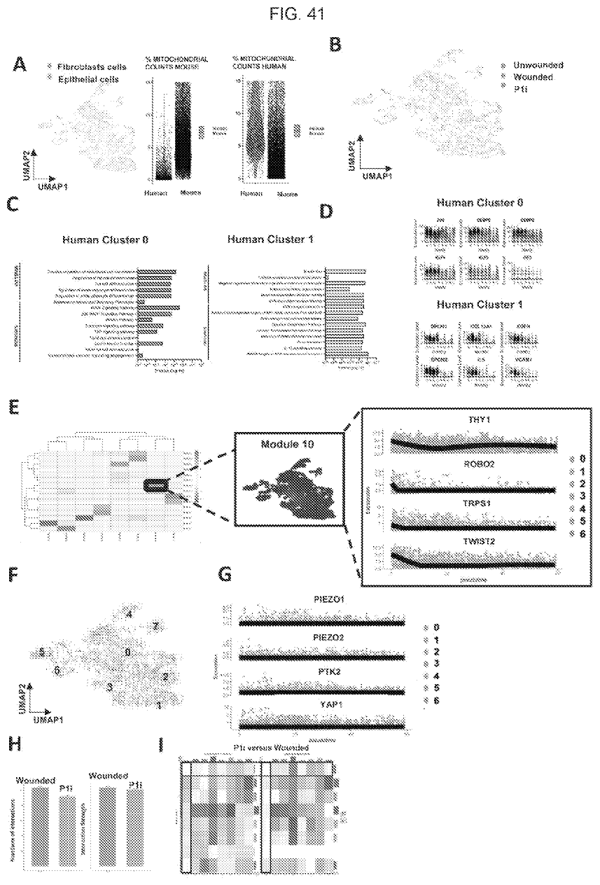

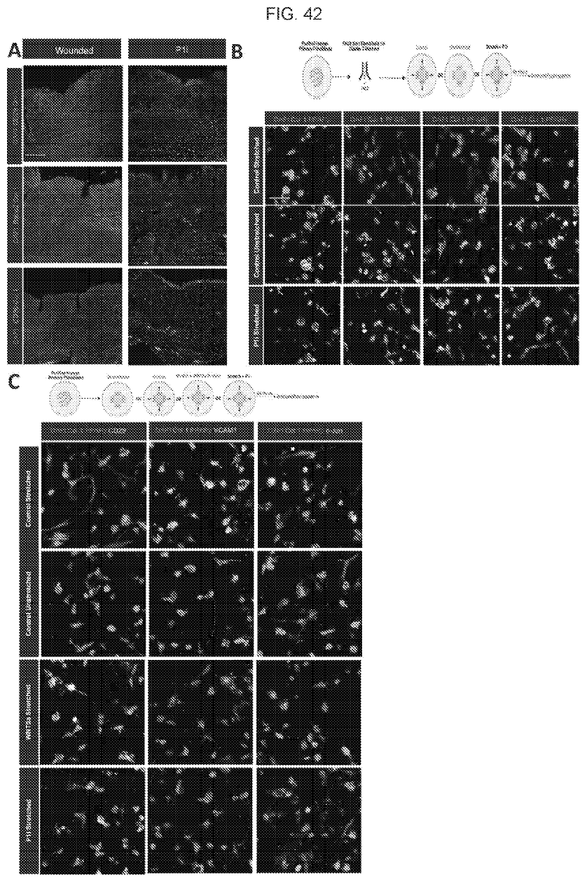

To assess if P1i acts on similar fibroblast clusters to cause regeneration, scRNA-seq analysis was performed on xenografts harvested at POD 14 post injury using a hybrid human-mouse xeno-transcriptome (FIG. 12A). Mouse cells were removed informatically and subsequent analysis focused exclusively on human cells (FIG. 41A). Partitional analysis identified 8 transcriptionally distinct clusters (FIG. 12B-C), corresponding to fibroblast and keratinocyte populations (FIG. 41A). Each cluster was present in all three treatment conditions but Cluster 3 was elevated in P1i treated wounds (FIG. 12D, FIG. 41B). Interestingly, cluster 3 was characterized by elevated expression of pro-regenerative genes such as TWIST2, WNT5A, CALU, and SFRP2 (FIG. 12E F) which were found to be highly expressed in P1i-treated wounds (FIG. 12F and FIG. 42A). This cluster was also enriched for pathways such as “skin morphogenesis”, as well as “neural crest differentiation” (FIG. 12G). By contrast, Cluster 0 was enriched for pathways related to adipocyte differentiation (FIG. 41C) and elevated expression of genes related to adipocyte-fibroblast transition (FIG. 41C) including expression of JUN a transcription factor associated with fibrosis (FIG. 41D).

CytoTRACE analysis identified significant differences in the differentiation state of cluster 3 cells compared to other fibroblast clusters (FIG. 12H), allowing us to construct pseudotime trajectories from this cluster (FIG. 12I). This demonstrated significant changes in the expression of key genes such as THY1, ROBO2, TRPS1, and TWIST2, throughout the putative transition of cells from cluster 3 to other subgroups (FIG. 41E). Pseudotime analysis suggested a trajectory from cluster 3, to cluster 0 (associated with JUN expression), and then to cluster 1 (associated with VCAM1 expression) (FIG. 12I and FIG. 41C-D). These candidate trajectories were also supported by RNA velocity analysis (FIG. 41F). Furthermore, mechanosensitive genes (PIEZO1, PIEZO2, YAP1, and PTK2) were highly expressed over these pseudotime trajectories (FIG. 41G). CellChat also demonstrated a downregulation of cluster 0 activity with other clusters following P1i compared to control wounds (FIG. 41H-I).

Interestingly, to evaluate these clusters in the context of our previously-defined fibroblast populations, we performed cross-species mapping using a label transfer-based approach (FIG. 12J-L), which suggested that cluster 3 cells were of highest similarity to our mechanically-naive mouse fibroblast III cells. These data suggest that mechanically naïve fibroblast populations may exist during human wound healing.

### 14. Mechanical Cues May Convert Human Regenerative ADFs Towards Fibrotic ADFs

To further confirm that human cluster 3 represented a pro-regenerative ADF population we utilized our in vitro collagen gel system. We first, FACS sorted human fibroblasts based on expression of cluster 3 markers including ROBO2, CD29, and SFRP2 and seeded them into 3D collagen gels (FIG. 42B). Upon stretching for 48 hours, these fibroblasts demonstrated high expression of typical fibroblast markers including Collagen Type I and low expression of typical adipocyte markers including PPARg using IF analysis (FIG. 42B). In contrast, stretching with P1 inhibition kept the fibroblasts in a more adipocyte-like phenotype, with high expression of PPARg and low expression of Collagen Type I, similar to fibroblasts in an unstretched state (FIG. 42B). These data suggest that mechanotransduction may induce the phenotype of the regenerative human cluster 3 towards a more fibrotic phenotype.

The scRNA analysis also demonstrated high expression of Wnt5A and Calu in human fibroblast cluster 3, both of which are known to play a role in the Wnt signaling pathway. To determine if P1i may activate canonical Wnt pathways to maintain cluster 3 in an adipocyte-like phenotype, we applied Wnt5a recombinant protein to the in vitro collagen gel system (FIG. 42C). As expected upon stretching of human fibroblasts displayed low expression of regenerative cluster 3 marker, CD29 but high expression of Cluster 0 (CJUN) and 1 (VCAM1) markers (FIG. 42C). In contrast, in the presence of either P1i or Wnt5a recombinant protein human fibroblasts demonstrate a more adipocyte like phenotype with high expression of PPARg and low expression of Collagen Type 1 (FIG. 42C). In summary, P1i treatment may revert fibroblasts towards the mechanically naïve fibroblast pro-regenerative fibroblast population through the activation of canonical Wnt pathways and thereby preventing the transition to pro-scarring phenotypes defined by CJUN and VCAM1 expression.

### 15. Spatial Proteomic Phenotyping by CODEX Reveals Adipocyte Plasticity in Wound Repair

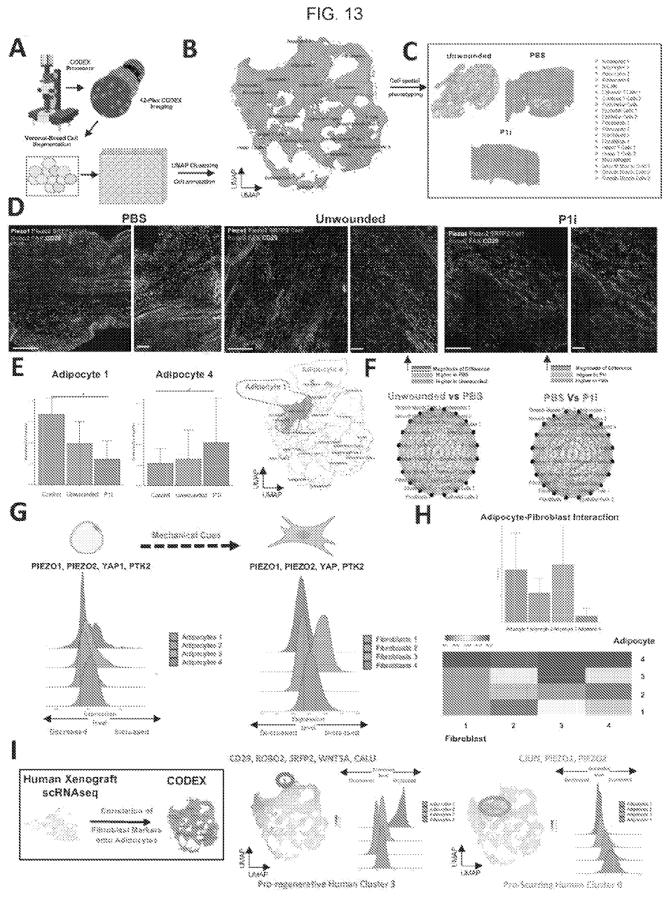

While scRNA-seq of human xenografts investigates human fibroblast subpopulations at a transcriptomic level, we further utilized CODEX to analyze spatially defined protein expression and cellular organization (FIG. 13A; markers shown in Table 1 right column). Specifically, we compared the spatial microenvironment of PBS, P1i, and unwounded skin at POD 14. Across all treatment groups, a manifold of 20 clusters was identified based on protein expression, including multiple subpopulations of adipocytes, fibroblasts, epithelial cells, smooth muscle cells, helper T cells, cytotoxic T cells, endothelial cells, B cells, and macrophages (FIG. 13B, 43A). Broadly, the spatial pattern of the protein markers was similar between P1i treated wounds and unwounded skin (FIG. 13B-C). Interestingly, CODEX analysis identified four adipocyte subpopulations based on protein expression (FIG. 13B). We thus compared the representation of adipocyte subtypes within P1i-versus PBS-treated wounds (FIG. 13E, 43B). The relative proportions of CODEX defined adipocyte clusters significantly differed based on wound treatment, with fewer of the adipocyte 1 subtype and more of the adipocyte 4 subtype in P1i-treated wounds compared to PBS treated wounds (*p<0.05; FIG. 13E, 43B).

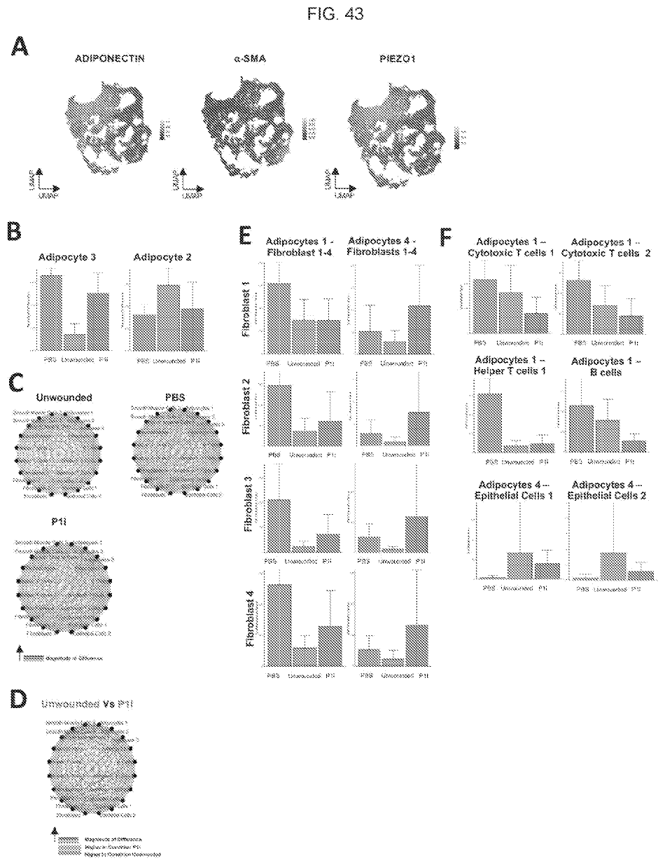

Differential interaction maps were then used to visualize spatially defined cell-cell interactions in P1i- and PBS-treated wounds (FIG. 13F), particularly for the adipocyte 1 and 4 subpopulations that were differentially represented across treatments. PBS-treated wounds showed broadly enriched interactions between fibroblasts and adipocytes compared to P1i-treated wounds (FIG. 13F and FIG. 43C-E). Interactions by individual adipocyte clusters also varied among the three treatment groups. PBS-treated wounds showed highly enriched interactions between adipocytes 1 and various immune cells (e.g., cytotoxic T cells, helper T cells, B cells), suggestive of inflammatory and scar-associated communication (FIG. 43F). In contrast, P1i-treated wounds showed strong crosstalk between adipocytes 4 and epithelial cells, suggestive of regenerative interactions (FIG. 43F).

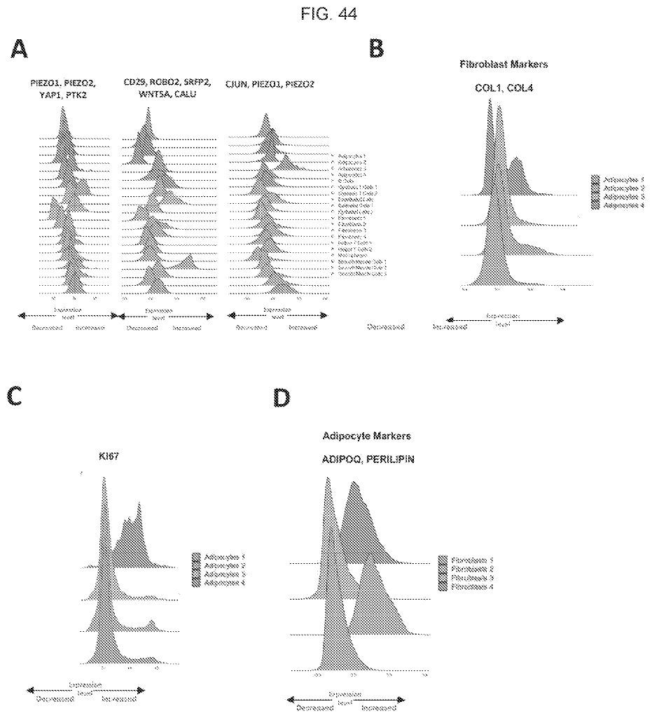

To further assess the role of mechanosensitive expression in the transition from adipocyte to fibroblast phenotypes, a protein module of PIEZO1, PIEZO2, YAP, and PTK2 was analyzed in all CODEX annotated cell types (FIG. 44A). Adipocyte and fibroblast subpopulations had varying levels of mechanical sensitivity, as defined by the co-expression of these markers (FIG. 13G). Interestingly, adipocyte clusters 1 and 3 exhibited strong protein expression of PIEZO1, PIEZO2, YAP, and PTK2 suggesting that these clusters have a more mechanically activated phenotype compared to adipocyte clusters 2 and 4 (FIG. 13G). On the other hand, fibroblast clusters 1 and 3 had greater mechanosensitive expression compared to fibroblast clusters 2 and 4 (FIG. 13G).

We also analyzed a canonical fibroblast-like (COL1, COL4) protein expression module in each of the adipocyte subpopulations to infer relative transitional status. Adipocyte cluster 4 had the lowest expression of fibroblast-like markers (COL1, COL4) compared to adipocyte clusters 1, 2, and 3, indicating that adipocyte cluster 4 may represent a “mechanically naïve”, less fibroblast like state compared to adipocytes 1, 2, and 3 (FIG. 44B). Interestingly, adipocyte cluster 4 demonstrated a bimodal expression profile for both mechanical sensitivity (PIEZO1, PIEZO2, YAP1, and PTK2; FIG. 13G) and fibroblast-like proteins (COL1, COL4; FIG. 44B), in addition to high Ki67 expression (FIG. 44C), suggesting that a subset of this cluster may be transitioning towards a more fibroblast-like phenotype and further underscoring the observed plasticity of adipocytes. Adipocyte cluster 1 and 3 also interacted more extensively with fibroblasts in the spatial microenvironment compared to adipocytes 2 and 4 (*p<0.05) (FIG. 13H, top panel). Taken together, these data suggest that mechanically sensitive adipocytes have higher expression of fibroblast-like markers and enriched spatial interactions with fibroblasts.

We similarly analyzed a canonical adipocyte-like (ADIPOQ, PERILIPIN) protein expression module in each of the fibroblast subpopulations to analyze relative transitional status. Fibroblast clusters 1 and 3 had lower expression of adipocyte-like markers compared to fibroblast clusters 2 and 4 (FIG. 44D), as well as relatively higher mechanosensitive protein expression (PIEZO1, PIEZO2, YAP1, and PTK2; FIG. 13G). Fibroblasts cluster 1, the subtype with the greatest mechanosensitive expression, had the greatest degree of interaction with adipocyte clusters 1, 2, and 3 but not adipocyte cluster 4 (FIG. 13H, bottom panel). Thus, mechanically sensitive fibroblasts demonstrated lower expression of adipocyte-like markers, but a higher degree of spatial interaction with adipocytes. Overall, our analyses of CODEX-defined fibroblasts and adipocytes established a strong association between mechanically activated protein expression, enhanced adipocyte-fibroblast spatial interactions, and further transition from adipocytes to fibroblasts.

Lastly, we mapped the pro-scarring (CJUN/PIEZO1/PIEZO2+) and pro-regenerative (ROBO2/SFRP2/WNT5A/CALU+) ADF clusters from our human xenograft scRNA-seq analysis to the CODEX dataset (FIGS. 13I left and 44A middle and right). Specifically, CODEX-defined adipocytes (PERILIPIN+ADIPOQ+) were interrogated using expression modules of the aforementioned markers for pro-regenerative scRNA-seq cluster 3 and pro-scarring scRNA-seq cluster 0 (FIGS. 13I left and 44A middle and right). Interestingly, CODEX adipocyte cluster 4, which demonstrated strong epithelial cell spatial interactions, mapped closely to the putatively pro-regenerative scRNA-seq fibroblast cluster 3 (FIG. 13I middle). CODEX adipocyte cluster 1, which demonstrated strong immune-adjacent and inflammatory interactions, mapped closely to the putatively pro-scarring scRNA-seq fibroblast cluster 0 (FIG. 13I right). This further suggests that human ADF scRNA-seq clusters 3 and 0, which display regenerative and fibrotic expression, respectively, may be derived from adipocytes with similar protein expression profiles. In summary, CODEX spatial protein analysis suggested that adipocytes display heterogeneity in their expression of mechanosensitive proteins, which produces distinct effects on their spatial interaction niche and their expression of markers along the adipocyte-to-fibroblast transition. Furthermore, fibrotic and regenerative markers identified by scRNA-seq strongly correlate with distinct protein-defined adipocyte subtypes. Collectively, these results suggest that mechanical cues are critical to facilitating the adipocyte to fibroblast transition in human wound repair and fibrosis.

### E. DISCUSSION

Overall, we have shown via adipocyte engraftment and genetic lineage-tracing studies that mature adipocytes lose adipogenic markers and transition to fibroblasts in wounds. Using both in vitro and in vivo models to modulate adipocyte mechanical environment, as well as single-cell transcriptomic analysis in Rainbow mice, we demonstrated that transition of both mouse and human adipocytes into adipocyte-derived fibroblasts is a mechanically-driven process, differentiating via a “mechanically naïve” fibroblast intermediate and involving signaling by mechanosensitive ion channel (Piezo) genes (FIG. 45). These results elucidate a molecular signature and role of adipocytes and adipocyte-fibroblast dynamics in contributing to dermal fibrosis during wound healing, and highlight a novel mechanism through which wound mechanics drive fibrosis and govern adipocyte fate. Finally, we found that blocking adipocyte mechanotransduction via Piezo1 inhibition prevents adipocyte-to-fibroblast transition and can both prevent and rescue scarring to yield wound regeneration, a result which indicates that adipocyte-fibroblast differentiation is a meaningful contributor to scarring and can be targeted to prevent fibrosis.

While adipocytes were previously proposed to give rise to fibroblasts in the context of scarring, the precise role and drivers of this process were incompletely understood. Our findings place adipocyte-derived fibroblasts into the context of known dermal fibroblast heterogeneity, which may have implications for their function in wound healing. Prior reports have demonstrated that hypodermal/lipofibroblasts are capable of differentiating into adipocytes and vice versa; (Driskell et al., 2013; Plikus et al., 2017; Shook et al., 2020) our findings provide evidence for lineage plasticity in the latter direction and specifically suggest that adipocytes transition to lipofibroblasts in wounds. Importantly, previous studies suggest that lower and hypo-(vs. upper) dermal fibroblasts are less capable of contributing to HF regeneration and instead more prone to fibrotic ECM deposition. (Driskell et al., 2013) Collectively, these attributes are consistent with our findings suggesting that adipocyte-derived wound fibroblasts may have a pro-scarring phenotype and thus be candidate targets for reducing scarring.

Such functional heterogeneity among wound fibroblasts could also help to explain why, despite the fact that adipocyte-derived fibroblasts only comprise ˜10% of all scar fibroblasts, targeting adipocyte-to-fibroblast transition was able to significantly reduce scarring, as adipocyte-derived fibroblasts may be particularly pro-fibrotic. While adipocytes have been relatively less studied as cellular culprits of fibrosis (compared to fibroblasts), clinical findings across multiple disease settings have long suggested that fat may be linked to fibrosis. For instance, loss of fat is well known to be a feature of the systemic fibrotic disease systemic sclerosis (SSc; scleroderma) and fat grafting has been proposed to have pro-regenerative effects in SSc; (Strong et al., 2019) in the bowel, “creeping fat” is a finding intimately associated with intestinal fibrosis in Crohn's disease. (Dickson, 2020) Our findings in this study support that there may be a balance between adipogenic versus fibrotic cell fate of wound-associated adipocytes, which is altered by injury-associated molecular cues such as mechanical signaling. Importantly, the results of our KO and small molecule inhibitor studies suggest that by intervening on those molecular signaling pathways, we can modulate adipocytes toward a more favorable (adipogenic) cell fate and away from damaging fibrotic phenotypes. The dramatic outcome of Piezo blockade in wounds suggests that adipocyte-derived fibroblasts are important contributors to wound fibrosis and may have an outsized impact on scarring relative to their numbers in wounds (as a relative minority of wound fibroblasts). Our experiments into the effects of Piezo inhibition during the remodeling phase of healing have particularly exciting translational implications, as they indicate that Piezo inhibition could even rescue existing scars, which would have important therapeutic implications for the millions of patients who suffer from existing scars.

Results in our novel foreskin xenograft model are promising for ability to translate these findings to wound healing mediated by human cells. This model allowed us to examine the global effect of P1i/P2i on healing by human fibroblasts.

While the roles of canonical mechanotransduction (e.g., FAK, YAP) in driving pro-fibrotic fibroblast activity have been broadly studied, the present findings suggest an entirely new pathway and mechanism by which adipocyte mechanosensing can influence fibrosis. We have found that Piezo proteins are highly expressed on adipocytes and not fibroblasts; as contributions of adipocytes to scarring remain relatively unknown, and contributions of mechanosignaling to adipocyte biology even more so, these findings could open a new direction of inquiry through which a distinct cell lineage could be a functionally important and targetable driver of fibrosis. Our findings also raise the interesting possibility that distinct mechanical signaling pathways may be involved in driving fibrosis from the perspective of different cell types (e.g., adipocytes versus fibroblasts, or even different fibroblast subsets).

### F. REFERENCES

Aarabi, S., Bhatt, K. A., Shi, Y., Paterno, J., Chang, E. I., Loh, S. A., Holmes, J. W., Longaker, M. T., Yee, H., and Gurtner, G. C. (2007). Mechanical load initiates hypertrophic scar formation through decreased cellular apoptosis. FASEB J 21, 3250-3261. 10.1096/fj.07-8218com.
Almadori, A., Griffin, M., Ryan, C. M., Hunt, D. F., Hansen, E., Kumar, R., Abraham, D. J., Denton, C. P., and Butler, P. E. M. (2019). Stem cell enriched lipotransfer reverses the effects of fibrosis in systemic sclerosis. PLOS One 14, e0218068. 10.1371/journal.pone.0218068.
Bergen, V., Lange, M., Peidli, S., Wolf, F. A., and Theis, F. J. (2020a). Generalizing RNA velocity to transient cell states through dynamical modeling. Nature biotechnology. 10.1038/s41587-020-0591-3.
Bergen, V., Lange, M., Peidli, S., Wolf, F. A., and Theis, F. J. (2020b). Generalizing RNA velocity to transient cell states through dynamical modeling. Nat Biotechnol 38, 1408-1414. 10.1038/s41587-020-0591-3.
Chen, E. Y., Tan, C. M., Kou, Y., Duan, Q., Wang, Z., Meirelles, G. V., Clark, N. R., and Ma'ayan, A. (2013). Enrichr: interactive and collaborative HTML5 gene list enrichment analysis tool. BMC Bioinformatics 14, 128. 10.1186/1471-2105-14-128.
Chen, K., Henn, D., Sivaraj, D., Bonham, C. A., Griffin, M., Choi Kussie, H., Padmanabhan, J., Trotsyuk, A. A., Wan, D. C., Januszyk, M., et al. (2021a). Mechanical Strain Drives Myeloid Cell Differentiation Toward Pro-Inflammatory Subpopulations. Adv Wound Care (New Rochelle). 10.1089/wound.2021.0036.
Chen, K., Kwon, S. H., Henn, D., Kuehlmann, B. A., Tevlin, R., Bonham, C. A., Griffin, M., Trotsyuk, A. A., Borrelli, M. R., Noishiki, C., et al. (2021b). Disrupting biological sensors of force promotes tissue regeneration in large organisms. Nat Commun 12, 5256. 10.1038/s41467-021-25410-z.
Chen, K., Vigliotti, A., Bacca, M., McMeeking, R. M., Deshpande, V. S., and Holmes, J. W. (2018). Role of boundary conditions in determining cell alignment in response to stretch. Proc Natl Acad Sci USA 115, 986-991. 10.1073/pnas. 1715059115.
desJardins-Park, H. E., Gurtner, G. C., Wan, D. C., and Longaker, M. T. (2021). From Chronic Wounds to Scarring: The Growing Healthcare Burden of Under- and Over-healing Wounds. Adv Wound Care (New Rochelle). 10.1089/wound.2021.0039.
Dickson, I. (2020). Creeping fat in Crohn's disease explained. Nat Rev Gastroenterol Hepatol 17, 713. 10.1038/s41575-020-00379-0.
Driskell, R. R., Lichtenberger, B. M., Hoste, E., Kretzschmar, K., Simons, B. D., Charalambous, M., Ferron, S. R., Herault, Y., Pavlovic, G., Ferguson-Smith, A. C., and Watt, F. M. (2013). Distinct fibroblast lineages determine dermal architecture in skin development and repair. Nature 504, 277-281. 10.1038/nature12783.
Foster, D. S., Januszyk, M., Yost, K. E., Chinta, M. S., Gulati, G. S., Nguyen, A. T., Burcham, A. R., Salhotra, A., Ransom, R. C., Henn, D., et al. (2021). Integrated spatial multiomics reveals fibroblast fate during tissue repair. Proc Natl Acad Sci USA 118. 10.1073/pnas.2110025118.
Gerstner, N., Kehl, T., Lenhof, K., Müller, A., Mayer, C., Eckhart, L., Grammes, N. L., Diener, C., Hart, M., Hahn, O., et al. (2020). GeneTrail 3: advanced high-throughput enrichment analysis. Nucleic Acids Res 48, W515-W520. 10.1093/nar/gkaa306.
Goralski, K. B., Mccarthy, T. C., Hanniman, E. A., Zabel, B. A., Butcher, E. C., Parlee, S. D., Muruganandan, S., and Sinal, C. J. (2007). Chemerin, a novel adipokine that regulates adipogenesis and adipocyte metabolism. J Biol Chem 282, 28175-28188. 10.1074/jbc.M700793200.
Griffin, M. F., Borrelli, M. R., Garcia, J. T., Januszyk, M., King, M., Lerbs, T., Cui, L., Moore, A. L., Shen, A. H., Mascharak, S., et al. (2021). JUN promotes hypertrophic skin scarring via CD36 in preclinical in vitro and in vivo models. Sci Transl Med 13, eabb3312. 10.1126/scitranslmed.abb3312.
Griffin, M. F., desJardins-Park, H. E., Mascharak, S., Borrelli, M. R., and Longaker, M. T. (2020). Understanding the impact of fibroblast heterogeneity on skin fibrosis. Dis Model Mech 13. 10.1242/dmm.044164.
Guasti, L., Prasongchean, W., Kleftouris, G., Mukherjee, S., Thrasher, A. J., Bulstrode, N. W., and Ferretti, P. (2012). High plasticity of pediatric adipose tissue-derived stem cells: too much for selective skeletogenic differentiation? Stem Cells Transl Med 1, 384-395. 10.5966/sctm.2012-0009.
Gulati, G. S., Sikandar, S. S., Wesche, D. J., Manjunath, A., Bharadwaj, A., Berger, M. J., Ilagan, F., Kuo, A. H., Hsieh, R. W., Cai, S., et al. (2020). Single-cell transcriptional diversity is a hallmark of developmental potential. Science 367, 405-411. 10.1126/science.aax0249.
Gurtner, G. C., Werner, S., Barrandon, Y., and Longaker, M. T. (2008). Wound repair and regeneration. Nature 453, 314-321. 10.1038/nature07039.
Harris, R. B. (2014). Direct and indirect effects of leptin on adipocyte metabolism. Biochim Biophys Acta 1842, 414-423. 10.1016/j.bbadis.2013.05.009.
He, L., Si, G., Huang, J., Samuel, A. D. T., and Perrimon, N. (2018). Mechanical regulation of stem-cell differentiation by the stretch-activated Piezo channel. Nature 555, 103-106. 10.1038/nature25744.
Holt, J. R., Zeng, W. Z., Evans, E. L., Woo, S. H., Ma, S., Abuwarda, H., Loud, M., Patapoutian, A., and Pathak, M. M. (2021). Spatiotemporal dynamics of PIEZO1 localization controls keratinocyte migration during wound healing. Elife 10. 10.7554/eLife.65415.
Hossain, M. G., Iwata, T., Mizusawa, N., Shima, S. W., Okutsu, T., Ishimoto, K., and Yoshimoto, K. (2010). Compressive force inhibits adipogenesis through COX-2-mediated down-regulation of PPARgamma2 and C/EBPalpha. J Biosci Bioeng 109, 297-303. 10.1016/j.jbiosc.2009.09.003.
Ingram, J. R., Dougan, M., Rashidian, M., Knoll, M., Keliher, E. J., Garrett, S., Garforth, S., Blomberg, O. S., Espinosa, C., Bhan, A., et al. (2017). PD-L1 is an activation-independent marker of brown adipocytes. Nat Commun 8, 647. 10.1038/s41467-017-00799-8.
Jin, P., Jan, L. Y., and Jan, Y. N. (2020). Mechanosensitive Ion Channels: Structural Features Relevant to Mechanotransduction Mechanisms. Annu Rev Neurosci 43, 207-229. 10.1146/annurev-neuro-070918-050509.
Jin, S., Guerrero-Juarez, C. F., Zhang, L., Chang, I., Ramos, R., Kuan, C. H., Myung, P., Plikus, M. V., and Nie, Q. (2021). Inference and analysis of cell-cell communication using CellChat. Nat Commun 12, 1088. 10.1038/s41467-021-21246-9.
Kuehlmann, B., Bonham, C. A., Zucal, I., Prantl, L., and Gurtner, G. C. (2020). Mechanotransduction in Wound Healing and Fibrosis. J Clin Med 9. 10.3390/jcm9051423.
Leavitt, T., Hu, M. S., and Longaker, M. T. (2017). Isolation of Live Fibroblasts by Fluorescence-Activated Cell Sorting. Methods Mol Biol 1627, 205-212. 10.1007/978-1-4939-7113-8_13.
Marangoni, R. G., and Lu, T. T. (2017). The roles of dermal white adipose tissue loss in scleroderma skin fibrosis. Curr Opin Rheumatol 29, 585-590. 10.1097/BOR.0000000000000437.
Mascharak, S., desJardins-Park, H. E., Davitt, M. F., Griffin, M., Borrelli, M. R., Moore, A. L., Chen, K., Duoto, B., Chinta, M., Foster, D. S., et al. (2021a). Preventing Engrailed-1 activation in fibroblasts yields wound regeneration without scarring. Science 372. 10.1126/science.aba2374.
Mascharak, S., desJardins-Park, H. E., Davitt, M. F., Guardino, N. J., Gurtner, G. C., Wan, D. C., and Longaker, M. T. (2021b). Modulating Cellular Responses to Mechanical Forces to Promote Wound Regeneration. Adv Wound Care (New Rochelle). 10.1089/wound.2021.0040.
Mascharak, S., desJardins-Park, H. E., Januszyk, M., Chen, K., Davitt, M. F., Demeter, J., Henn, D., Griffin, M., Bonham, C. A., Foster, D. S., et al. (2020). Divergent molecular signatures of regeneration and fibrosis during wound repair. bioRxiv, 2020.2012.2017.423181. 10.1101/2020.12.17.423181.
Matsunaga, M., Kimura, M., Ouchi, T., Nakamura, T., Ohyama, S., Ando, M., Nomura, S., Azuma, T., Ichinohe, T., and Shibukawa, Y. (2021). Mechanical Stimulation-Induced Calcium Signaling by Piezo1 Channel Activation in Human Odontoblast Reduces Dentin Mineralization. Front Physiol 12, 704518. 10.3389/fphys.2021.704518.
Pethö, Z., Najder, K., Bulk, E., and Schwab, A. (2019). Mechanosensitive ion channels push cancer progression. Cell Calcium 80, 79-90. 10.1016/j.ceca.2019.03.007.
Plikus, M. V., Guerrero-Juarez, C. F., Ito, M., Li, Y. R., Dedhia, P. H., Zheng, Y., Shao, M., Gay, D. L., Ramos, R., Hsi, T. C., et al. (2017). Regeneration of fat cells from myofibroblasts during wound healing. Science 355, 748-752. 10.1126/science.aai8792.
Poglio, S., Galvani, S., Bour, S., André, M., Prunet-Marcassus, B., Pénicaud, L., Casteilla, L., and Cousin, B. (2009). Adipose tissue sensitivity to radiation exposure. Am J Pathol 174, 44-53. 10.2353/ajpath.2009.080505.
Romac, J. M., Shahid, R. A., Swain, S. M., Vigna, S. R., and Liddle, R. A. (2018). Piezo1 is a mechanically activated ion channel and mediates pressure induced pancreatitis. Nat Commun 9, 1715. 10.1038/s41467-018-04194-9.
Ruifrok, A. C., and Johnston, D. A. (2001). Quantification of histochemical staining by color deconvolution. Anal Quant Cytol Histol 23, 291-299.
Sakers, A., De Siqueira, M. K., Seale, P., and Villanueva, C. J. (2022). Adipose-tissue plasticity in health and disease. Cell 185, 419-446. 10.1016/j.cell.2021.12.016.
Shook, B. A., Wasko, R. R., Mano, O., Rutenberg-Schoenberg, M., Rudolph, M. C., Zirak, B., Rivera-Gonzalez, G. C., López-Giráldez, F., Zarini, S., Rezza, A., et al. (2020). Dermal Adipocyte Lipolysis and Myofibroblast Conversion Are Required for Efficient Skin Repair. Cell Stem Cell 26, 880-895.e886. 10.1016/j.stem.2020.03.013.
Strong, A. L., Rubin, J. P., Kozlow, J. H., and Cederna, P. S. (2019). Fat Grafting for the Treatment of Scleroderma. Plast Reconstr Surg 144, 1498-1507. 10.1097/PRS.0000000000006291.
Stuart, T., Butler, A., Hoffman, P., Hafemeister, C., Papalexi, E., Mauck, W. M., 3rd, Hao, Y., Stoeckius, M., Smibert, P., and Satija, R. (2019). Comprehensive Integration of Single-Cell Data. Cell 177, 1888-1902 e1821. 10.1016/j.cell.2019.05.031.
Syeda, R., Xu, J., Dubin, A. E., Coste, B., Mathur, J., Huynh, T., Matzen, J., Lao, J., Tully, D. C., Engels, I. H., et al. (2015). Chemical activation of the mechanotransduction channel Piezo1. Elife 4. 10.7554/eLife.07369.
Trapnell, C., Cacchiarelli, D., Grimsby, J., Pokharel, P., Li, S., Morse, M., Lennon, N. J., Livak, K. J., Mikkelsen, T. S., and Rinn, J. L. (2014). The dynamics and regulators of cell fate decisions are revealed by pseudotemporal ordering of single cells. Nature biotechnology 32, 381-386. 10.1038/nbt.2859.
Vishvanath, L., and Gupta, R. K. (2019). Contribution of adipogenesis to healthy adipose tissue expansion in obesity. J Clin Invest 129, 4022-4031. 10.1172/JCI129191.
Wolf, F. A., Angerer, P., and Theis, F. J. (2018). SCANPY: large-scale single-cell gene expression data analysis. Genome Biol 19, 15. 10.1186/s13059-017-1382-0.
Wong, V. W., Bhatt, K. A., Vial, I. N., Wu, K., Padois, K., Dauskardt, R. H., Longaker, M. T., and Gurtner, G. C. (2010). Beyond Langer's lines: Manipulating wound mechanical forces to control hypertrophic scar formation in the red Duroc pig. Journal of the American College of Surgeons 211, S77. 10.1016/j.jamcollsurg.2010.06.202.
Yuan, Y., Gao, J., and Ogawa, R. (2015). Mechanobiology and Mechanotherapy of Adipose Tissue-Effect of Mechanical Force on Fat Tissue Engineering. Plast Reconstr Surg Glob Open 3, e578. 10.1097/GOX.0000000000000564.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

1. A method of promoting healing of a wound in a dermal location of a subject, the method comprising:
administering an effective amount of a Piezo inhibitor to the wound to promote healing of the wound.

2. The method of claim 1, wherein the method comprises reducing a transition of adipocytes to fibroblasts (EPFs) in the wound.

3. The method of claim 1, wherein the method comprises preserving an amount of adipocytes relative to an amount of fibroblasts present in the wound.

4. The method of claim 1, wherein the administering comprises injecting the composition below a topical dermal location of the subject.

5. The method of claim 1, wherein the Piezo inhibitor comprises an inhibitor selected from the group consisting of a Piezo 1 inhibitor, a Piezo 2 inhibitor and combinations thereof.

6. The method of claim 5, wherein the Piezo inhibitor comprises a Piezo 1 inhibitor.

7. The method of claim 5, wherein the Piezo inhibitor comprises a Piezo 2 inhibitor.

8. The method of claim 1, wherein the method further comprises administering a YAP inhibitor.

9. The method of claim 8, wherein the YAP inhibitor is verteporfin.

10. The method of claim 1, wherein the subject is an adult.

11. The method of claim 1, wherein the comprises regeneration of dermal appendages.

12. The method of claim 11, wherein the dermal appendages comprise hair follicles, sweat glands, and sebaceous glands.

13. The method of claim 1, wherein the method further comprises forming the wound.

14. The method of claim 1, wherein the wound is a surgical wound.

15. The method of claim 1, wherein the method produces a healed wound with reduced levels of scarring compared to a control.

16. The method of claim 1, wherein the method produces a scarless healed wound.

17-29. (canceled)

30. A method of promoting hair growth on a subject, the method comprising:
forming a wound in a dermal location of a subject, and
administering an effective amount of a Piezo inhibitor composition to the wound to promote hair growth on the subject.

31. The method of claim 30, wherein the hair growth comprises generating a new hair follicle.

32. The method of claim 30, wherein the dermal location is hairless.

33. (canceled)

34. The method of claim 30, wherein the dermal location is present on the scalp of the subject.

35-50. (canceled)
